SEQUENCE LISTING (43 sequences)

SEQ ID NO: 1 - protein, 1045 aa, synthetic construct
MKKVEVSRPYQSLLLPNHRKFKYLDETWNAYKSVKSLLHRFLVCAYGAVPFNKFVEVVEKVDNDQLVLAFAVRLFRLVPVESTSFAKVDKANLAKSLANHLPVGTAIPANVQSYFDSNFDPKKYMWIDCAWEADRLAREMGLSASQFSEYATTMLWEDWLPLNKDDVNGWGSVSGLFGEGKKEDRQQKVKMLNNLLNGIKKNPPKDYTQYLKILLNAFDAKSHKEAVKNYKGDSTGRTASYLSEKSGEITELMLEQLMSNIQRDIGDKQKEISLPKKDVVKKYLESESGVPYDQNLWSQAYRNAASSIKKTDTRNFNSTLEKFKNEVELRGLLSEGDDVEILRSKFFSSEFHKTPDKFVIKPEHIGFNNKYNVVAELYKLKAEATDFESAFATVKDEFEEKGIKHPIKNILEYIWNNEVPVEKWGRVARFNQSEEKLLRIKANPTVECNQGMTFGNSAMVGEVLRSNYVSKKGALVSGEHGGRLIGQNNMIWLEMRLLNKGKWETHHVPTHNMKFFEEVHAYNPSLADSVNVRNRLYRSEDYTQLPSSITDGLKGNPKAKLLKRQHCALNNMTANVLNPKLSFTINKKNDDYTVIIVHSVEVSKPRREVLVGDYLVGMDQNQTASNTYAVMQVVKPKSTDAIPFRNMWVRFVESGSIESRTLNSRGEYVDQLNHDGVDLFEIGDTEWVDSARKFFNKLGVKHKDGTLVDLSTAPRKAYAFNNFYFKTMLNHLRSNEVDLTLLRNEILRVANGRFSPMRLGSLSWTTLKALGSFKSLVLSYFDRLGAKEMVDKEAKDKSLFDLLVAINNKRSNKREERTSRIASSLMTVAQKYKVDNAVVHVVVEGNLSSTDRSASKAHNRNTMDWCSRAVVKKLEDMCNLYGFNIKGVPAFYTSHQDPLVHRADYDDPKPALRCRYSSYSRADFSKWGQNALAAVVRWASNKKSNTCYKVGAVEFLKQHGLFADKKLTVEQFLSKVKDEEILIPRRGGRVFLTTHRLLAESTFVYLNGVKYHSCNADEVAAVNICLNDWVIPCKKKMKEESSASG

SEQ ID NO: 2 - DNA, 790 bp, synthetic construct
  source 1..790: mol_type = other DNA
tctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgc

SEQ ID NO: 3 - DNA, 8131 bp, synthetic construct
  source 1..8131: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgtcgcgaggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaagcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 4 - DNA, 28 bp, synthetic construct
  source 1..28: mol_type = other DNA
cgtttaaacttaagcttaccggtgccac

SEQ ID NO: 5 - DNA, 24 bp, synthetic construct
  source 1..24: mol_type = other DNA
tgtgcatagtcacaccgtcgcgag

SEQ ID NO: 6 - DNA, 36 bp, synthetic construct
  source 1..36: mol_type = other DNA
ctgatgagcagaattcagagagacatcggcgacaag

SEQ ID NO: 7 - DNA, 33 bp, synthetic construct
  source 1..33: mol_type = other DNA
ctctgaattctgctcatcagctgctccagcatc

SEQ ID NO: 8 - DNA, 9099 bp, synthetic construct
  source 1..9099: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacgtcgacatgaagaaggtggaggtgagcagaccctatcagagcctgctcctgcccaaccacagaaagttcaagtacctggacgaaacctggaacgcctacaagagcgtgaagtccctgctgcacagattcctggtgtgcgcctacggcgccgtgcccttcaacaagttcgtggaggtggtggagaaagtggacaacgaccaactggtgctggccttcgccgtgagactgttcagactggtccccgtggagagcacatccttcgccaaggtggacaaagctaacctggccaagagcctggccaaccacctgcccgtgggcaccgccatccccgccaacgtgcagagctatttcgacagcaacttcgaccccaagaagtacatgtggatcgactgcgcctgggaggccgacagactggctagagagatgggcctgagcgctagccaattcagcgagtacgccaccaccatgctgtgggaggactggctgcccctgaacaaggacgacgtgaacggctggggcagcgtgagcggcctgttcggcgagggcaagaaggaggacagacagcagaaggtgaagatgctgaacaacctgctgaacggcattaagaagaatccccccaaggactatacacagtacctgaagatcctgctgaacgccttcgacgccaagagccacaaggaggccgtgaagaactacaagggcgacagcaccggcagaaccgctagctacctgagcgagaagagcggcgagatcaccgagctgatgctggagcagctgatgagcaacattcagagagacatcggcgacaagcagaaggagatcagcctgcccaagaaggacgtggtcaaaaagtacctggagagcgagagcggcgtgccctacgatcagaacctgtggagccaagcctacagaaacgccgctagctccatcaaaaagaccgacacaagaaacttcaacagcaccctggagaagttcaagaacgaggtggagctgagaggcctgctgagcgagggcgacgacgtggagatcctgagaagcaagttcttcagcagcgagttccacaagacccccgacaagttcgtgatcaagcccgagcacatcggcttcaacaacaagtacaacgtggtggccgagctgtacaagctgaaggccgaggccaccgacttcgagagcgccttcgccaccgtcaaggacgaatttgaggagaagggcatcaagcaccccatcaagaacatcctggagtacatctggaacaacgaggtgcccgtggagaagtggggcagagtggctagattcaatcagagcgaggagaagctgctgagaatcaaggccaaccccaccgtggagtgcaaccaaggcatgaccttcggcaacagcgccatggtgggcgaggtgctgagaagcaactacgtgagcaagaagggcgccctggtgagcggcgagcacggcggcagactgatcgggcagaacaacatgatctggctggagatgagactgctgaacaagggcaagtgggaaacccaccacgtgcccacccacaacatgaagttcttcgaagaggtgcacgcctacaaccctagcctggccgacagcgtgaacgtgagaaacagactgtacagaagcgaggactacacacagctgcctagcagcattaccgacggcctgaagggcaaccccaaggccaagctgctgaagagacagcactgcgccctgaacaacatgaccgccaatgtgctcaaccccaagctgagcttcaccatcaacaagaagaacgacgactacaccgtgatcatcgtgcacagcgtcgaggtgagcaagcctagaagagaggtgctggtgggcgactacctggtgggcatggatcagaatcagaccgctagcaacacctacgccgtgatgcaagtggtgaagcccaagagcaccgacgccatccccttcagaaacatgtgggtgagattcgtggagagcggcagcatcgagagcagaaccctgaacagcagaggcgagtacgtggatcagctgaaccacgacggcgtggacctgttcgagatcggcgacaccgagtgggtggacagcgctagaaagttcttcaacaagctgggcgtgaagcacaaggacggcaccctggtggacctgagcaccgcccctagaaaggcctacgccttcaacaacttctacttcaagaccatgctgaaccacctccggagcaacgaggtggacctgaccctgctgagaaacgagatcctcagagtggctaacggcagattcagccccatgagactgggcagcctgagctggaccaccctgaaggccctgggcagcttcaagagcctggtgctgagctacttcgacagactgggcgccaaggagatggtggacaaggaggccaaggacaagagcctgttcgacctgctggtggccatcaacaacaagagaagcaacaagagagaggagagaacaagcagaatcgctagcagcctgatgaccgtggctcagaagtacaaagtggacaacgccgtggtgcacgtggtcgtggagggcaacctgagcagcaccgacagaagcgctagcaaggcccacaacagaaacaccatggactggtgcagcagagccgtggtgaagaaactggaggacatgtgcaacctgtacggcttcaacatcaagggcgtgcccgccttctacacaagccaccaagaccccctggtgcacagagccgactacgacgaccccaagcccgccctgagatgcagatacagcagctacagcagagccgacttcagcaagtgggggcagaacgccctggccgccgtggtgagatgggctagcaacaagaagagcaacacctgctacaaggtgggcgccgtggagttcctgaagcagcacggcctgttcgccgacaagaagctgaccgtggagcagttcctgagcaaggtgaaggacgaggagatcctgatccctagaagaggcggcagagtgttcctgaccacccacagactgctggccgagagcaccttcgtgtacctgaacggcgtgaagtaccacagctgcaacgccgacgaggtggccgccgtgaacatctgcctgaacgactgggtgatcccctgcaagaaaaagatgaaggaggagagcagcgctagcggcacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaacccttgctcacctcgcgacggtgtgactatgcacacattctctcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 9099 bp, synthetic construct
  source 1..9099: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacgtcgacatgaagaaggtggaggtgagcagaccctatcagagcctgctcctgcccaaccacagaaagttcaagtacctggacgaaacctggaacgcctacaagagcgtgaagtccctgctgcacagattcctggtgtgcgcctacggcgccgtgcccttcaacaagttcgtggaggtggtggagaaagtggacaacgaccaactggtgctggccttcgccgtgagactgttcagactggtccccgtggagagcacatccttcgccaaggtggacaaagctaacctggccaagagcctggccaaccacctgcccgtgggcaccgccatccccgccaacgtgcagagctatttcgacagcaacttcgaccccaagaagtacatgtggatcgactgcgcctgggaggccgacagactggctagagagatgggcctgagcgctagccaattcagcgagtacgccaccaccatgctgtgggaggactggctgcccctgaacaaggacgacgtgaacggctggggcagcgtgagcggcctgttcggcgagggcaagaaggaggacagacagcagaaggtgaagatgctgaacaacctgctgaacggcattaagaagaatccccccaaggactatacacagtacctgaagatcctgctgaacgccttcgacgccaagagccacaaggaggccgtgaagaactacaagggcgacagcaccggcagaaccgctagctacctgagcgagaagagcggcgagatcaccgagctgatgctggagcagctgatgagcagaattcagagagacatcggcgacaagcagaaggagatcagcctgcccaagaaggacgtggtcaaaaagtacctggagagcgagagcggcgtgccctacgatcagaacctgtggagccaagcctacagaaacgccgctagctccatcaaaaagaccgacacaagaaacttcaacagcaccctggagaagttcaagaacgaggtggagctgagaggcctgctgagcgagggcgacgacgtggagatcctgagaagcaagttcttcagcagcgagttccacaagacccccgacaagttcgtgatcaagcccgagcacatcggcttcaacaacaagtacaacgtggtggccgagctgtacaagctgaaggccgaggccaccgacttcgagagcgccttcgccaccgtcaaggacgaatttgaggagaagggcatcaagcaccccatcaagaacatcctggagtacatctggaacaacgaggtgcccgtggagaagtggggcagagtggctagattcaatcagagcgaggagaagctgctgagaatcaaggccaaccccaccgtggagtgcaaccaaggcatgaccttcggcaacagcgccatggtgggcgaggtgctgagaagcaactacgtgagcaagaagggcgccctggtgagcggcgagcacggcggcagactgatcgggcagaacaacatgatctggctggagatgagactgctgaacaagggcaagtgggaaacccaccacgtgcccacccacaacatgaagttcttcgaagaggtgcacgcctacaaccctagcctggccgacagcgtgaacgtgagaaacagactgtacagaagcgaggactacacacagctgcctagcagcattaccgacggcctgaagggcaaccccaaggccaagctgctgaagagacagcactgcgccctgaacaacatgaccgccaatgtgctcaaccccaagctgagcttcaccatcaacaagaagaacgacgactacaccgtgatcatcgtgcacagcgtcgaggtgagcaagcctagaagagaggtgctggtgggcgactacctggtgggcatggatcagaatcagaccgctagcaacacctacgccgtgatgcaagtggtgaagcccaagagcaccgacgccatccccttcagaaacatgtgggtgagattcgtggagagcggcagcatcgagagcagaaccctgaacagcagaggcgagtacgtggatcagctgaaccacgacggcgtggacctgttcgagatcggcgacaccgagtgggtggacagcgctagaaagttcttcaacaagctgggcgtgaagcacaaggacggcaccctggtggacctgagcaccgcccctagaaaggcctacgccttcaacaacttctacttcaagaccatgctgaaccacctccggagcaacgaggtggacctgaccctgctgagaaacgagatcctcagagtggctaacggcagattcagccccatgagactgggcagcctgagctggaccaccctgaaggccctgggcagcttcaagagcctggtgctgagctacttcgacagactgggcgccaaggagatggtggacaaggaggccaaggacaagagcctgttcgacctgctggtggccatcaacaacaagagaagcaacaagagagaggagagaacaagcagaatcgctagcagcctgatgaccgtggctcagaagtacaaagtggacaacgccgtggtgcacgtggtcgtggagggcaacctgagcagcaccgacagaagcgctagcaaggcccacaacagaaacaccatggactggtgcagcagagccgtggtgaagaaactggaggacatgtgcaacctgtacggcttcaacatcaagggcgtgcccgccttctacacaagccaccaagaccccctggtgcacagagccgactacgacgaccccaagcccgccctgagatgcagatacagcagctacagcagagccgacttcagcaagtgggggcagaacgccctggccgccgtggtgagatgggctagcaacaagaagagcaacacctgctacaaggtgggcgccgtggagttcctgaagcagcacggcctgttcgccgacaagaagctgaccgtggagcagttcctgagcaaggtgaaggacgaggagatcctgatccctagaagaggcggcagagtgttcctgaccacccacagactgctggccgagagcaccttcgtgtacctgaacggcgtgaagtaccacagctgcaacgccgacgaggtggccgccgtgaacatctgcctgaacgactgggtgatcccctgcaagaaaaagatgaaggaggagagcagcgctagcggcacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaacccttgctcacctcgcgacggtgtgactatgcacacattctctcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 10 - RNA, 43 nt, synthetic construct
  source 1..43: mol_type = other RNA
agagaatgtgtgcatagtcacaccagtaagatttggtgtctat

SEQ ID NO: 11 - RNA, 47 nt, synthetic construct
  source 1..47: mol_type = other RNA
agagaatgtgtgcatagtcacactatgcagaaatattgctattgcct

SEQ ID NO: 12 - RNA, 43 nt, synthetic construct
  source 1..43: mol_type = other RNA
agagaatgtgtgcatagtcacacacaaggcaaacttgaccaat

SEQ ID NO: 13 - DNA, 2672 bp, synthetic construct
  source 1..2672: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagagaatgtgtgcatagtcacacacaaggcaaacttgaccaatttttttctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 14 - RNA, 20 nt, synthetic construct
  source 1..20: mol_type = other RNA
cagtaagatttggtgtctat

SEQ ID NO: 15 - RNA, 24 nt, synthetic construct
  source 1..24: mol_type = other RNA
tatgcagaaatattgctattgcct

SEQ ID NO: 16 - RNA, 20 nt, synthetic construct
  source 1..20: mol_type = other RNA
acaaggcaaacttgaccaat

SEQ ID NO: 17 - RNA, 23 nt, synthetic construct
  source 1..23: mol_type = other RNA
agagaatgtgtgcatagtcacac

SEQ ID NO: 18 - protein, 1112 aa, synthetic construct
MEAGKVGKKGKTNKKFIIRPYLTELNLREDGRLAFQKTFDYMDEQQAALFILGGSVMSHLDESIIRRLGLHKGSKKDLPQRLRVSLHLAIARFRLVSVNYHLDAKAISRMSPTARLAHKQLAEAHRASLIKSPISVWRNRHGVPDEAVHAYLDGNYDPETYAWQDTAMLAKKLCGILKLSPEDFKEASEAMMRNVNFLGCSGSTGSGSSVSNLFGQNEKEDSRNQARIESKTAKVIGKLLESRKPIPMERAVSLVCKSLGHPDAEAAGEDHGGQTDKSTFRQFMRGEYGGSLKELAKKLQKDAHKHRNKSIIPHRETIGAFIKQCASGEFYNKATSESWKDFNAMMNGKYKHNIIFVTEKIALGNAMRKLESNEEAVKASKQLEKLIDKYEMDGNKRFVPKVASVNGLEAYRDVIQDNPQEDDESVKDWLKRLWITFSEGNDRRLRTPFRWLVESLARLETPESAIKDGCRLMSIRHKHESQRPHPFVRVQSRFTVGDSNIAGSINKPTELKPNRDGRSPEDYWGGNPVVWMSCRLLNGNRWQDMRIPIHNSRYINEVYYTRMGPDGNHALPLKEHARDVKHDYEAETKISRTAARRVNENRMLRRGKPSKRFERVKANSTHNVVFDPKTTASFNRRQDDIYGTINHRHPMVPLAPDGVFAVGANIIGIDLGESVPLAAGILQKCTSTDSEAVRYACGHWKVVGMGKPGQLLDRQTSAIRRKQPHTIIDPMSNMGEPFSSPICQKFIGKCRKFVRAKGSEEDNKAFDDMVKREPSLYSFHGRWGWLLKQMMKAAKGARLDPFREHLEWLLFRTKYGPTNRKSLNLNSMASTKNVISAIDSYMSRRGWKTVEQRQRRDGRLQAARSSLQSNLVNRRLERIKKEESQCIRLAHLFAVTTHLVLEDNLPKQNGASSRADNGRKADWRSRHLAQRLCGIKNDSGSCAIAGVRARRIDPVMTSHMDPFVYSLDNKWAMRARYTKVSLSSMTDYHANLIRSILLEKPGKRQTTEYYQRAVQDFARLHGLDVDEMRKVREACWYKKRIKAKTLYIPCRGGRVFLSTHRLQSDTPHTDSSGAVLWEQDADQVAALNVALRYIDQCYRSNKKGLAKVKKPK

SEQ ID NO: 19 - DNA, 9310 bp, synthetic construct
  source 1..9310: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccggtgccaccatgccggcagctaagaaaaagaaactggatggcagcgtcgacatggaggccggcaaggtgggcaagaagggcaagaccaacaagaagttcatcatcagaccctacctgaccgagctgaacctgagagaggacgggagactggcttttcaaaagaccttcgactacatggacgagcagcaagccgccctgttcatcctgggcggcagcgtgatgagccacctggacgagagcatcatccggagactgggcctgcacaagggcagcaagaaggacctgcctcagagactgagagtgagcctgcacctggccatcgctagattcagactggtgagcgtgaactaccacctggacgccaaggccatcagcagaatgagccccaccgctagactggcccacaagcagctggccgaggcccacagagctagcctgatcaagagccccatcagcgtgtggagaaacagacacggcgtgcccgacgaggccgtgcacgcctacctggacggcaactacgaccccgaaacctacgcctggcaagacaccgccatgctggccaagaagctgtgcggcatcctgaagctgagccccgaggactttaaggaggctagcgaggccatgatgagaaacgtgaacttcctgggctgcagcggcagcaccggctccggcagcagcgtgagcaacctgttcgggcagaatgagaaggaggacagcagaaaccaagctagaatcgaaagcaagaccgctaaggtgatcggcaagctgctggagagcagaaagcccatccccatggagagagccgtgagcctggtgtgcaaatccctcggccaccccgacgctgaggccgccggggaggaccacggcgggcagaccgacaagagcaccttcagacagttcatgagaggcgagtacggcggcagcctgaaggagctggccaaaaagctccaaaaggacgcccacaagcacagaaacaagagcatcatcccccacagagaaaccatcggcgccttcatcaagcagtgcgctagcggcgagttctacaacaaggccacaagcgagagctggaaggacttcaacgccatgatgaacggcaagtacaagcacaacatcatcttcgtgaccgagaagatcgccctgggcaacgccatgagaaagctggagagcaacgaggaggccgtgaaggctagcaagcagctggagaagctgatcgacaagtacgagatggacggcaacaagagattcgtgcccaaggtggctagcgtgaacggcctggaggcctacagagacgtgatccaagacaacccccaagaggacgacgagagcgtgaaggactggctgaagagactgtggatcaccttcagcgagggcaacgaccggagactgagaacccccttcagatggctggtggagagcctggctagactggaaacccccgagagcgccatcaaggacggctgcagactgatgagcatcagacacaagcacgagagccaaagaccccaccccttcgtgagagtgcagagcagattcaccgtgggcgacagcaacatcgccggcagcatcaacaagcccaccgagctgaagcccaaccgggacggcagatcccccgaggactattggggcggcaaccccgtggtgtggatgagctgcagactgctgaacggcaacagatggcaagacatgagaatccccatccacaacagcagatacatcaacgaggtgtactacacaagaatgggccccgacggcaaccacgccctgcccctgaaggagcacgctagagacgtgaagcacgactacgaggccgaaaccaagatcagcagaaccgccgctagaagagtgaacgagaacagaatgctgagaagaggcaagcctagcaagagattcgagagagtgaaggccaacagcacccacaacgtggtgttcgaccccaagaccaccgctagcttcaaccggagacaagacgacatctacggcaccatcaaccacagacaccccatggtgcccctggcccctgatggcgtgttcgccgtgggcgccaacatcatcggcatcgacctgggcgagagcgtgcctctcgccgccggcatcctgcagaagtgcacaagcaccgacagcgaggccgtgagatacgcctgcggccactggaaggtggtgggcatgggcaagcccgggcagctgctggacagacagacaagcgccatcagaagaaagcagccccacaccatcatcgaccccatgagcaacatgggcgagcccttcagcagccccatctgtcagaagttcatcggcaagtgcagaaagttcgtgagagccaagggcagcgaggaggacaacaaggccttcgacgacatggtgaagagagagcctagcctgtacagcttccacggcagatggggctggctgctgaagcagatgatgaaggccgccaagggcgctagactggaccccttcagagagcacctggagtggctgctgttcagaaccaagtacggccccaccaacagaaagagcctgaacctgaacagcatggctagcaccaagaacgtgatcagcgccatcgacagctacatgagcagaagaggctggaaaaccgtggagcagagacagagaagagacggcagactgcaagccgctagaagcagcctgcagagcaacctggtgaaccggagactggagagaatcaagaaggaggagagccaatgcatcagactcgcccacctgttcgccgtgaccacccacctggtgctggaggacaacctgcccaagcagaacggcgctagcagcagagccgacaacggcagaaaggccgactggagaagcagacacctggctcagagactgtgtgggatcaagaacgacagcggcagctgcgccatcgccggcgtgagagctagaagaatcgaccccgtgatgacaagccacatggaccccttcgtgtacagcctggacaacaagtgggccatgagagctagatacaccaaggtgagcctgagcagcatgaccgactaccacgccaacctgatcagaagcatcctgctggagaagcccggcaagagacagaccaccgagtactatcagagagccgtgcaagacttcgctagactgcacggcctggacgtggacgagatgagaaaggtgagagaggcctgctggtacaagaagagaatcaaggccaagaccctgtacatcccctgcagaggcggcagagtgttcctgagcacccatcggctgcagagcgacacaccccacaccgacagcagcggcgccgtgctctgggaacaagacgccgaccaagtggccgccctgaacgtggccctgagatacatcgatcagtgctacagaagcaacaagaagggcctggccaaggtgaagaagcccaagacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgagaattccccttgagcatctgacttctggctaataaaggaaatttattttcattgcaatagtgtgttggaattttttgtgtctctcaggtaccaaaaacggctcggagatcatcattgcgtgcccaatcatcgtgggcattctaaatctagtcgaggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 20 - DNA, 8875 bp, synthetic construct
  source 1..8875: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccatatggctagcatgactggtggacagcaaatgggtcgcggatccatgccggcagctaagaaaaagaaactggatggcagcgtcgacatggaggccggcaaggtgggcaagaagggcaagaccaacaagaagttcatcatcagaccctacctgaccgagctgaacctgagagaggacgggagactggcttttcaaaagaccttcgactacatggacgagcagcaagccgccctgttcatcctgggcggcagcgtgatgagccacctggacgagagcatcatccggagactgggcctgcacaagggcagcaagaaggacctgcctcagagactgagagtgagcctgcacctggccatcgctagattcagactggtgagcgtgaactaccacctggacgccaaggccatcagcagaatgagccccaccgctagactggcccacaagcagctggccgaggcccacagagctagcctgatcaagagccccatcagcgtgtggagaaacagacacggcgtgcccgacgaggccgtgcacgcctacctggacggcaactacgaccccgaaacctacgcctggcaagacaccgccatgctggccaagaagctgtgcggcatcctgaagctgagccccgaggactttaaggaggctagcgaggccatgatgagaaacgtgaacttcctgggctgcagcggcagcaccggctccggcagcagcgtgagcaacctgttcgggcagaatgagaaggaggacagcagaaaccaagctagaatcgaaagcaagaccgctaaggtgatcggcaagctgctggagagcagaaagcccatccccatggagagagccgtgagcctggtgtgcaaatccctcggccaccccgacgctgaggccgccggggaggaccacggcgggcagaccgacaagagcaccttcagacagttcatgagaggcgagtacggcggcagcctgaaggagctggccaaaaagctccaaaaggacgcccacaagcacagaaacaagagcatcatcccccacagagaaaccatcggcgccttcatcaagcagtgcgctagcggcgagttctacaacaaggccacaagcgagagctggaaggacttcaacgccatgatgaacggcaagtacaagcacaacatcatcttcgtgaccgagaagatcgccctgggcaacgccatgagaaagctggagagcaacgaggaggccgtgaaggctagcaagcagctggagaagctgatcgacaagtacgagatggacggcaacaagagattcgtgcccaaggtggctagcgtgaacggcctggaggcctacagagacgtgatccaagacaacccccaagaggacgacgagagcgtgaaggactggctgaagagactgtggatcaccttcagcgagggcaacgaccggagactgagaacccccttcagatggctggtggagagcctggctagactggaaacccccgagagcgccatcaaggacggctgcagactgatgagcatcagacacaagcacgagagccaaagaccccaccccttcgtgagagtgcagagcagattcaccgtgggcgacagcaacatcgccggcagcatcaacaagcccaccgagctgaagcccaaccgggacggcagatcccccgaggactattggggcggcaaccccgtggtgtggatgagctgcagactgctgaacggcaacagatggcaagacatgagaatccccatccacaacagcagatacatcaacgaggtgtactacacaagaatgggccccgacggcaaccacgccctgcccctgaaggagcacgctagagacgtgaagcacgactacgaggccgaaaccaagatcagcagaaccgccgctagaagagtgaacgagaacagaatgctgagaagaggcaagcctagcaagagattcgagagagtgaaggccaacagcacccacaacgtggtgttcgaccccaagaccaccgctagcttcaaccggagacaagacgacatctacggcaccatcaaccacagacaccccatggtgcccctggcccctgatggcgtgttcgccgtgggcgccaacatcatcggcatcgacctgggcgagagcgtgcctctcgccgccggcatcctgcagaagtgcacaagcaccgacagcgaggccgtgagatacgcctgcggccactggaaggtggtgggcatgggcaagcccgggcagctgctggacagacagacaagcgccatcagaagaaagcagccccacaccatcatcgaccccatgagcaacatgggcgagcccttcagcagccccatctgtcagaagttcatcggcaagtgcagaaagttcgtgagagccaagggcagcgaggaggacaacaaggccttcgacgacatggtgaagagagagcctagcctgtacagcttccacggcagatggggctggctgctgaagcagatgatgaaggccgccaagggcgctagactggaccccttcagagagcacctggagtggctgctgttcagaaccaagtacggccccaccaacagaaagagcctgaacctgaacagcatggctagcaccaagaacgtgatcagcgccatcgacagctacatgagcagaagaggctggaaaaccgtggagcagagacagagaagagacggcagactgcaagccgctagaagcagcctgcagagcaacctggtgaaccggagactggagagaatcaagaaggaggagagccaatgcatcagactcgcccacctgttcgccgtgaccacccacctggtgctggaggacaacctgcccaagcagaacggcgctagcagcagagccgacaacggcagaaaggccgactggagaagcagacacctggctcagagactgtgtgggatcaagaacgacagcggcagctgcgccatcgccggcgtgagagctagaagaatcgaccccgtgatgacaagccacatggaccccttcgtgtacagcctggacaacaagtgggccatgagagctagatacaccaaggtgagcctgagcagcatgaccgactaccacgccaacctgatcagaagcatcctgctggagaagcccggcaagagacagaccaccgagtactatcagagagccgtgcaagacttcgctagactgcacggcctggacgtggacgagatgagaaaggtgagagaggcctgctggtacaagaagagaatcaaggccaagaccctgtacatcccctgcagaggcggcagagtgttcctgagcacccatcggctgcagagcgacacaccccacaccgacagcagcggcgccgtgctctgggaacaagacgccgaccaagtggccgccctgaacgtggccctgagatacatcgatcagtgctacagaagcaacaagaagggcctggccaaggtgaagaagcccaagacaggcggcggccccggcggcggcgccgccgccggcagcggcagccctaagaaaaaacgaaaagttggcagcggaagcaaaaggccggcggccacgaaaaaggccggccaggcaaaaaagaaaaagctcgagtacccatacgatgttccagattacgcttgactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 21 - DNA, 59 bp, synthetic construct
  source 1..59: mol_type = other DNA
acagcaaatgggtcgcggatccatgccggcagctaagaaaaagaaactggatggcagcg

SEQ ID NO: 22 - DNA, 55 bp, synthetic construct
  source 1..55: mol_type = other DNA
tctcagtggtggtggtggtggtgctcgagtcaagcgtaatctggaacatcgtatg

SEQ ID NO: 23 - DNA, 273 bp, synthetic construct
  source 1..273: mol_type = other DNA
ggagttcagacgtgtgctcttccgatctcagcacaaaaggaaactcaccctaactgtaaagtaattgtgtgttttgagactataaatatgcatgcgagaaaagccttgtttgccaccatggaacggctcggagatcatcattgcgnnnnnnngtgagcaagggcgaggagctgttcaccggggtggtgcccatcctggtcgagctggacggcgacgtaaacggccacaagttcagcgtgtccggcagatcggaagagcacacgtctgaactcc

SEQ ID NO: 24 - RNA, 61 nt, synthetic construct
  source 1..61: mol_type = other RNA
cctcgactagatttagaatgcccacgatgattgggcagtgagcaagggcgaggagctgttc

SEQ ID NO: 25 - RNA, 63 nt, synthetic construct
  source 1..63: mol_type = other RNA
cctcgactagatttagaatgcccacgatgattgggcacgcaatgatgatctccgagccgttcc

SEQ ID NO: 26 - RNA, 37 nt, synthetic construct
  source 1..37: mol_type = other RNA
cctcgactagatttagaatgcccacgatgattgggca

SEQ ID NO: 27 - RNA, 24 nt, synthetic construct
  source 1..24: mol_type = other RNA
gtgagcaagggcgaggagctgttc

SEQ ID NO: 28 - RNA, 26 nt, synthetic construct
  source 1..26: mol_type = other RNA
cgcaatgatgatctccgagccgttcc

SEQ ID NO: 29 - DNA, 59 bp, synthetic construct
  source 1..59: mol_type = other DNA
  misc_binding 1: bound_moiety = Biosg
gttgacatgctggattgagacttcctacactctttccctacacgacgctcttccgatct

SEQ ID NO: 30 - DNA, 58 bp, synthetic construct
  source 1..58: mol_type = other DNA
gatcggaagagcgtcgtgtagggaaagagtgtaggaagtctcaatccagcatgtcaac

SEQ ID NO: 31 - DNA, 19 bp, synthetic construct
  source 1..19: mol_type = other DNA
ggagttcagacgtgtgctc

SEQ ID NO: 32 - DNA, 24 bp, synthetic construct
  source 1..24: mol_type = other DNA
gttgacatgctggattgagacttc

SEQ ID NO: 33 - DNA, 58 bp, synthetic construct
  source 1..58: mol_type = other DNA
aatgatacggcgaccaccgagatctacactatagcctacactctttccctacacgacg

SEQ ID NO: 34 - DNA, 57 bp, synthetic construct
  source 1..57: mol_type = other DNA
caagcagaagacggcatacgagatcgagtaatgtgactggagttcagacgtgtgctc

SEQ ID NO: 35 - DNA, 1015 bp, synthetic construct
  source 1..1015: mol_type = other DNA
gggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagcgagaaaagccttgtttgccaccatggaacggctcggagatcatcattgcgtaaagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttc

SEQ ID NO: 36 - DNA, 42 bp, synthetic construct
  source 1..42: mol_type = other DNA
  misc_binding 1: bound_moiety = FAM
  misc_binding 42: bound_moiety = BHQ1
aacataatcgaacagctcctcgcccttgctcactagacaatc

SEQ ID NO: 37 - DNA, 42 bp, synthetic construct; identical to SEQ ID NO: 36
  source 1..42: mol_type = other DNA
  misc_binding 1: bound_moiety = FAM
  misc_binding 42: bound_moiety = Super Quencher 1

SEQ ID NO: 38 - DNA, 44 bp, synthetic construct
  source 1..44: mol_type = other DNA
  misc_binding 1: bound_moiety = FAM
  misc_binding 44: bound_moiety = BHQ1
tgctaccatggaacggctcggagatcatcattgcgtaaaggatc

SEQ ID NO: 39 - DNA, 44 bp, synthetic construct; identical to SEQ ID NO: 38
  source 1..44: mol_type = other DNA
  misc_binding 1: bound_moiety = FAM
  misc_binding 44: bound_moiety = Super Quencher 1

SEQ ID NO: 40 - protein, 1112 aa, synthetic construct
MEAGKVGKKGKTNKKFIIRPYLTELNLREDGRLAFQKTFDYMDEQQAALFILGGSVMSHLDESIIRRLGLHKGSKKDLPQRLRVSLHLAIARFRLVSVNYHLDAKAISRMSPTARLAHEQLAEAHRASLIKSPISVWRNRHGVPDEAVHAYLDGNYDPETYAWQDTAMLAKKLCGILKLSPEDFKEASEAMMRNVNFLGCSGSTGSGSSVSNLFGQNEKEDSRNQARIESKTAKVIGKLLESRRPIPMERAVSLVCKSLGHPDAEAAGEDHGGQTDKSTFRQFMRGDYGGSLKELAKKLQKDAHKHRNKSIIPHRETIGAFIKQCASGEFYNKATSESWKDFNAMMNGKYKHNIIFVTEKIALGNAMRKLESNEKAVKASKQLEKLIDKYEMDGNKRFVPKVASVNGLEAYRDVIQDNPQEDDESVKDWLKRLWTTFSEGNDRRLKTPFRWLVESLARLETPESAIKDGCRLMSIRHKHESQRPHPFVRVQSRFTVGDSNIAGSINKPTELKPNRDGRSPEDYWGGNPVVWMSCRLLNGNRWQDMRIPIHNSRYINEVYYTRMGPDGNHALPLKEHARDVKHDYEAETKISRTAARRVNENRMLRRGKPSKRFERVKANSTHNVVFDPKTTASFNRRQDDIYGTINHRHPMVPLAPDGFFAVGGNVIGIDLGESVPLAAGILQKCTSTDSEAVRYACGHWKVVGMGKPGQLLDRQTSANRRKQPHTIIDPMSNMGEPFSSPICQKFIAKCRKFVRAKGSEEDNKAFDDMVKREPSLYSFHGRWGWLLKQMMKAAKGARLDPFREHLEWLLFRTKYGPTNRKSLNLNSMASTKNVISAIDSYMSRRGWKTVEQRQRRDGRLQAARSSLQSNLVNRRLERIKKEESQCIRLAHLFAVTTHLVLEDNLPKQNGASSRADNGRKADWRSRHLAQRLCGIKNDSGSCAIAGVRARRIDPVMTSHMDPFVYSLDNKWAMRARYTKVSLSSMTDYHANLIRSILLEKPGKRQTTEYYQRAVQDFARLHGLDVDEMRKVREARWYKKRIKAKTLYIPCRGGRVFLSTHRLQSDTPHTDSSGAVLWEQDADQVAALNVALRYIDQCYRSNKKGLAKVKKPK

SEQ ID NO: 41 - RNA, 37 nt, synthetic construct
  source 1..37: mol_type = other RNA
actcgactagatttagaatgcccacgatgattgggca

SEQ ID NO: 42 - DNA, 78 bp, synthetic construct
  source 1..78: mol_type = other DNA
tgccaccatggaacggctcggagatcatcattgcgnnnnnnngtgagcaagggcgaggagctgttcaccggggtggtg

SEQ ID NO: 43 - DNA, 78 bp, synthetic construct
  source 1..78: mol_type = other DNA
caccaccccggtgaacagctcctcgcccttgctcacnnnnnnncgcaatgatgatctccgagccgttccatggtggca

(19) United States
(12) Patent Application Publication
(10) Pub. No.: US 2026/0250651 A1
(43) Pub. Date: Aug. 27, 2026

(54) CAS12 PROTEINS AND USES THEREOF

(71) Applicants: GUANGZHOU REFORGENE MEDICINE CO., LTD., Guangzhou (CN); ZHEJIANG SYNSORBIO TECHNOLOGY CO., LTD., Shaoxing (CN); ZHEJIANG SYNSORBIO GENE TECHNOLOGY CO., LTD., Shaoxing (CN)

(72) Inventors: Junbin LIANG, Guangzhou (CN); Liancheng HUANG, Guangzhou (CN); Chongjian CHEN, Shaoxing (CN); Yang SUN, Shaoxing (CN); Weiye PAN, Shaoxing (CN); Hui XU, Guangzhou (CN); Kaiwei SI, Guangzhou (CN); Jinxiu CAI, Guangzhou (CN); Qing LIAO, Guangzhou (CN); Desheng HUANGFU, Guangzhou (CN)

(73) Assignees: GUANGZHOU REFORGENE MEDICINE CO., LTD., Guangzhou (CN); ZHEJIANG SYNSORBIO TECHNOLOGY CO., LTD., Shaoxing (CN); ZHEJIANG SYNSORBIO GENE TECHNOLOGY CO., LTD., Shaoxing (CN)

(21) Appl. No.: 19/457,226

(22) Filed: Jan. 23, 2026

Related U.S. Application Data
(63) Continuation of application No. PCT/CN2024/107663, filed on Jul. 25, 2024.

(30) Foreign Application Priority Data
Jul. 25, 2023 (CN) 202310922104.7
Aug. 18, 2023 (CN) 202311049437.X

(51) Int. Cl. C12N 9/22; C12N 15/11; C12N 15/88

(52) U.S. Cl. CPC C12N 9/226; C12N 15/111; C12N 15/88; C12N 2310/20

(57) ABSTRACT

The present disclosure relates to Cas12 proteins and uses thereof. In certain embodiments, a Cas12 protein is provided whose amino acid sequence has at least 50% sequence identity to SEQ ID NO: 1 and includes one, two, or more amino acid differences relative to SEQ ID NO: 1. In certain embodiments, the Cas12 protein has at least 50% sequence identity to SEQ ID NO: 18. In certain embodiments, the Cas12 protein recognizes a PAM sequence of A. In certain embodiments, gene-editing efficiency of a wild-type Cas12 protein in mammalian cells is improved by introducing rational and irrational amino acid mutations. In certain embodiments, the Cas12 protein exhibits increased editing efficiency and/or a reduced off-target rate and, by virtue of recognizing a single-base A PAM, enables editing of target sequences that were previously difficult to edit, thereby substantially expanding the range of editable targets.

### CROSS-REFERENCE TO RELATED APPLICATIONS

This application is a continuation of International Application No. PCT/CN2024/107663, filed on Jul. 25, 2024, which claims priority to Chinese Patent Application No. 202310922104.7, filed on Jul. 25, 2023, and Chinese Patent Application No. 202311049437.X, filed on Aug. 18, 2023, the entire disclosure of each of which is incorporated herein by reference.

### SEQUENCE LISTING

The present application contains a Sequence Listing submitted electronically in XML format, which is incorporated herein by reference in its entirety. The XML copy was created on Jan. 15, 2026, named “2026 Jan. 15-Sequence Listing-20954-0005US00”, and is 95,270 bytes in size.

### TECHNICAL FIELD

The present disclosure relates to the field of CRISPR gene editing, and more particularly to a Cas12 protein and uses thereof.

### BACKGROUND OF THE INVENTION

The CRISPR-Cas system is an adaptive immune defense formed during the long-term evolution of bacteria and archaea, and can be used to combat invading viruses and foreign DNA. SpCas9 of the CRISPR/Cas9 system derived from Streptococcus pyogenes has been widely used in genetic engineering because it is easy to use and highly efficient. Cas9 is not the only type; in 2015, Cas12 was discovered in bacteria of the genus Acidaminococcus and the family Lachnospiraceae.

An increasing number of Cas12 subtypes have been discovered. However, many researchers in the art are still working to identify new Cas12 proteins.

### SUMMARY

The amino acid sequence of the wild-type Cas12 protein selected in the present disclosure is shown in SEQ ID NO: 1 (1045 aa, from CN111757889B), and rational and non-rational mutations were introduced on that basis.

The present disclosure provides, in one aspect, a Cas12 protein, wherein the amino acid sequence of the Cas12 protein comprises or consists of a sequence having at least 50% sequence identity to SEQ ID NO: 1, and the amino acid sequence of the Cas12 protein comprises or consists of an amino acid sequence that, as compared with SEQ ID NO: 1, has an amino acid difference at one, two, or more positions selected from the following:
N260, N295, T235, D233, S259, Q256, M253, F680, T550, Y668, S246, N229, D678, E875, D166, N325, N168, N884, N369, N879, P605, K872, N456, E601, Q11, N443, D876, E788, G705, V446, S811, E321, E815, A869, V804, N317, N807, H702, V359, K787, P355, K703, V790, L778, D782, N409, D704, D356, T354, M863, L332, Q971, A857, Q262, C567, S849, D590, A933, F962, N930, A794, V58, L475, V61, L526, V469, Q929, L438, N449, L553, K926, T850, 1249, T313, Q450, Y881, R606, Q632, G845, N846, R860, F644, E271, E255, E328, E418, N193, N194, N556, N416, N197, N808, E504, E793, Q186, N812,
N570, P121, E658, L662, 1549, D551, S664, E681, Q294, E225, N663, Y241, W170, S174, M789, S306, C448, I407, K310, C866, I1031, M618, N571 and L484;
the amino acid difference is a substitution of the residue at the position with any other residue, or a deletion of the residue at the position (i.e., the amino acid at the position is absent). The “positions” herein refer to amino acid residue positions of SEQ ID NO: 1.

In specific embodiments of the present disclosure, the amino acid sequence of the Cas12 protein comprises or consists of an amino acid sequence that, as compared with SEQ ID NO: 1, has an amino acid difference at one, two, or more positions selected from the following:
N260, N295, T235, D233, S259, Q256, M253, F680, T550, Y668, S246, N229, D678, E875, D166, N325, N168, N884, N369, N879, P605, K872, N456, E601, Q11, N443, D876, E788, G705, V446, S811, E321, E815, A869, V804, N317, N807, H702, V359, K787, P355, K703, V790, L778, D782, N409, D704, D356, T354, M863, L332, Q971, A857, Q262, C567, S849, D590, A933, F962, N930, A794, V58, L475, V61, L526, V469, Q929, L438, N449, L553, K926, T850, 1249, T313, Q450, Y881, R606, Q632, G845, N846, R860, F644, E271, E255, E328, E418, N193, N194, N556, N416, N197, N808, E504, E793, Q186, N812, N570 and P121.

In specific embodiments of the present disclosure, the amino acid sequence of the Cas12 protein comprises or consists of a sequence having at least 80% sequence identity to SEQ ID NO: 1.

In specific embodiments of the present disclosure, the amino acid sequence of the Cas12 protein comprises or consists of a sequence having at least 85% sequence identity to SEQ ID NO: 1.

In specific embodiments of the present disclosure, the amino acid sequence of the Cas12 protein comprises or consists of a sequence having at least 90%, at least 95%, at least 96%, at least 97%, at least 98%, or at least 99% sequence identity to SEQ ID NO: 1.

In some embodiments of the present disclosure, the Cas12 protein can form a CRISPR complex with a guide polynucleotide. In some embodiments of the present disclosure, the guide polynucleotide guides the CRISPR complex to bind to a target nucleic acid in a sequence-specific manner. In some embodiments of the present disclosure, the guide polynucleotide comprises a guide sequence that is engineered to guide the CRISPR complex to bind to the target nucleic acid in a sequence-specific manner. In some embodiments of the present disclosure, the guide polynucleotide guides the CRISPR complex to bind to and cleave the target nucleic acid in a sequence-specific manner. Optionally, the target nucleic acid is single-stranded nucleic acid or double-stranded nucleic acid; optionally, the target nucleic acid is single-stranded DNA or double-stranded DNA; optionally, cleaving the target nucleic acid refers to cleaving only one strand of a double-stranded nucleic acid, or refers to cleaving both strands of a double-stranded nucleic acid; optionally, cleaving the target nucleic acid refers to cleaving only one strand of a double-stranded DNA, or refers to cleaving both strands of a double-stranded DNA. In some embodiments of the present disclosure, the guide polynucleotide guides the CRISPR complex to bind to the target nucleic acid in a sequence-specific manner, and causes base conversion of at least one base in the target nucleic acid. In some embodiments of the present disclosure, the guide polynucleotide guides the CRISPR complex to bind to the target nucleic acid in a sequence-specific manner, and regulates expression of at least one gene on the target nucleic acid. Optionally, the at least one base is 1 base, 2 bases, 3 bases, 4 bases, 5 bases, 6 bases, 7 bases, 8 bases, 9 bases, or 10 bases. Optionally, the at least one gene is 1 gene, 2 genes, 3 genes, 4 genes, 5 genes, 6 genes, 7 genes, 8 genes, 9 genes, or 10 genes.

In specific embodiments of the present disclosure, a gene-editing efficiency of the Cas12 protein is increased by at least 10% compared with the gene-editing efficiency of the Cas12 protein having the sequence of SEQ ID NO: 1.

In specific embodiments of the present disclosure, the gene-editing efficiency of the Cas12 protein is increased by at least 20%, at least 30%, at least 40%, at least 50%, at least 60%, at least 70%, at least 80%, at least 90%, at least 100%, at least 110%, at least 120%, at least 150%, at least 180%, at least 200%, at least 210%, at least 220%, at least 230%, at least 240%, at least 250%, at least 260%, or at least 270% compared with the gene-editing efficiency of the Cas12 protein having the sequence of SEQ ID NO: 1.

In specific embodiments of the present disclosure, the gene-editing efficiency is the editing efficiency in a reporter system targeted in Example 1 of the present disclosure. In specific embodiments of the present disclosure, the gene-editing efficiency is the editing efficiency in human cells achieved by the Cas12 protein in combination with a gRNA shown in any one of SEQ ID NOs: 10-12. In specific embodiments of the present disclosure, the gene-editing efficiency is the editing efficiency in 293T cells achieved by the Cas12 protein in combination with a gRNA shown in any one of SEQ ID NOs: 10-12. In specific embodiments of the present disclosure, the gene-editing efficiency is the editing efficiency in human cells achieved by the Cas12 protein in combination with a gRNA comprising a guide sequence shown in any one of SEQ ID NOs: 14-16. In specific embodiments of the present disclosure, the gene-editing efficiency is the editing efficiency in 293T cells achieved by the Cas12 protein in combination with a gRNA comprising a guide sequence shown in any one of SEQ ID NOs: 14-16.

In specific embodiments of the present disclosure, the gene-editing efficiency is the efficiency of introducing an indel. In specific embodiments of the present disclosure, the gene-editing efficiency is the single-base editing efficiency of the Cas12 protein, or of the fusion protein or conjugate. In specific embodiments of the present disclosure, the gene-editing efficiency is the efficiency of transcriptional activation or transcriptional repression caused by the Cas12 protein, or by the fusion protein or conjugate. The gene-editing efficiency can be tested by conventional methods in the art.

In specific embodiments of the present disclosure, the amino acid sequence of the Cas12 protein comprises or consists of an amino acid sequence that, as compared with SEQ ID NO: 1, has an amino acid difference at position N260, and further has amino acid difference(s) at positions N295 and/or G705.

In specific embodiments of the present disclosure, the amino acid sequence of the Cas12 protein comprises or consists of an amino acid sequence that, as compared with SEQ ID NO: 1, has amino acid differences at positions N260 and N295, and further

has an amino acid difference at position: E875, D166, N325, N884, N369, N879, P605, K872, N456, D678, E601, Q11, N168, D233, N443, Q450, T313, E788, V446, S811, E321, E815, A869, V804, N317, N807, H702, V359, K787, P355, K703, V790, L778, D782, N409, D704, T235, D356, D876, T354, M863, M789, S306, C448, 1407 or K310; or, alternatively, at residue positions D166 and N168; or at residue positions K872, E875, D876, N879 and N884; or at residue positions T313, N317 and N325.

In specific embodiments of the present disclosure, the amino acid sequence of the Cas12 protein comprises or consists of an amino acid sequence that, as compared with SEQ ID NO: 1, has amino acid differences at positions N260 and N295, and further
has an amino acid difference at position: E875, D166, N325, N884, N369, N879, P605, K872, N456, D678, E601, Q11, N168, D233, N443, Q450, T313, E788, V446, S811, E321, E815, A869, V804, N317, N807, H702, V359, K787, P355, K703, V790, L778, D782, N409, D704, T235, D356, D876, T354, or M863; or, alternatively, at residue positions D166 and N168; or at residue positions K872, E875, D876, N879 and N884.

In specific embodiments of the present disclosure, the amino acid sequence of the Cas12 protein comprises or consists of an amino acid sequence that, as compared with SEQ ID NO: 1, has amino acid differences at positions N260 and G705, and further
has an amino acid difference at position: V446, E788, or S811; or, alternatively, at residue positions D166 and N168.

In specific embodiments of the present disclosure, the amino acid sequence of the Cas12 protein comprises or consists of an amino acid sequence that, compared with SEQ ID NO: 1, has amino acid differences at positions N260, N295, and G705, and further
has an amino acid difference at position: L332, Q971, A857, P355, Q262, C567, S849, D590, A933, F962, N930, A794, N879, K872, N325, V58, L475, V61, N884, N409, L526, V469, Q929, Q11, L438, N369, N449, L553, K926, T850, 1249, T313, T354, N443, N317, Q450, Y881, R606, A869, Q632, G845, N846, R860, F644, C866, 11031, M618, E271, E255, E328, E418, N193, N194, N556, Q256, N416, N197, N808, E504, E793, Q186, N812, N570, N571, P605, P121, N456, N168 or L484; or, at positions V446, E788 and S811.

In specific embodiments of the present disclosure, the amino acid sequence of the Cas12 protein comprises or consists of an amino acid sequence that, compared with SEQ ID NO: 1, has amino acid differences at positions N260, N295, and G705, and further
has an amino acid difference at position: L332, Q971, A857, P355, Q262, C567, S849, D590, A933, F962, N930, A794, N879, K872, N325, V58, L475, V61, N884, N409, L526, V469, Q929, Q11, L438, N369, N449, L553, K926, T850, 1249, T313, T354, N443, N317, Q450, Y881, R606, A869, Q632, G845, N846, R860, F644, C866, I1031, M618, E271, E255, E328, E418, N193, N194, N556, Q256, N416, N197, N808, E504, E793, Q186, N812, N570, N571, P605, P121, N456 or N168; or, alternatively, at residue positions V446, E788 and S811.

In specific embodiments of the present disclosure, the amino acid sequence of the Cas12 protein comprises or consists of an amino acid sequence that, compared with SEQ ID NO: 1, has an amino acid difference at position: N260, N295, T235, D233, S259, Q256, M253, F680, T550, Y668, S246, N229, D678, E658, L662, 1549, D551, S664, E681, Q294, E225, N663, Y241, W170 or S174;
or, alternatively, at residue positions:
N295 and N260;
N295, N260 and E875;
N295, N260 and D166;
N295, N260 and N325;
N295, N260, D166 and N168;
N295, N260 and N884;
N295, N260 and N369;
N295, N260 and N879;
N295, N260 and P605;
N295, N260 and K872;
N295, N260 and N456;
N295, N260 and D678;
N295, N260 and E601;
N295, N260 and Q11;
N295, N260 and N168;
N295, N260 and D233;
N295, N260 and N443;
N295, N260, K872, E875, D876, N879 and N884;
N295, N260 and Q450;
N295, N260, T313, N317 and N325;
N295, N260 and T313;
N295, N260 and E788;
N295, N260 and G705;
N295, N260 and V446;
N295, N260 and S811;
N295, N260 and E321;
N295, N260 and E815;
N295, N260 and A869;
N295, N260 and V804;
N295, N260 and N317;
N295, N260 and N807;
N295, N260 and H702;
N295, N260 and V359;
N295, N260 and K787;
N295, N260 and P355;
N295, N260 and K703;
N295, N260 and V790;
N295, N260 and L778;
N295, N260 and D782;
N295, N260 and N409;
N295, N260 and D704;
N295, N260 and T235;
N295, N260 and D356;
N295, N260 and D876;
N295, N260 and T354;
N295, N260 and M863;
N295, N260 and M789;
N295, N260 and S306;
N295, N260 and C448;
N295, N260 and I407;
N295, N260 and K310;
N295, N260, G705 and L332;
N295, N260, G705 and Q971;
N295, N260, G705 and A857;
N295, N260, G705 and P355;
N295, N260, G705 and Q262;
N295, N260, G705 and C567;
N295, N260, G705 and S849;
N295, N260, G705 and D590;
N295, N260, G705 and A933;
N295, N260, G705 and F962;
N295, N260, G705 and N930;
N295, N260, G705 and A794;
N295, N260, G705 and N879;
N295, N260, G705 and K872;
N295, N260, G705 and N325;
N295, N260, G705 and V58;
N295, N260, G705 and L475;
N295, N260, G705 and V61;
N295, N260, G705 and N884;
N295, N260, G705 and N409;
N295, N260, G705 and L526;
N295, N260, G705 and V469;
N295, N260, G705 and Q929;
N295, N260, G705 and Q11;
N295, N260, G705 and L438;
N295, N260, G705 and N369;
N295, N260, G705 and N449;
N295, N260, G705 and L553;
N295, N260, G705 and K926;
N295, N260, G705 and T850;
N295, N260, G705 and I249;
N295, N260, G705 and T313;
N295, N260, G705 and T354;
N295, N260, G705 and N443;
N295, N260, G705 and N317;
N295, N260, G705 and Q450;
N295, N260, G705 and Y881;
N295, N260, G705 and R606;
N295, N260, G705 and A869;
N295, N260, G705 and Q632;
N295, N260, G705 and G845;
N295, N260, G705 and N846;
N295, N260, G705 and R860;
N295, N260, G705 and F644;
N295, N260, G705 and C866;
N295, N260, G705 and I1031;
N295, N260, G705 and M618;
N260, G705 and E788;
N260, G705, D166 and N168;
N260, G705 and V446;
N260, G705 and S811;
N295, N260, G705 and E271;
N295, N260, G705 and E255;
N295, N260, G705 and E328;
N295, N260, G705 and E418;
N295, N260, G705 and N193;
N295, N260, G705 and N194;
N295, N260, G705 and N556;
N295, N260, G705 and Q256;
N295, N260, G705 and N416;
N295, N260, G705 and N197;
N295, N260, G705 and N808;
N295, N260, G705 and E504;
N295, N260, G705 and E793;
N295, N260, G705 and Q186;
N295, N260, G705 and N812;
N295, N260, G705 and N570;
N295, N260, G705 and N571;
N295, N260, G705, V446, E788 and S811;
N295, N260, E601 and P605;
N295, N260, G705 and P121;
N295, N260, G705 and N456;
N295, N260, G705 and N168; or,
N295, N260, G705 and L484.

In specific embodiments of the present disclosure, the amino acid sequence of the Cas12 protein comprises or consists of an amino acid sequence that, compared with SEQ ID NO: 1, has an amino acid difference at position:
N260, N295, T235, D233, S259, Q256, M253, F680, T550, Y668, S246, N229 or D678;
or, alternatively, at residue positions:
N295 and N260;
N295, N260 and E875;
N295, N260 and D166;
N295, N260 and N325;
N295, N260, D166 and N168;
N295, N260 and N884;
N295, N260 and N369;
N295, N260 and N879;
N295, N260 and P605;
N295, N260 and K872;
N295, N260 and N456;
N295, N260 and D678;
N295, N260 and E601;
N295, N260 and Q11;
N295, N260 and N168;
N295, N260 and D233;
N295, N260 and N443;
N295, N260, K872, E875, D876, N879 and N884;
N295, N260 and E788;
N295, N260 and G705;
N295, N260 and V446;
N295, N260 and S811;
N295, N260 and E321;
N295, N260 and E815;
N295, N260 and A869;
N295, N260 and V804;
N295, N260 and N317;
N295, N260 and N807;
N295, N260 and H702;
N295, N260 and V359;
N295, N260 and K787;
N295, N260 and P355;
N295, N260 and K703;
N295, N260 and V790;
N295, N260 and L778;
N295, N260 and D782;
N295, N260 and N409;
N295, N260 and D704;
N295, N260 and T235;
N295, N260 and D356;
N295, N260 and D876;
N295, N260 and T354;
N295, N260 and M863;
N295, N260, G705 and L332;
N295, N260, G705 and Q971;
N295, N260, G705 and A857;
N295, N260, G705 and P355;
N295, N260, G705 and Q262;
N295, N260, G705 and C567;
N295, N260, G705 and S849;
N295, N260, G705 and D590;
N295, N260, G705 and A933;
N295, N260, G705 and F962;
N295, N260, G705 and N930;
N295, N260, G705 and A794;
N295, N260, G705 and N879;
N295, N260, G705 and K872;
N295, N260, G705 and N325;
N295, N260, G705 and V58;
N295, N260, G705 and L475;
N295, N260, G705 and V61;
N295, N260, G705 and N884;
N295, N260, G705 and N409;
N295, N260, G705 and L526;
N295, N260, G705 and V469;
N295, N260, G705 and Q929;
N295, N260, G705 and Q11;
N295, N260, G705 and L438;
N295, N260, G705 and N369;
N295, N260, G705 and N449;
N295, N260, G705 and L553;
N295, N260, G705 and K926;
N295, N260, G705 and T850;
N295, N260, G705 and I249;
N295, N260, G705 and T313;
N295, N260, G705 and T354;
N295, N260, G705 and N443;
N295, N260, G705 and N317;
N295, N260, G705 and Q450;
N295, N260, G705 and Y881;
N295, N260, G705 and R606;
N295, N260, G705 and A869;
N295, N260, G705 and Q632;
N295, N260, G705 and G845;
N295, N260, G705 and N846;
N295, N260, G705 and R860;
N295, N260, G705 and F644;
N260, G705 and E788;
N260, G705, D166 and N168;
N260, G705 and V446;
N260, G705 and S811;
N295, N260, G705 and E271;
N295, N260, G705 and E255;
N295, N260, G705 and E328;
N295, N260, G705 and E418;
N295, N260, G705 and N193;
N295, N260, G705 and N194;
N295, N260, G705 and N556;
N295, N260, G705 and Q256;
N295, N260, G705 and N416;
N295, N260, G705 and N197;
N295, N260, G705 and N808;
N295, N260, G705 and E504;
N295, N260, G705 and E793;
N295, N260, G705 and Q186;
N295, N260, G705 and N812;
N295, N260, G705 and N570;
N295, N260, G705, V446, E788 and S811;
N295, N260, E601 and P605;
N295, N260, G705 and P121;
N295, N260, G705 and N456; or,
N295, N260, G705 and N168.

In specific embodiments of the present disclosure, the amino acid difference is:
substitutions at positions N260, N295, T235, D233, S259, Q256, M253, F680, T550, Y668, S246, N229, E875, D166, P605, E601, D876, E788, G705, V446, S811, E321, E815, A869, V804, N807, H702, V359, K787, K703, V790, L778, D782, D704, D356, M863, C567, D590, N930, A794, V58, L475, V469, L438, L553, Y881, R606, E271, E255, E328, E418, N193, N194, N556, Q256, N416, N197, N808, E504, E793, Q186, N812, L553, N570, L475, P121, E658, L662, 1549, D551, S664, E681, Q294, E225, N663, Y241, W170, S174, M789, S306, C448, 1407, K310, 11031, N571 or L484 with positively charged amino acids, such as R, H, or K; and/or,
a substitution at positions Q632 or N846 with negatively charged amino acids, such as D or E; and/or,
a substitution at positions D678, P355, Q262, Q971, A933, F962, N879, L332, N325, V61, N884, N409, L526, Q11, S849, A857, Q929, N369, K926, T313, T354, N443, N317, T850, Q450, N456, N168 or N449 with positively charged amino acids, such as R, H, or K; or changes to negatively charged amino acids, such as D or E; and/or,
a substitution at positions 1249 or F644 with positively charged amino acids, such as R, H, or K; or substitutions with negatively charged amino acids, such as D or E; or substitutions with nonpolar amino acids, such as G, P, A, I, L, V, M, F, W, or Y; and/or,
a substitution at position K872 with positively charged amino acids, such as R, H, or K; or substitutions with negatively charged amino acids, such as D or E; or substitutions with neutral amino acids, such as N, C, Q, S, or T; and/or,
a substitution at positions A869, C866, or M618 with nonpolar amino acids, such as G, P, A, I, L, V, M, F, W, or Y; and/or,
position R860 is substituted with neutral amino acids, such asN, C, Q, S or T; and/or,
the amino acid at position G845 is deleted (G845A).

In specific embodiments of the present disclosure, the amino acid sequence of the Cas12 protein comprises or consists of an amino acid sequence that, as compared with SEQ ID NO: 1, has one, two, or more amino acid differences selected from the following:
N260R, N295R, T235R, D233R, S259R, Q256R, M253R, F680R, T550R, Y668R, S246R, N229R, D678R, E875R, D166R, N325R, N168R, N884R, N369R, N879R, P605R, K872R, N456R, D678E, E601R, Q11R, N443R, D876R, E788R, G705R, V446R, S811R, E321R, E815R, A869R, V804R, N317R, N807R, H702R, V359R, K787R, P355R, K703R, V790R, L778R, D782R, N409R, D704R, D356R, T354R, M863R, L332R, Q971R, A857R, P355E, Q262E, Q971E, C567R, S849R, D590R, A933E, F962E, N930R, A794R, N879E, L332E, F962R, K872E, N325E, V58R, L475R, V61E, N884E, N409E, L526E, V469R, L526R, Q929R, Q11E, S849E, L438R, A857E, A933R, Q929E, N369E, N449R, L553R, Q262R, K926E, T850R, I249F, T313E, I249R, T354E, I249E, N443E, N317E, T850E, Q450E, K872N, Y881H, R606K, A869G, Q632E, G845A, N846D, R860S, F644Y, E271K, E255K, E328K, E418K, N193K, N194K, N556K, Q262K, Q256K, N416K, N197K, N808K, E504K, E793K, Q186K, N812K, L553K, N570K, L475K, V61R, P121R, N456E, N168E, N449E, K926R, E658R, L662R, 1549R, D551R, S664R, E681R, Q294R, E225R, N663R, Y241R, W170R, S174R, Q450R, T313R, M789R, S306R, C448R, 1407R, K310R, C866L, 11031K, M618Y, N571K, L484R, F644E or F644R.

In specific embodiments of the present disclosure, the amino acid sequence of the Cas12 protein comprises or consists of an amino acid sequence that, as compared with SEQ ID NO: 1, has one, two, or more amino acid differences selected from the following:
N260R, N295R, T235R, D233R, S259R, Q256R, M253R, F680R, T550R, Y668R, S246R, N229R, D678R, E875R, D166R, N325R, N168R, N884R, N369R, N879R, P605R, K872R, N456R, D678E, E601R, Q11R, N443R, D876R, E788R, G705R, V446R, S811R, E321R, E815R, A869R, V804R, N317R, N807R, H702R, V359R, K787R, P355R, K703R, V790R, L778R, D782R, N409R, D704R, D356R, T354R, M863R, L332R, Q971R, A857R, P355E, Q262E, Q971E, C567R, S849R, D590R, A933E, F962E, N930R, A794R, N879E, L332E, F962R, K872E, N325E, V58R, L475R, V61E, N884E, N409E, L526E, V469R, L526R, Q929R, Q11E, S849E, L438R, A857E, A933R, Q929E, N369E, N449R, L553R, Q262R, K926E, T850R, I249F, T313E, I249R, T354E, I249E, N443E, N317E, T850E, Q450E, K872N, Y881H, R606K, A869G, Q632E, G845A, N846D, R860S, F644Y, E271K, E255K, E328K, E418K, N193K, N194K, N556K, Q262K, Q256K, N416K, N197K, N808K, E504K, E793K, Q186K, N812K, L553K, N570K, L475K, V61R, P121R, N456E, N168E, N449E or K926R.

In specific embodiments of the present disclosure, the amino acid sequence of the Cas12 protein comprises or consists of an amino acid sequence that, compared with SEQ ID NO: 1, has amino acid difference of N260R; and optionally, further has amino acid differences of N295R and/or G705R.

In specific embodiments of the present disclosure, the amino acid sequence of the Cas12 protein comprises or consists of an amino acid sequence that, compared with SEQ ID NO: 1, has amino acid differences of N260R and N295R, and
further has an amino acid difference selected from the following: E875R, D166R, N325R, N884R, N369R, N879R, P605R, K872R, N456R, D678E, E601R, Q11R, N168R, D233R, N443R, Q450R, T313R, E788R, V446R, S811R, E321R, E815R, A869R, V804R, N317R, N807R, H702R, V359R, K787R, P355R, K703R, V790R, L778R, D782R, N409R, D704R, T235R, D356R, D876R, T354R, M863R, M789R, S306R, C448R, I407R or K310R; or alternatively, has amino acid differences of D166R or N168R; or has amino acid differences of K872R, E875R, D876R, N879R and N884R; or has amino acid differences of T313R, N317R and N325R.

In specific embodiments of the present disclosure, the amino acid sequence of the Cas12 protein comprises or consists of an amino acid sequence that, compared with SEQ ID NO: 1, has amino acid differences of N260R and N295R, and
further has an amino acid difference selected from the following: E875R, D166R, N325R, N884R, N369R, N879R, P605R, K872R, N456R, D678E, E601R, Q11R, N168R, D233R, N443R, E788R, V446R, S811R, E321R, E815R, A869R, V804R, N317R, N807R, H702R, V359R, K787R, P355R, K703R, V790R, L778R, D782R, N409R, D704R, T235R, D356R, D876R, T354R or M863R; or alternatively, has amino acid differences of D166R and N168R; or has amino acid differences of K872R, E875R, D876R, N879R and N884R.

In specific embodiments of the present disclosure, the amino acid sequence of the Cas12 protein comprises or consists of an amino acid sequence that, compared with SEQ ID NO: 1, has amino acid differences of N260R and G705R, and
further has amino acid differences of: V446R, E788R or S811R; or, D166R and N168R.

In specific embodiments of the present disclosure, the amino acid sequence of the Cas12 protein comprises or consists of an amino acid sequence that, compared with SEQ ID NO: 1, has amino acid differences of N260R, N295R, and G705R, and
further has an amino acid difference of L332R, Q971R, A857R, P355E, Q262E, Q971E, C567R, S849R, D590R, A933E, F962E, N930R, A794R, N879E, L332E, F962R, K872E, N325E, V58R, L475R, V61E, N884E, N409E, L526E, V469R, L526R, Q929R, Q11E, S849E, L438R, A857E, A933R, Q929E, N369E, N449R, L553R, Q262R, K926E, T850R, I249F, T313E, I249R, T354E, I249E, N443E, N317E, T850E, Q450E, K872N, Y881H, R606K, A869G, Q632E, G845A, N846D, R860S, F644Y, C866L, I1031K, M618Y, E271K, E255K, E328K, E418K, N193K, N194K, N556K, Q262K, Q256K, N416K, N197K, N808K, E504K, E793K, Q186K, N812K, L553K, N570K, L475K, N571K, V61R, P605R, P121R, N456E, N168E, N449E, K926R, L484R, F644E or F644R; or alternatively, has amino acid differences of V446R, E788R and S811R.

In specific embodiments of the present disclosure, the amino acid sequence of the Cas12 protein comprises or consists of an amino acid sequence that, compared with SEQ ID NO: 1, has amino acid differences of N260R, N295R, and G705R, and
further has an amino acid difference of: L332R, Q971R, A857R, P355E, Q262E, Q971E, C567R, S849R, D590R, A933E, F962E, N930R, A794R, N879E, L332E, F962R, K872E, N325E, V58R, L475R, V61E, N884E, N409E, L526E, V469R, L526R, Q929R, Q11E, S849E, L438R, A857E, A933R, Q929E, N369E, N449R, L553R, Q262R, K926E, T850R, I249F, T313E, I249R, T354E, I249E, N443E, N317E, T850E, Q450E, K872N, Y881H, R606K, A869G, Q632E, G845A, N846D, R860S, F644Y, E271K, E255K, E328K, E418K, N193K, N194K, N556K, Q262K, Q256K, N416K, N197K, N808K, E504K, E793K, Q186K, N812K, L553K, N570K, L475K, V61R, P605R, P121R, N456E, N168E, N449E or K926R; or alternatively, has amino acid differences of V446R, E788R and S811R.

In specific embodiments of the present disclosure, as compared with SEQ ID NO: 1, the amino acid difference is: N260R, N295R, T235R, D233R, S259R, Q256R, M253R, F680R, T550R, Y668R, S246R, N229R, D678R, E658R, L662R, 1549R, D551R, S664R, E681R, Q294R, E225R, N663R, Y241R, W170R, or S174R; or
the amino acid differences are:
N295R and N260R;
N295R, N260R and E875R;
N295R, N260R and D166R;
N295R, N260R and N325R;
N295R, N260R, D166R and N168R;
N295R, N260R and N884R;
N295R, N260R and N369R;
N295R, N260R and N879R;
N295R, N260R and P605R;
N295R, N260R and K872R;
N295R, N260R and N456R;
N295R, N260R and D678E;
N295R, N260R and E601R;
N295R, N260R and Q11R;
N295R, N260R and N168R;
N295R, N260R and D233R;
N295R, N260R and N443R;
N295R, N260R, K872R, E875R, D876R, N879R and N884R;
N295R, N260R and Q450R;
N295R, N260R, T313R, N317R and N325R;
N295R, N260R and T313R;
N295R, N260R and E788R;
N295R, N260R and G705R;
N295R, N260R and V446R;
N295R, N260R and S811R;
N295R, N260R and E321R;
N295R, N260R and E815R;
N295R, N260R and A869R;
N295R, N260R and V804R;
N295R, N260R and N317R;
N295R, N260R and N807R;
N295R, N260R and H702R;
N295R, N260R and V359R;
N295R, N260R and K787R;
N295R, N260R and P355R;
N295R, N260R and K703R;
N295R, N260R and V790R;
N295R, N260R and L778R;
N295R, N260R and D782R;
N295R, N260R and N409R;
N295R, N260R and D704R;
N295R, N260R and T235R;
N295R, N260R and D356R;
N295R, N260R and D876R;
N295R, N260R and T354R;
N295R, N260R and M863R;
N295R, N260R and M789R;
N295R, N260R and S306R;
N295R, N260R and C448R;
N295R, N260R and I407R;
N295R, N260R and K310R;
N295R, N260R, G705R and L332R;
N295R, N260R, G705R and Q971R;
N295R, N260R, G705R and A857R;
N295R, N260R, G705R and P355E;
N295R, N260R, G705R and Q262E;
N295R, N260R, G705R and Q971E;
N295R, N260R, G705R and C567R;
N295R, N260R, G705R and S849R;
N295R, N260R, G705R and D590R;
N295R, N260R, G705R and A933E;
N295R, N260R, G705R and F962E;
N295R, N260R, G705R and N930R;
N295R, N260R, G705R and A794R;
N295R, N260R, G705R and N879E;
N295R, N260R, G705R and L332E;
N295R, N260R, G705R and F962R;
N295R, N260R, G705R and K872E;
N295R, N260R, G705R and N325E;
N295R, N260R, G705R and V58R;
N295R, N260R, G705R and L475R;
N295R, N260R, G705R and V61E;
N295R, N260R, G705R and N884E;
N295R, N260R, G705R and N409E;
N295R, N260R, G705R and L526E;
N295R, N260R, G705R and V469R;
N295R, N260R, G705R and L526R;
N295R, N260R, G705R and Q929R;
N295R, N260R, G705R and Q11E;
N295R, N260R, G705R and S849E;
N295R, N260R, G705R and L438R;
N295R, N260R, G705R and A857E;
N295R, N260R, G705R and A933R;
N295R, N260R, G705R and Q929E;
N295R, N260R, G705R and N369E;
N295R, N260R, G705R and N449R;
N295R, N260R, G705R and L553R;
N295R, N260R, G705R and Q262R;
N295R, N260R, G705R and K926E;
N295R, N260R, G705R and T850R;
N295R, N260R, G705R and I249F;
N295R, N260R, G705R and T313E;
N295R, N260R, G705R and I249R;
N295R, N260R, G705R and T354E;
N295R, N260R, G705R and I249E;
N295R, N260R, G705R and N443E;
N295R, N260R, G705R and N317E;
N295R, N260R, G705R and T850E;
N295R, N260R, G705R and Q450E;
N295R, N260R, G705R and K872N;
N295R, N260R, G705R and Y881H;
N295R, N260R, G705R and R606K;
N295R, N260R, G705R and A869G;
N295R, N260R, G705R and Q632E;
N295R, N260R, G705R and G845A;
N295R, N260R, G705R and N846D;
N295R, N260R, G705R and R860S;
N295R, N260R, G705R and F644Y;
N295R, N260R, G705R and C866L;
N295R, N260R, G705R and I1031K;
N295R, N260R, G705R and M618Y;
N260R, G705R and E788R;
N260R, G705R, D166R and N168R;
N260R, G705R and V446R;
N260R, G705R and S811R;
N295R, N260R, G705R and E271K;
N295R, N260R, G705R and E255K;
N295R, N260R, G705R and E328K;
N295R, N260R, G705R and E418K;
N295R, N260R, G705R and N193K;
N295R, N260R, G705R and N194K;
N295R, N260R, G705R and N556K;
N295R, N260R, G705R and Q262K;
N295R, N260R, G705R and Q256K;
N295R, N260R, G705R and N416K;
N295R, N260R, G705R and N197K;
N295R, N260R, G705R and N808K;
N295R, N260R, G705R and E504K;
N295R, N260R, G705R and E793K;
N295R, N260R, G705R and Q186K;
N295R, N260R, G705R and N812K;
N295R, N260R, G705R and L553K;
N295R, N260R, G705R and N570K;
N295R, N260R, G705R and L475K;
N295R, N260R, G705R and N571K;
N295R, N260R, G705R and V61R;
N295R, N260R, G705R, V446R, E788R and S811R;
N295R, N260R, E601R and P605R;
N295R, N260R, G705R and P121R;
N295R, N260R, G705R and N456E;
N295R, N260R, G705R and N168E;
N295R, N260R, G705R and N449E;
N295R, N260R, G705R and K926R;
N295R, N260R, G705R and L484R;
N295R, N260R, G705R and F644E; or,
N295R, N260R, G705R and F644R.

In specific embodiments of the present disclosure, as compared with SEQ ID NO: 1, the amino acid difference is: N260R, N295R, T235R, D233R, S259R, Q256R, M253R, F680R, T550R, Y668R, S246R, N229R, or D678R; or
the amino acid differences are:
N295R and N260R;
N295R, N260R and E875R;
N295R, N260R and D166R;
N295R, N260R and N325R;
N295R, N260R, D166R and N168R;
N295R, N260R and N884R;
N295R, N260R and N369R;
N295R, N260R and N879R;
N295R, N260R and P605R;
N295R, N260R and K872R;
N295R, N260R and N456R;
N295R, N260R and D678E;
N295R, N260R and E601R;
N295R, N260R and Q11R;
N295R, N260R and N168R;
N295R, N260R and D233R;
N295R, N260R and N443R;
N295R, N260R, K872R, E875R, D876R, N879R and N884R;
N295R, N260R and E788R;
N295R, N260R and G705R;
N295R, N260R and V446R;
N295R, N260R and S811R;
N295R, N260R and E321R;
N295R, N260R and E815R;
N295R, N260R and A869R;
N295R, N260R and V804R;
N295R, N260R and N317R;
N295R, N260R and N807R;
N295R, N260R and H702R;
N295R, N260R and V359R;
N295R, N260R and K787R;
N295R, N260R and P355R;
N295R, N260R and K703R;
N295R, N260R and V790R;
N295R, N260R and L778R;
N295R, N260R and D782R;
N295R, N260R and N409R;
N295R, N260R and D704R;
N295R, N260R and T235R;
N295R, N260R and D356R;
N295R, N260R and D876R;
N295R, N260R and T354R;
N295R, N260R and M863R;
N295R, N260R, G705R and L332R;
N295R, N260R, G705R and Q971R;
N295R, N260R, G705R and A857R;
N295R, N260R, G705R and P355E;
N295R, N260R, G705R and Q262E;
N295R, N260R, G705R and Q971E;
N295R, N260R, G705R and C567R;
N295R, N260R, G705R and S849R;
N295R, N260R, G705R and D590R;
N295R, N260R, G705R and A933E;
N295R, N260R, G705R and F962E;
N295R, N260R, G705R and N930R;
N295R, N260R, G705R and A794R;
N295R, N260R, G705R and N879E;
N295R, N260R, G705R and L332E;
N295R, N260R, G705R and F962R;
N295R, N260R, G705R and K872E;
N295R, N260R, G705R and N325E;
N295R, N260R, G705R and V58R;
N295R, N260R, G705R and L475R;
N295R, N260R, G705R and V61E;
N295R, N260R, G705R and N884E;
N295R, N260R, G705R and N409E;
N295R, N260R, G705R and L526E;
N295R, N260R, G705R and V469R;
N295R, N260R, G705R and L526R;
N295R, N260R, G705R and Q929R;
N295R, N260R, G705R and Q11E;
N295R, N260R, G705R and S849E;
N295R, N260R, G705R and L438R;
N295R, N260R, G705R and A857E;
N295R, N260R, G705R and A933R;
N295R, N260R, G705R and Q929E;
N295R, N260R, G705R and N369E;
N295R, N260R, G705R and N449R;
N295R, N260R, G705R and L553R;
N295R, N260R, G705R and Q262R;
N295R, N260R, G705R and K926E;
N295R, N260R, G705R and T850R;
N295R, N260R, G705R and I249F;
N295R, N260R, G705R and T313E;
N295R, N260R, G705R and I249R;
N295R, N260R, G705R and T354E;
N295R, N260R, G705R and I249E;
N295R, N260R, G705R and N443E;
N295R, N260R, G705R and N317E;
N295R, N260R, G705R and T850E;
N295R, N260R, G705R and Q450E;
N295R, N260R, G705R and K872N;
N295R, N260R, G705R and Y881H;
N295R, N260R, G705R and R606K;
N295R, N260R, G705R and A869G;
N295R, N260R, G705R and Q632E;
N295R, N260R, G705R and G845A;
N295R, N260R, G705R and N846D;
N295R, N260R, G705R and R860S;
N295R, N260R, G705R and F644Y;
N260R, G705R and E788R;
N260R, G705R, D166R and N168R;
N260R, G705R and V446R;
N260R, G705R and S811R;
N295R, N260R, G705R and E271K;
N295R, N260R, G705R and E255K;
N295R, N260R, G705R and E328K;
N295R, N260R, G705R and E418K;
N295R, N260R, G705R and N193K;
N295R, N260R, G705R and N194K;
N295R, N260R, G705R and N556K;
N295R, N260R, G705R and Q262K;
N295R, N260R, G705R and Q256K;
N295R, N260R, G705R and N416K;
N295R, N260R, G705R and N197K;
N295R, N260R, G705R and N808K;
N295R, N260R, G705R and E504K;
N295R, N260R, G705R and E793K;
N295R, N260R, G705R and Q186K;
N295R, N260R, G705R and N812K;
N295R, N260R, G705R and L553K;
N295R, N260R, G705R and N570K;
N295R, N260R, G705R and L475K;
N295R, N260R, G705R and V61R;
N295R, N260R, G705R, V446R, E788R and S811R;
N295R, N260R, E601R and P605R;
N295R, N260R, G705R and P121R;
N295R, N260R, G705R and N456E;
N295R, N260R, G705R and N168E;
N295R, N260R, G705R and N449E; or,
N295R, N260R, G705R and K926R.

Optionally, the Cas12 protein can recognize a 5′-TTN PAM sequence, wherein N is A, T, C, or G.

In some embodiments, the Cas12 protein can recognize a 5′-TTA PAM sequence. In some embodiments, the Cas12 protein can recognize a 5′-TTT PAM sequence. In some embodiments, the Cas12 protein can recognize a 5′-TTC PAM sequence. In some embodiments, the Cas12 protein can recognize a 5′-TTG PAM sequence.

The present disclosure provides, in another aspect, a fusion protein or conjugate, wherein the fusion protein or conjugate comprises a Cas12 protein as described in the present disclosure, or a functional fragment thereof, fused to a homologous or heterologous functional domain.

In some embodiments, fusion of the Cas12 protein does not alter the original functions of the Cas12 protein, including but not limited to binding to and cleaving a target nucleic acid.

In specific embodiments of the present disclosure, the homologous or heterologous functional domain is optionally selected from one or more of the following: a subcellular localization signal, a DNA-binding domain, a protein targeting moiety, a transcriptional activation domain, a transcriptional repression domain, a nuclease, a base-editing domain (e.g., a deaminase domain), a methyltransferase, a demethylase, a transcription release factor, a histone deacetylase, a polypeptide having ssDNA cleavage activity, a polypeptide having dsDNA cleavage activity, a DNA ligase, an epitope tag, a reporter protein, and a detectable label.

In specific embodiments of the present disclosure, the Cas12 protein is covalently linked to the homologous or heterologous functional domain.

In specific embodiments of the present disclosure, the Cas12 protein is directly linked to the homologous or heterologous functional domain, or is covalently linked to the homologous or heterologous functional domain via an amino acid linker or a non-amino acid linker.

In specific embodiments of the present disclosure, the homologous or heterologous functional domain is fused or conjugated to the N-terminus, the C-terminus, or an internal region of the Cas12 protein.

Optionally, the fusion protein or conjugate can recognize a 5′-TTN PAM sequence, wherein N is A, T, C, or G.

The present disclosure provides, in one aspect, an isolated nucleic acid encoding the Cas12 protein as described in the present disclosure, or the fusion protein or conjugate as described in the present disclosure.

In specific embodiments of the present disclosure, the nucleic acid is codon optimized for expression in cells.

In specific embodiments of the present disclosure, the nucleic acid is codon optimized for expression in a eukaryote, a mammal such as a human or a non-human mammal, a plant, an insect, a bird, a reptile, a rodent (e.g., a mouse or a rat), a fish, a worm/nematode, or yeast.

The present disclosure provides, in one aspect, a CRISPR-Cas12 system, comprising:
a. the Cas12 protein as described in the present disclosure, the fusion protein or conjugate as described in the present disclosure, or the nucleic acid as described in the present disclosure;
and
b. a guide polynucleotide, or a polynucleotide sequence encoding the guide polynucleotide;
wherein the Cas12 protein or the fusion protein or conjugate forms a CRISPR complex with the guide polynucleotide; and the guide polynucleotide comprises a guide sequence that is engineered to guide the CRISPR complex to bind to a target nucleic acid in a sequence-specific manner.

In specific embodiments of the present disclosure, the guide polynucleotide comprises a direct repeat sequence linked to the guide sequence; and the nucleotide sequence of the direct repeat sequence has at least 80% sequence identity to SEQ ID NO: 17.

In specific embodiments of the present disclosure, the nucleotide sequence of the direct repeat sequence is as shown in SEQ ID NO: 17.

In specific embodiments of the present disclosure, the target nucleic acid is DNA or RNA, preferably dsDNA or ssDNA.

In specific embodiments of the present disclosure, the DNA is eukaryotic DNA; preferably, the eukaryotic DNA is non-human mammalian DNA, non-human primate DNA, human DNA, plant DNA, insect DNA, avian DNA, reptile DNA, rodent DNA, fish DNA, worm/nematode DNA, or yeast DNA.

In specific embodiments of the present disclosure, the target nucleic acid is a disease-related gene or a gene related to a signal transduction biochemical pathway, or the target nucleic acid is a reporter gene.

In specific embodiments of the present disclosure, the disease-related gene or the gene related to the signal transduction biochemical pathway is a TTR (transthyretin), HBB (beta-globin), or HBG (gamma-globin) gene; and the reporter gene is a GFP (green fluorescent protein) gene.

In specific embodiments of the present disclosure, the guide sequence comprises 15-35 nucleotides; and/or the guide sequence hybridizes to the target nucleic acid, wherein the guide sequence is 90%-100% complementary to the target nucleic acid, preferably with no more than one nucleotide mismatch. In specific embodiments of the present disclosure, the guide sequence is optionally selected from the sequences shown in SEQ ID NOs: 14-16.

In specific embodiments of the present disclosure, the guide sequence is located at the 3′ end of the direct repeat sequence.

The present disclosure provides, in one aspect, a vector system, wherein the vector system comprises one or more vectors, and the vector comprises the isolated nucleic acid as described in the present disclosure, or the CRISPR-Cas12 system as described in the present disclosure.

In specific embodiments of the present disclosure, the vector further comprises a regulatory sequence.

In specific embodiments of the present disclosure, the regulatory sequence comprises one or more selected from: a promoter, an enhancer, an internal ribosome entry site, and a transcription termination signal; the promoter is, for example, a constitutive promoter, an inducible promoter, a ubiquitous promoter, or a tissue-specific promoter; and/or the transcription termination signal is, for example, a polyadenylation signal or a poly(U) sequence.

In specific embodiments of the present disclosure, the regulatory sequence is operably linked to the vector.

In specific embodiments of the present disclosure, the backbone of the vector is pCDNA3.1.

In specific embodiments of the present disclosure, the vector is an adeno-associated virus vector, a lentiviral vector, a ribonucleoprotein complex, or a virus-like particle.

In specific embodiments of the present disclosure:
when the vector is an adeno-associated virus vector, the adeno-associated virus vector is a recombinant adeno-associated virus vector of serotype AAV1, AAV2, AAV4, AAV5, AAV6, AAV7, AAVrh74, AAV8, AAV9, AAV10, AAV11, AAV12, or AAV13;
when the vector is a lentiviral vector, the lentiviral vector is pseudotyped with an envelope protein;
optionally, the isolated nucleic acid is linked to an aptamer sequence;
when the vector is a virus-like particle, the isolated nucleic acid is linked to a gene encoding a gag protein.

The present disclosure provides, in one aspect, a delivery system, wherein the delivery system comprises:
(1) a delivery vehicle, and
(2) the Cas12 protein as described in the present disclosure, the fusion protein or conjugate as described in the present disclosure, or the nucleic acid as described in the present disclosure; the CRISPR-Cas12 system as described in the present disclosure; or the vector system as described in the present disclosure.

In specific embodiments of the present disclosure, the delivery vehicle is a lipid nanoparticle, a nanoparticle, a liposome, an exosome, a microbubble, or a gene gun.

In specific embodiments of the present disclosure, the delivery vehicle is a lipid nanoparticle, wherein the lipid nanoparticle comprises the guide polynucleotide and an mRNA encoding the Cas12 protein or the fusion protein or conjugate.

The present disclosure provides, in one aspect, a cell, wherein the cell comprises the Cas12 protein as described in the present disclosure, the fusion protein or conjugate as described in the present disclosure, the isolated nucleic acid as described in the present disclosure, the CRISPR-Cas12 system as described in the present disclosure, or the vector system as described in the present disclosure.

In specific embodiments of the present disclosure, the cell is a eukaryotic cell.

In specific embodiments of the present disclosure, the eukaryotic cell is a mammalian cell.

The present disclosure provides, in one aspect, a pharmaceutical composition, wherein the pharmaceutical composition comprises the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, or the cell as described in the present disclosure.

In specific embodiments of the present disclosure, the pharmaceutical composition comprises a pharmaceutically acceptable excipient.

The present disclosure provides, in one aspect, a kit, wherein the kit comprises the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, or the cell as described in the present disclosure.

The present disclosure provides, in one aspect, use of the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure in the preparation of a reagent or medicament for diagnosing, treating, and/or preventing a disease or condition associated with a target nucleic acid.

In specific embodiments of the present disclosure, the reagent or medicament is used for one or more of the following: cleaving one or more target nucleic acid molecules or nicking one or more target nucleic acid molecules; activating or upregulating expression of one or more target nucleic acid molecules; activating or inhibiting transcription of one or more target nucleic acid molecules; inactivating one or more target nucleic acid molecules; visualizing, labeling, or detecting one or more target nucleic acid molecules; binding to one or more target nucleic acid molecules; transporting one or more target nucleic acid molecules; and masking one or more target nucleic acid molecules.

The present disclosure provides, in one aspect, a method for detecting, binding to, or cleaving a target nucleic acid, wherein the method comprises contacting the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure with the target nucleic acid.

In specific embodiments of the present disclosure, the method is a method for non-diagnostic and/or non-therapeutic purposes; and/or the fusion protein or conjugate comprises a detectable label, such as a label detectable by fluorescence, DNA blotting, or FISH.

The present disclosure provides, in one aspect, a method for altering a cell state, wherein the method comprises contacting a cell with the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure, thereby altering the cell state.

In specific embodiments of the present disclosure, the method results in one or more of the following: (i) inducing cellular senescence in vitro or in vivo; (ii) cell cycle arrest in vitro or in vivo; (iii) inhibition of cell growth and/or inhibition of cell proliferation in vitro or in vivo; (iv) inducing anergy in vitro or in vivo; (v) inducing apoptosis in vitro or in vivo; and (vi) inducing necrosis in vitro or in vivo.

In specific embodiments of the present disclosure, the method is a method for non-diagnostic and/or non-therapeutic purposes.

The present disclosure provides, in one aspect, a method for diagnosing, treating, and/or preventing a disease or condition associated with a target nucleic acid, wherein the method comprises administering, to a sample from a subject in need thereof or to a subject in need thereof, the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure.

The present disclosure provides, in one aspect, the Cas12 protein as described in the present disclosure, the fusion protein or conjugate as described in the present disclosure, the isolated nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure, for use in diagnosing, treating, and/or preventing a disease or condition associated with a target nucleic acid.

The present disclosure provides Cas12 proteins and uses thereof.

In one aspect, the present disclosure provides a Cas12 protein, wherein the amino acid sequence of the Cas12 protein comprises or consists of a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.93%, at least 98.94%, at least 98.95%, at least 98.96%, at least 98.97%, at least 98.98%, at least 98.99%, at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 18.

In specific embodiments of the present disclosure, the Cas12 protein recognizes a PAM sequence of A.

In specific embodiments of the present disclosure, the amino acid sequence of the Cas12 protein comprises or consists of a sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 98.93%, at least 98.94%, at least 98.95%, at least 98.96%, at least 98.97%, at least 98.98%, at least 98.99%, at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 18, and the Cas12 protein recognizes a PAM sequence of A.

In preferred embodiments of the present disclosure, the Cas12 protein does not include a Cas12 protein having at least 70% sequence identity to SEQ ID NO: 40 and recognizing a PAM sequence other than A.

In preferred embodiments of the present disclosure, the Cas12 protein does not include a Cas12 protein having at least 80%, at least 85%, at least 90%, at least 91%, at least 92%, at least 93%, at least 94%, at least 95%, at least 96%, at least 97%, at least 98%, at least 99.0%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 40 and recognizing a PAM sequence other than A.

In specific embodiments of the present disclosure, the at least 50% identity is at least 70%, at least 80%, at least 85%, at least 90%, at least 95%, at least 98%, or at least 99% identity.

In specific embodiments of the present disclosure, the Cas12 protein retains the function of the protein shown in SEQ ID NO: 18.

In specific embodiments of the present disclosure, the Cas12 protein can form a complex with a guide polynucleotide. In specific embodiments of the present disclosure, the Cas12 protein can bind to a target nucleic acid in a sequence-specific manner with a guide polynucleotide.

In specific embodiments of the present disclosure, the Cas12 protein can form a complex with a guide polynucleotide. In specific embodiments of the present disclosure, the Cas12 protein can bind to a target DNA in a sequence-specific manner with a guide polynucleotide.

In specific embodiments of the present disclosure, the Cas12 protein can specifically bind to and cleave a target nucleic acid with a guide polynucleotide. In specific embodiments of the present disclosure, the Cas12 protein can specifically bind to and cleave a target DNA with a guide polynucleotide. In specific embodiments of the present disclosure, the Cas12 protein can form a complex with a guide polynucleotide, and the complex can specifically bind to and cleave a target nucleic acid. In specific embodiments of the present disclosure, the Cas12 protein can form a complex with a guide polynucleotide, and the complex can specifically bind to and cleave a target DNA.

As used herein, retaining the function of the protein shown in SEQ ID NO: 18 means retaining the ability to form a complex with a guide polynucleotide, retaining the ability to bind to a target nucleic acid that is complementary to the guide sequence of the guide polynucleotide, retaining the ability to cleave the target nucleic acid when guided by the guide polynucleotide, and/or retaining the ability to process an RNA transcript of the guide sequence into a guide polynucleotide molecule.

In specific embodiments of the present disclosure, retaining the function of the protein shown in SEQ ID NO: 18 refers to retaining the ability to form a complex with a guide polynucleotide.

In specific embodiments of the present disclosure, retaining the function of the protein shown in SEQ ID NO: 18 refers to retaining the ability to bind to a target nucleic acid that is complementary to the guide sequence of the guide polynucleotide.

In specific embodiments of the present disclosure, retaining the function of the protein shown in SEQ ID NO: 18 refers to retaining the ability to cleave a target nucleic acid when guided by a guide polynucleotide.

In specific embodiments of the present disclosure, retaining the function of the protein shown in SEQ ID NO: 18 refers to retaining the ability to process an RNA transcript of the guide sequence into a guide polynucleotide molecule.

In preferred embodiments of the present disclosure, the amino acid sequence of the Cas12 protein comprises or is the amino acid sequence shown in SEQ ID NO: 18.

In specific embodiments of the present disclosure, the amino acid sequence of the Cas12 protein comprises or consists of an amino acid sequence that, as compared with SEQ ID NO: 18, has amino acid differences at any one of the following positions:
S211, Q216, N217, E218, K219, E220, K351, H352, N353, 1355, E359, A362, L363, A366, N365, L370, K401, V402, A403, E439, E463, D468, D276, E287, D270, E265, N224, D413, D417, A410, D428, E424, Q1005, N991, E999, L998, S995, D762, E761, N763, S843, S836, N833, A829, D768, Y988, E24, K76, Q80, Q282, L254, L240, E241, D302, N441, D393, G394, N395, S481, D157, E159, Q491, V490, H485, D903, D953, N904, V955, Q908, L932, S939, Q930, N870, E851, Q854, V850, N873, V872, D839, Q868, D800, E804, R19, R28, R32, K512, N527, W531, R553, K581, K589, 1590, R605, K611, R612, R615, Y777, E877, R931, H271, 1435, T436, F437, S438, D498, D639, D640 and T1006; the amino acid difference is a substitution of the residue at the position with any other residue.

In preferred embodiments of the present disclosure, the amino acid at the position is substituted with a positively charged amino acid, for example, R, H, or K; or the amino acid at the position is substituted with a nonpolar amino acid, for example, G, P, A, I, L, V, M, F, W or Y; or the amino acid at the position is substituted with a negatively charged amino acid, for example, D or E; or the amino acid at the position is substituted with a neutral amino acid, for example, N, C, Q, S or T.

In more preferred embodiments of the present disclosure, the amino acid at position Q216 or N217 is substituted with a positively charged amino acid or a nonpolar amino acid; or,
the amino acid at position S211, E218, K219, E220, K351, H352, N353, 1355, E359, A362, L363, A366, N365, L370, K401, V402, A403, E439, E463, D468, D276, E287, D270, E265, N224, D413, D417, A410, D428, E424, Q1005, N991, E999, L998, S995, D762, E761, N763, S843, S836, N833, A829, D768, Y988, K512, N527, W531, K581, K589, 1590, K611, Y777, E877, H271, D393, N395, 1435, T436, F437, S438, D498, D639, D640, V850, or T1006 is substituted with a positively charged amino acid; or,
the amino acid at position R19, R28, R32, R553, R605, R612, R615, or R931 is substituted with a positively charged amino acid, a nonpolar amino acid, a negatively charged amino acid, or a neutral amino acid.

In specific embodiments of the present disclosure, the gene-editing efficiency of the Cas12 protein is increased by at least 10% compared with the gene-editing efficiency of the Cas12 protein having the sequence of SEQ ID NO: 18.

In specific embodiments of the present disclosure, the gene-editing efficiency of the Cas12 protein is increased by at least 20%, at least 30%, at least 40%, at least 50%, at least 60%, at least 70%, at least 80%, at least 90%, at least 100%, at least 110%, at least 120%, at least 150%, at least 180%, at least 200%, at least 210%, at least 220%, at least 230%, at least 240%, at least 250%, at least 260%, or at least 270% compared with the gene-editing efficiency of the Cas12 protein having the sequence of SEQ ID NO: 18.

As used herein, “the gene-editing efficiency of the Cas12 protein is increased compared with the gene-editing efficiency of the Cas12 protein having the amino acid sequence of SEQ ID NO: 18” can refer to, for one particular guide sequence (without requiring two, three, or more guide sequences, and without requiring all guide sequences), the editing efficiency achieved by a combination of the Cas12 protein and a gRNA comprising that guide sequence being higher than the editing efficiency achieved by a combination of a Cas12 protein whose amino acid sequence comprises or is SEQ ID NO: 18 and a gRNA comprising that guide sequence.

In specific embodiments of the present disclosure, the amino acid sequence of the Cas12 protein comprises or consists of an amino acid sequence that, as compared with SEQ ID NO: 18, has any one of the following amino acid difference:
the amino acid at position Q216 or N217 is substituted with R, F, or W;
the amino acid at position S211, E218, K219, E220, K351, H352, N353, 1355, E359, A362, L363, A366, N365, L370, K401, V402, A403, E439, E463, D468, D276, E287, D270, E265, N224, D413, D417, A410, D428, E424, Q1005, N991, E999, L998, S995, D762, E761, N763, S843, S836, N833, A829, D768, Y988, K512, N527, W531, K581, K589, 1590, K611, Y777, E877, H271, D393, N395, 1435, T436, F437, S438, D498, D639, D640, V850, or T1006 is substituted with R; and,
the amino acid at position R19, R28, R32, R553, R605, R612, R615, or R931 is substituted with K, A, Q, or E.

In another aspect, the present disclosure provides a Cas12 protein mutant, wherein the amino acid sequence of the Cas12 protein mutant comprises or is an amino acid sequence having at least 70% sequence identity to SEQ ID NO: 40, and,
as compared with SEQ ID NO: 40, the Cas12 protein mutant has an amino acid difference at one or more positions selected from the following: S211, Q216, N217, E218, K219, E220, K351, H352, N353, 1355, E359, A362, L363, A366, N365, L370, K401, V402, A403, E439, E463, D468, D276, D287, D270, E265, N224, D413, D417, A410, D428, E424, Q1005, N991, E999, L998, S995, D762, E761, N763, S843, S836, N833, A829, D768, Y988, E24, K76, Q80, Q282, L254, L240, E241, D302, N441, D393, G394, N395, S481, D157, E159, Q491, V490, H485, D903, D953, N904, V955, Q908, L932, S939, Q930, N870, E851, Q854, V850, N873, V872, D839, Q868, D800, E804, H271, T435, T436, F437, S438, D498, D639, D640 or T1006; and the amino acid difference is a substitution of the residue at the position with any other residue.

In preferred embodiments of the present disclosure, the amino acid at the position is substituted with a positively charged amino acid, for example, R, H, or K; or the amino acid at the position is substituted with a nonpolar amino acid, for example, G, P, A, I, L, V, M, F, W or Y; or the amino acid at the position is substituted with a negatively charged amino acid, for example, D or E; or the amino acid at the position is substituted with a neutral amino acid, for example, N, C, Q, S or T.

In more preferred embodiments of the present disclosure, the amino acid at position S216 or N217 is substituted with a positively charged amino acid or a nonpolar amino acid; or the amino acid at position S211, E218, K219, E220, K351, H352, N353, 1355, E359, A362, L363, A366, N365, L370, K401, V402, A403, E439, E463, D468, D276, D287, D270, E265, N224, D413, D417, A410, D428, E424, Q1005, N991, E999, L998, S995, D762, E761, N763, S843, S836, N833, A829, D768, Y988, H271, D393, N395, T435, T436, F437, S438, D498, D639, D640, V850 or T1006 is substituted with a positively charged amino acid.
or, as compared with SEQ ID NO: 40, the amino acid at position R19, R28, R32, R553, R605, R612, R615 or R931 is substituted with K, A, Q, or E.
or, as compared with SEQ ID NO: 40, the amino acid at position K512, N527, W531, K581, K589, 1590, K611, Y777 or E877 is substituted with a positively charged amino acid, for example, R, H, or K; preferably R.

In specific embodiments of the present disclosure, the at least 70% sequence identity is at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity.

In specific embodiments of the present disclosure, the Cas12 protein mutant retains the function of the protein shown in SEQ ID NO: 40.

In specific embodiments of the present disclosure, retaining the function of the protein shown in SEQ ID NO: 40 refers to retaining the ability to bind to a target nucleic acid that is complementary to the guide sequence of a guide polynucleotide and cleave the target nucleic acid, and/or retaining the ability to process an RNA transcript of the guide sequence into a guide polynucleotide molecule.

In specific embodiments of the present disclosure, retaining the function of the protein shown in SEQ ID NO: 40 refers to retaining the ability to form a complex with a guide polynucleotide, retaining the ability to bind to a target nucleic acid that is complementary to the guide sequence of the guide polynucleotide, retaining the ability to cleave the target nucleic acid when guided by the guide polynucleotide, and/or retaining the ability to process an RNA transcript of the guide sequence into a guide polynucleotide molecule.

In specific embodiments of the present disclosure, retaining the function of the protein shown in SEQ ID NO: 40 refers to retaining the ability to form a complex with a guide polynucleotide.

In specific embodiments of the present disclosure, retaining the function of the protein shown in SEQ ID NO: 40 refers to retaining the ability to bind to a target nucleic acid that is complementary to the guide sequence of the guide polynucleotide.

In specific embodiments of the present disclosure, retaining the function of the protein shown in SEQ ID NO: 40 refers to retaining the ability to cleave a target nucleic acid when guided by a guide polynucleotide.

In specific embodiments of the present disclosure, retaining the function of the protein shown in SEQ ID NO: 40 refers to retaining the ability to process an RNA transcript of the guide sequence into a guide polynucleotide molecule.

In specific embodiments of the present disclosure, the Cas12 protein mutant can form a complex with a guide polynucleotide.

In specific embodiments of the present disclosure, the Cas12 protein mutant can form a complex with a guide polynucleotide. In specific embodiments of the present disclosure, the Cas12 protein mutant can bind to a target nucleic acid in a sequence-specific manner with a guide polynucleotide.

In specific embodiments of the present disclosure, the Cas12 protein mutant can bind to and cleave a target nucleic acid in a sequence-specific manner with a guide polynucleotide. In specific embodiments of the present disclosure, the Cas12 protein mutant can form a complex with a guide polynucleotide, and the complex can bind to and cleave the target nucleic acid in a sequence-specific manner.

In specific embodiments of the present disclosure, the gene-editing efficiency of the Cas12 protein mutant is increased by at least 10% compared with the gene-editing efficiency of the Cas12 protein having the sequence of SEQ ID NO: 40.

In specific embodiments of the present disclosure, the Cas12 mutant recognizes a 5′-TTN PAM sequence, such as TTA, TTT, TTC or TTG; and wherein N is A, T, C or G.

In specific embodiments of the present disclosure, the gene-editing efficiency of the Cas12 protein mutant is increased by at least 10% compared with the gene-editing efficiency of the Cas12 protein having the sequence of SEQ ID NO: 40; and/or the Cas12 protein mutant recognizes a 5′-TTN PAM sequence, wherein N is A, T, C, or G.

In preferred embodiments of the present disclosure, the gene-editing efficiency of the Cas12 protein mutant is increased by at least 20%, at least 30%, at least 40%, at least 50%, at least 60%, at least 70%, at least 80%, at least 90%, at least 100%, at least 110%, at least 120%, at least 150%, at least 180%, at least 200%, at least 210%, at least 220%, at least 230%, at least 240%, at least 250%, at least 260%, or at least 270% compared with the gene-editing efficiency of the Cas12 protein having the amino acid sequence shown in SEQ ID NO: 40.

In the present disclosure, “the gene-editing efficiency of the Cas12 protein is increased compared with the gene-editing efficiency of the Cas12 protein having the amino acid sequence of SEQ ID NO: 40” can refer to, for a particular guide sequence (without limiting to two, three, or more guide sequences, and without limiting to all guide sequences), the editing efficiency of the Cas12 protein in combination with a gRNA comprising the guide sequence being higher than the editing efficiency of the Cas12 protein comprising or consisting of SEQ ID NO: 40 in combination with a gRNA comprising the same guide sequence.

In specific embodiments of the present disclosure, the amino acid sequence of the Cas12 protein mutant comprises or consists of an amino acid sequence that, as compared with SEQ ID NO: 40, has an amino acid difference selected from any one of the following:
the amino acid at position Q216 or N217 is substituted with R, F, or W;
the amino acid at position S211, E218, K219, E220, K351, H352, N353, 1355, E359, A362, L363, A366, N365, L370, K401, V402, A403, E439, E463, D468, D276, D287, D270, E265, N224, D413, D417, A410, D428, E424, Q1005, N991, E999, L998, S995, D762, E761, N763, S843, S836, N833, A829, D768, Y988, H271, D393, N395, T435, T436, F437, S438, D498, D639, D640, V850, or T1006 is substituted with R.

In another aspect, the present disclosure provides a guide polynucleotide, comprising (i) a direct repeat sequence having the sequence of SEQ ID NO: 26, and (ii) a guide sequence engineered to hybridize to a target nucleic acid; wherein the direct repeat sequence is linked to the guide sequence; and wherein the guide polynucleotide can form a complex with a Cas12 protein and guide the complex to bind to the target nucleic acid in a sequence-specific manner.

In preferred embodiments of the present disclosure, the Cas12 protein is the Cas12 protein as described in the present disclosure or the Cas12 protein mutant as described in the present disclosure.

In specific embodiments of the present disclosure, the guide sequence comprises 15-35 nucleotides, and/or the guide sequence hybridizes to the target nucleic acid, wherein the guide sequence and the target nucleic acid are 90%-100% complementary, preferably with no more than one nucleotide mismatch; for example, the nucleotide sequence of the guide sequence is as shown in any one of SEQ ID NOs: 27-28.

In specific embodiments of the present disclosure, the guide sequence is located at the 3′ end of the direct repeat sequence; for example, the nucleotide sequence of the guide polynucleotide is optionally selected from any one of SEQ ID NOs: 24-25.

In another aspect, the present disclosure provides a Cas12 protein, wherein the Cas12 protein comprises a Cas12 active fragment, and the Cas12 active fragment comprises one or more domains selected from: the Helical-I1 domain, PI domain, Helical-II domain, Ruvc-I domain, Helical-III domain, and Nuc domain of the Cas12 protein as described in the present disclosure;
or the Cas12 active fragment comprises one or more domains selected from: the WED-I domain, Helical-I1 domain, PI domain, Helical-12 domain, Helical-II domain, WED-II domain, Helical-III domain, BH domain, Ruvc-II domain, and Nuc domain of the Cas12 protein as described in the present disclosure; and the Cas12 active fragment comprises the amino acid difference as defined in the Cas12 protein as described in the present disclosure.

In preferred embodiments of the present disclosure, the Cas12 active fragment comprises the PI domain and comprises one or more domains selected from: the Helical-I1 domain, Helical-12 domain, Helical-II domain, Helical-III domain, and BH domain;
or the Cas12 active fragment comprises the WED-I domain, WED-II domain, Ruvc-I domain, Ruvc-II domain, and Nuc domain, and further comprises the Ruvc-III domain of the Cas12 protein as described in the present disclosure.

In more preferred embodiments of the present disclosure, the Cas12 active fragment comprises the PI domain, Helical-I1 domain, Helical-I2 domain, Helical-II domain, Helical-III domain, and BH domain.

In specific embodiments of the present disclosure, the Cas12 active fragment comprises one or more domains selected from: the WED-I domain, Helical-I1 domain, PI domain, Helical-12 domain, Helical-II domain, WED-II domain, Helical-III domain, BH domain, Ruvc-II domain, and Nuc domain of the Cas12 protein mutant as described in the present disclosure; and the Cas12 active fragment comprises the amino acid difference as defined in the Cas12 protein mutant as described in the present disclosure.

The delineation of the domains of the Cas12 protein can be determined by sequence alignment with the protein shown in SEQ ID NO: 18 or 40.

In another aspect, the present disclosure provides a Cas12 inactivated variant, wherein the Cas12 inactivated variant is a nuclease-activity-inactivated variant of the Cas12 protein as described in the present disclosure or the Cas12 protein mutant as described in the present disclosure.

In specific embodiments of the present disclosure, the Cas12 inactivated variant is a completely nuclease-inactivated variant, i.e., a dead Cas12 inactivated variant (dCas12). The dCas12 can bind to a target nucleic acid only when mediated by a guide polynucleotide, but does not have, or has almost no, the function of cleaving the target nucleic acid. For example, the target nucleic acid cleavage efficiency of the dCas12 is ≤10%, ≤5%, ≤4%, ≤3%, ≤2%, or ≤1% of the target nucleic acid cleavage efficiency of the Cas12 protein or Cas12 protein mutant before introduction of the inactivating mutation.

In specific embodiments of the present disclosure, the Cas12 inactivated variant is a partially nuclease-inactivated variant. Further, the partially nuclease-inactivated variant is a Cas12 nickase (nickase Cas12, nCas12), which, when mediated by a guide polynucleotide, binds to a target nucleic acid and then cleaves one strand of a double-stranded target nucleic acid, but does not cleave the other strand.

In preferred embodiments of the present disclosure, the Cas12 inactivated variant has an inactivated Ruvc domain of the Cas12 protein or the Cas12 protein mutant.

In preferred embodiments of the present disclosure, the Cas12 inactivated variant has an inactivated Ruvc-I, Ruvc-II, or Ruvc-III domain of the Cas12 protein or the Cas12 protein mutant.

In preferred embodiments of the present disclosure, the Cas12 inactivated variant is obtained by introducing an inactivating mutation into the Ruvc-I, Ruvc-II, or Ruvc-III domain of the Cas12 protein or the Cas12 protein mutant.

In another aspect, the present disclosure provides a Cas12 fusion protein or conjugate, comprising the following elements: (1) a Cas12 functional domain, which comprises the Cas12 protein as described in the present disclosure, the Cas12 protein mutant as described in the present disclosure, or the Cas12 inactivated variant as described in the present disclosure; and (2) a homologous or heterologous functional domain.

In specific embodiments of the present disclosure, the homologous or heterologous functional domain is optionally selected from one or more of the following: a subcellular localization signal, a DNA-binding domain, a protease domain, a transcriptional activation domain, a transcriptional repression domain, a nuclease domain, a deaminase domain, a uracil DNA glycosylase domain (UDG), a uracil DNA glycosylase inhibitor domain (UGI), a methyltransferase, a demethylase, a transcription release factor, a histone acetyltransferase domain, a histone deacetylase domain, a DNA ligase, an epitope tag, and a reporter domain.

In preferred embodiments of the present disclosure, the nuclease domain comprises a polypeptide having ssDNA cleavage activity and/or a polypeptide having dsDNA cleavage activity.

In specific embodiments of the present disclosure, the Cas12 functional domain is directly or indirectly linked to the homologous or heterologous functional domain.

In preferred embodiments of the present disclosure, the direct linkage is a covalent linkage, and the indirect linkage is a linkage via an amino acid linker or a non-amino acid linker.

In more preferred embodiments of the present disclosure, the homologous or heterologous functional domain is fused or conjugated to the N-terminus, the C-terminus, or an internal region of the Cas12 functional domain.

In the present disclosure, a fusion protein refers to a protein in which element (1) and element (2) are linked via a peptide segment or are directly linked; and a conjugate refers to a product in which element (1) and element (2) are linked via a chemical bond that is not a peptide bond.

In another aspect, the present disclosure provides an isolated nucleic acid encoding the Cas12 protein as described in the present disclosure, the Cas12 protein mutant as described in the present disclosure, the Cas12 inactivated variant as described in the present disclosure, or the Cas12 fusion protein or conjugate as described in the present disclosure.

In preferred embodiments of the present disclosure, the nucleic acid is codon optimized for expression in cells.

In more preferred embodiments of the present disclosure, the nucleic acid is codon optimized for expression in a eukaryote, a mammal such as a human or a non-human mammal, a plant, an insect, a bird, a reptile, a rodent (e.g., a mouse or a rat), a fish, a worm/nematode, or yeast.

In another aspect, the present disclosure provides a CRISPR-Cas12 system, comprising:
a. a Cas12 functional domain, the Cas12 fusion protein or conjugate as described in the present disclosure, or the nucleic acid as described in the present disclosure, wherein the Cas12 functional domain comprises the Cas12 protein as described in the present disclosure, the Cas12 protein mutant as described in the present disclosure, or the Cas12 inactivated variant as described in the present disclosure; and
b. a guide polynucleotide, or a polynucleotide sequence encoding the guide polynucleotide;
wherein the Cas12 functional domain or the Cas12 fusion protein or conjugate forms a complex with the guide polynucleotide; and the guide polynucleotide comprises a guide sequence that is engineered to guide the complex to bind to a target nucleic acid in a sequence-specific manner.

In specific embodiments of the present disclosure, the guide polynucleotide comprises a direct repeat sequence linked to the guide sequence.

In specific embodiments of the present disclosure, the nucleotide sequence of the direct repeat sequence is as shown in SEQ ID NO: 26 or SEQ ID NO: 41.

In specific embodiments of the present disclosure, the guide polynucleotide comprises a direct repeat sequence linked to the guide sequence. Further, in some specific embodiments, the direct repeat sequence has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, or at least 98% sequence identity to the sequence shown in SEQ ID NO: 9. Further, in some specific embodiments, the direct repeat sequence comprises or consists of the sequence shown in SEQ ID NO: 26 or SEQ ID NO: 41.

In specific embodiments of the present disclosure, the guide sequence comprises 15-35 nucleotides, and/or the guide sequence hybridizes to the target nucleic acid, wherein the guide sequence and the target nucleic acid are 90%-100% complementary, preferably with no more than one nucleotide mismatch.

In specific embodiments of the present disclosure, the guide sequence is located at the 5′ end or the 3′ end of the direct repeat sequence.

In specific embodiments of the present disclosure, the guide sequence is located at the 5′ end of the direct repeat sequence.

In specific embodiments of the present disclosure, the guide sequence is located at the 3′ end of the direct repeat sequence.

In preferred embodiments of the present disclosure, the guide polynucleotide is the guide polynucleotide as described in the present disclosure.

In specific embodiments of the present disclosure, the target nucleic acid is DNA or RNA, preferably dsDNA or ssDNA.

In preferred embodiments of the present disclosure, the DNA is eukaryotic DNA; preferably, the eukaryotic DNA is non-human mammal DNA, non-human primate DNA, human DNA, plant DNA, insect DNA, bird DNA, reptile DNA, rodent DNA (e.g., mouse DNA or rat DNA), fish DNA, worm/nematode DNA, or yeast DNA.

In specific embodiments of the present disclosure, the target nucleic acid is a disease- or disorder-related gene or a signaling pathway-related gene, or the target nucleic acid is a reporter gene; for example, the disease or disorder is a hematologic disease or disorder, an ophthalmic disease or disorder, a nervous system disease or disorder, a respiratory system disease or disorder, a liver disease or disorder, a metabolic disease or disorder, cancer, or an infectious disease.

In another aspect, the present disclosure provides a vector system, comprising one or more recombinant vectors, wherein the recombinant vectors comprise the isolated nucleic acid as described in the present disclosure or the CRISPR-Cas12 system as described in the present disclosure.

In specific embodiments of the present disclosure, the recombinant vector further comprises a regulatory sequence.

In specific embodiments of the present disclosure, the vector system comprises one or more recombinant vectors, wherein the recombinant vectors comprise a polynucleotide sequence encoding the Cas12 protein as described in the present disclosure, the Cas12 protein mutant as described in the present disclosure, the Cas12 inactivated variant as described in the present disclosure, or the Cas12 fusion protein or conjugate as described in the present disclosure, and a polynucleotide sequence encoding the guide polynucleotide.

In specific embodiments of the present disclosure, the polynucleotide sequence encoding the Cas12 protein, the Cas12 protein mutant, the Cas12 inactivated variant, or the Cas12 fusion protein or conjugate is operably linked to regulatory sequence 1.

In specific embodiments of the present disclosure, the polynucleotide sequence encoding the guide polynucleotide is operably linked to regulatory sequence 2.

Further, in specific embodiments of the present disclosure, regulatory sequence 1 and regulatory sequence 2 are the same or different.

In preferred embodiments of the present disclosure, the regulatory sequence is optionally selected from one or more of the following: a promoter, an enhancer, an internal ribosome entry site, and a transcription termination signal; the promoter is, for example, a constitutive promoter, an inducible promoter, a ubiquitous promoter, or a tissue-specific promoter; and/or the transcription termination signal is, for example, a polyadenylation signal or a poly-U sequence.

In specific embodiments of the present disclosure, the backbone of the recombinant vector is an adeno-associated virus vector, a lentiviral vector, a ribonucleoprotein complex, or a virus-like particle.

In preferred embodiments of the present disclosure:
when the backbone is an adeno-associated virus vector, the adeno-associated virus vector is a recombinant adeno-associated virus vector of serotype AAV1, AAV2, AAV4, AAV5, AAV6, AAV7, AAVrh74, AAV8, AAV9, AAV10, AAV11, AAV12, or AAV13;
when the backbone is a lentiviral vector, the lentiviral vector is pseudotyped with an envelope protein; in specific embodiments of the present disclosure, the isolated nucleic acid is linked to an aptamer sequence;
when the backbone is a virus-like particle, the isolated nucleic acid is linked to a gene encoding a gag protein.

In another aspect, the present disclosure provides a delivery system, comprising: (1) a delivery tool, and (2) the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, or the vector system as described in the present disclosure.

In preferred embodiments of the present disclosure, the delivery tool is a virus, a lipid nanoparticle, a nanoparticle, a liposome, an exosome, a microbubble, or a gene gun.

In more preferred embodiments of the present disclosure, the delivery tool is a lipid nanoparticle, and the lipid nanoparticle comprises the guide polynucleotide and an mRNA encoding the Cas12 protein, the Cas12 inactivated variant, the Cas12 protein mutant, or the Cas12 fusion protein or conjugate.

In another aspect, the present disclosure provides a cell, wherein the cell comprises the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, or the vector system as described in the present disclosure.

In preferred embodiments of the present disclosure, the cell is a eukaryotic cell.

In more preferred embodiments of the present disclosure, the eukaryotic cell is a mammalian cell.

In another aspect, the present disclosure provides a pharmaceutical composition, wherein the pharmaceutical composition comprises the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, or the cell as described in the present disclosure.

In specific embodiments of the present disclosure, the pharmaceutical composition comprises a pharmaceutically acceptable excipient.

In another aspect, the present disclosure provides a kit, wherein the kit comprises the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, or the cell as described in the present disclosure.

In preferred embodiments of the present disclosure, the kit further comprises a cleavage buffer (Cut Buffer). The Cut Buffer can be any buffer known in the art that is suitable for the Cas12 protein to cleave a target nucleic acid.

The Cut Buffer preferably comprises Tris-HCl, KCl, MgCl2, DTT, glycerol, and ATP.

In more preferred embodiments of the present disclosure, the Cut Buffer meets one or more of the following conditions:
Tris-HCl has a pH of 7.0-8.0; Tris-HCl concentration is 180-220 mM; KCl concentration is 480-520 mM; MgCl2 concentration is 45-55 mM; DTT concentration is 4.5-5.5 mM; glycerol is 8%-12% (v/v); and ATP concentration is 0.8-1.2 mM.

The Cut Buffer is a 10× Cut Buffer and is present in the reaction system at one tenth of its concentration.

In another aspect, the present disclosure provides use of the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure in the manufacture of a reagent or medicament for diagnosing, treating, and/or preventing a disease or disorder related to a target nucleic acid.

In specific embodiments of the present disclosure, the disease or disorder is a hematologic disease or disorder, an ophthalmic disease or disorder, a nervous system disease or disorder, a respiratory system disease or disorder, a liver disease or disorder, a metabolic disease or disorder, cancer, or an infectious disease; and/or the reagent or medicament is used to: cleave one or more target nucleic acid molecules or introduce nicks into one or more target nucleic acid molecules; activate or upregulate expression of one or more target nucleic acid molecules; activate or inhibit transcription of one or more target nucleic acid molecules; inactivate one or more target nucleic acid molecules; visualize, label, or detect one or more target nucleic acid molecules; bind to one or more target nucleic acid molecules; transport one or more target nucleic acid molecules; and mask one or more target nucleic acid molecules.

In another aspect, the present disclosure provides a method for detecting, binding, or cleaving a target nucleic acid, comprising contacting the target nucleic acid with the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure.

In preferred embodiments of the present disclosure, the method is a method for non-diagnostic and/or non-therapeutic purposes; and/or the Cas12 fusion protein or conjugate comprises a detectable label, for example, a label detectable by fluorescence, DNA blotting, or fluorescence in situ hybridization (FISH).

In more preferred embodiments of the present disclosure, when the method is for cleaving a target nucleic acid, the method further comprises performing a cleavage reaction using a cleavage buffer (Cut Buffer). The Cut Buffer can be any buffer known in the art that is suitable for the Cas12 protein to cleave a target nucleic acid.

The Cut Buffer preferably comprises Tris-HCl, KCl, MgCl2, DTT, glycerol, and ATP.

In further more preferred embodiments of the present disclosure, the Cut Buffer meets one or more of the following conditions:
Tris-HCl has a pH of 7.0-8.0; Tris-HCl concentration is 180-220 mM; KCl concentration is 480-520 mM; MgCl2 concentration is 45-55 mM; DTT concentration is 4.5-5.5 mM; glycerol is 8%-12% (v/v); and ATP concentration is 0.8-1.2 mM.

The Cut Buffer is a 10× Cut Buffer and is present in the reaction system at one tenth of its concentration.

In another aspect, the present disclosure provides a method for altering a cell state, comprising contacting a cell with the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure, thereby altering the cell state.

In preferred embodiments of the present disclosure, the method results in one or more of the following: (i) inducing cellular senescence in vitro or in vivo; (ii) cell cycle arrest in vitro or in vivo; (iii) promoting and/or inhibiting cell growth in vitro or in vivo; (iv) inducing unresponsiveness in vitro or in vivo; (v) inducing apoptosis in vitro or in vivo; and (vi) inducing necrosis in vitro or in vivo.

In more preferred embodiments of the present disclosure, the method is a method for non-diagnostic and/or non-therapeutic purposes.

In another aspect, the present disclosure provides a method for diagnosing, treating, or preventing a disease or disorder related to a target nucleic acid, comprising administering, to a sample from a subject in need thereof or to a subject in need thereof, the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure.

In specific embodiments of the present disclosure, the disease or disorder is a hematologic disease or disorder, an ophthalmic disease or disorder, a nervous system disease or disorder, a respiratory system disease or disorder, a liver disease or disorder, a metabolic disease or disorder, cancer, or an infectious disease.

In another aspect, the present disclosure provides the Cas12 protein as described in the present disclosure, the Cas12 protein mutant as described in the present disclosure, the guide polynucleotide as described in the present disclosure, the Cas12 inactivated variant as described in the present disclosure, the Cas12 fusion protein or conjugate as described in the present disclosure, the nucleic acid as described in the present disclosure, the CRISPR-Cas12 system as described in the present disclosure, the vector system as described in the present disclosure, the delivery system as described in the present disclosure, the cell as described in the present disclosure, the pharmaceutical composition as described in the present disclosure, or the kit as described in the present disclosure for use in diagnosing, treating, or preventing a disease or disorder related to a target nucleic acid.

In specific embodiments of the present disclosure, the disease or disorder is a hematologic disease or disorder, an ophthalmic disease or disorder, a nervous system disease or disorder, a respiratory system disease or disorder, a liver disease or disorder, a metabolic disease or disorder, cancer, or an infectious disease.

The present disclosure provides, in one aspect, a Cas12 protein, wherein the amino acid sequence of the Cas12 protein comprises or consists of an amino acid sequence having at least 50% sequence identity to SEQ ID NO: 1, and wherein the amino acid sequence of the Cas12 protein comprises or consists of an amino acid sequence that, as compared with SEQ ID NO: 1, has amino acid differences at positions N260, N295, and G705, and further has an amino acid difference at one, two, or more positions selected from the following:
D166, V167, N168, G169, W170, S174, E179, K181, K182, E183, E184, Q294, E328, K370, N372, E376, E397, E462, V463, N621, D851, S853, A934, W938, N941, K942, K943, N945, N197, E788, K228, K231, E326, L329, K353, P362, G366, N368, N369, Y371, A392, K395, D396, E399, E400, K401, G402, I403, H405, K408, E434, S433, K441, C448, G455, K502, T505, V842, K580R, T623, K774, S775, T850, K856, K926, Q929, N930, S940, S944, K580, S779, H511, N523, P524, P1032, P579, P984, L767, H995, P557, G232, and L662;
the amino acid difference is a substitution of the residue at the position with any other residue.

In specific embodiments of the present disclosure, the amino acid sequence of the Cas12 protein comprises or consists of an amino acid sequence having at least 80% sequence identity to SEQ ID NO: 1.

In specific embodiments of the present disclosure, the amino acid sequence of the Cas12 protein comprises or consists of an amino acid sequence having at least 85% sequence identity to SEQ ID NO: 1.

In specific embodiments of the present disclosure, the amino acid sequence of the Cas12 protein comprises or consists of an amino acid sequence having at least 90%, at least 95%, at least 96%, at least 97%, at least 98%, or at least 99% sequence identity to SEQ ID NO: 1.

Optionally, the Cas12 protein can recognize a 5′-TTN PAM sequence.

In specific embodiments of the present disclosure, the gene-editing efficiency of the Cas12 protein is increased by at least 10% compared with the gene-editing efficiency of the Cas12 protein having the sequence of SEQ ID NO: 1.

In specific embodiments of the present disclosure, the gene-editing efficiency of the Cas12 protein is increased by at least 20%, at least 30%, at least 40%, at least 50%, at least 60%, at least 70%, at least 80%, at least 90%, at least 100%, at least 110%, at least 120%, at least 150%, at least 180%, at least 200%, at least 210%, at least 220%, at least 230%, at least 240%, at least 250%, at least 260%, or at least 270% compared with the gene-editing efficiency of the Cas12 protein having the sequence of SEQ ID NO: 1.

In specific embodiments of the present disclosure, the gene-editing efficiency is the editing efficiency targeting the reporter system in Example 1 of the present disclosure. In specific embodiments of the present disclosure, the gene-editing efficiency is the editing efficiency of the Cas12 protein in combination with a gRNA as shown in any one of SEQ ID NOs: 10-12 in human cells. In specific embodiments of the present disclosure, the gene-editing efficiency is the editing efficiency of the Cas12 protein in combination with a gRNA as shown in any one of SEQ ID NOs: 10-12 in 293T cells. In specific embodiments of the present disclosure, the gene-editing efficiency is the editing efficiency of the Cas12 protein in combination with a gRNA comprising a guide sequence as shown in any one of SEQ ID NOs: 14-16 in human cells. In specific embodiments of the present disclosure, the gene-editing efficiency is the editing efficiency of the Cas12 protein in combination with a gRNA comprising a guide sequence as shown in any one of SEQ ID NOs: 14-16 in 293T cells.

In specific embodiments of the present disclosure, the gene-editing efficiency is the efficiency of introducing an indel. In specific embodiments of the present disclosure, the gene-editing efficiency is the single-base editing efficiency of the Cas12 protein, or the fusion protein or conjugate. In specific embodiments of the present disclosure, the gene-editing efficiency is the efficiency of transcription activation or transcription repression caused by the Cas12 protein, or the fusion protein or conjugate. The gene-editing efficiency can be tested by conventional methods in the art.

In specific embodiments of the present disclosure, the amino acid sequence of the Cas12 protein comprises or consists of an amino acid sequence that, as compared with SEQ ID NO: 1, comprises amino acid differences at positions N260, N295, and G705, and has an amino acid difference at position:
D166, V167, N168, G169, W170, S174, E179, K181, K182, E183, E184, Q294, E328, K370, N372, E376, E397, E462, V463, N621, D851, S853, A934, W938, N941, K942, K943, N945, N197, E788, K228, K231, E326, L329, K353, P362, G366, N368, N369, Y371, A392, K395, D396, E399, E400, K401, G402, I403, H405, K408, E434, S433, K441, C448, G455, K502, T505, V842, K580R, T623, K774, S775, T850, K856, K926, Q929, N930, S940, S944, K580, S779, H511, N523, P524, P1032, P579, P984, L767, H995, P557, G232, or L662; or,
E788 and N197; or,
T505 and V842; or,
H511 and N523; or,
N523 and P524.

In specific embodiments of the present disclosure, the amino acid difference is a substitution of the amino acids at position N260, N295, G705, E179, K181, K182, E183, E184, E328, K370, N372, E376, E397, E462, V463, D851, S853, A934, W938, N941, K942, K943, N945, E788, K228, K231, E326, L329, K353, P362, G366, N368, N369, Y371, A392, K395, D396, E399, E400, K401, G402, I403, H405, K408, E434, S433, K441, G455, K502, T505, K580, T623, K774, S775, S779, T850, K856, K926, Q929, N930, S940, S944, N523, P524, P1032, P579, P984, P557, or N197 with a positively charged amino acid, such as R, H, or K; and/or,
the residues at positions G232 and N621 are substituted with negatively charged residues, such as D or E; and/or,
the residues at positions H511 and H995 are substituted with a neutral residue, such as N, C, Q, S, or T; and/or,
the residues at positions D166, N168, W170, S174, Q294, C448, V842, L767, and L662 are substituted with a nonpolar amino acid, such as G, P, A, I, L, V, M, F, W, or Y; and/or,
the residues at positions V167 and G169 are substituted with positively charged residues, such as R, H, or K; or are substituted with nonpolar residues, such as G, P, A, I, L, V, M, F, W, or Y.

In specific embodiments of the present disclosure, the amino acid sequence of the Cas12 protein comprises or consists of a sequence that, as compared with SEQ ID NO: 1, comprises amino acid differences of N260R, N295R, and G705R, and further comprises one, two, or more amino acid differences selected from the following:
D166W, D166F, V167W, V167F, V167R, N168W, N168F, G169W, G169F, G169R, W170F, S174W, S174F, E179R, K181R, K182R, E183R, E184R, Q294W, Q294F, E328R, K370R, N372R, E376R, E397R, E462R, V463R, N621D, D851R, S853R, A934R, W938R, N941R, K942R, K943R, N945R, N197K, E788R, K228R, K231R, L329R, K353R, P362R, G366R, N368R, N369R, Y371R, A392R, K395R, D396R, E399R, E400R, K401R, G402R, I403R, H405R, K408R, E434R, S433R, K441R, C448A, G455R, K502R, T505R, V842I, K580R, T623R, K774R, S775R, S779R, T850R, K856R, K926R, Q929R, N930R, S940R, S944R, H511N, N523H, P524H, P1032H, P579H, P984H, L767M, H995N, P557H, G232D, or L662M.

In specific embodiments of the present disclosure, the amino acid sequence of the Cas12 protein comprises or consists of a sequence that, as compared with SEQ ID NO: 1, comprises amino acid differences N260R, N295R, and G705R, and
further comprises the following amino acid difference: D166W, D166F, V167W, V167F, V167R, N168W, N168F, G169W, G169F, G169R, W170F, S174W, S174F, E179R, K181R, K182R, E183R, E184R, Q294W, Q294F, E328R, K370R, N372R, E376R, E397R, E462R, V463R, N621D, D851R, S853R, A934R, W938R, N941R, K942R, K943R, N945R, N197K, E788R, K228R, K231R, E326R, L329R, K353R, P362R, G366R, N368R, N369R, Y371R, A392R, K395R, D396R, E399R, E400R, K401R, G402R, I403R, H405R, K408R, E434R, S433R, K441R, C448A, G455R, K502R, K580R, T623R, K774R, S775R, S779R, T850R, K856R, K926R, Q929R, N930R, S940R, S944R, H511N, P524H, P1032H, P579H, P984H, L767M, H995N, P557H, G232D, or L662M; or, also comprises amino acid differences: E788R and N197K; or,
also comprises amino acid differences: T505R and V842I; or,
also comprises amino acid differences: H511N and N523H; or,
also comprises amino acid differences: N523H and P524H.

In specific embodiments of the present disclosure, the amino acid sequence of the Cas12 protein comprises or consists of a sequence that, as compared with SEQ ID NO: 1, has amino acid differences of:
N295R, N260R, G705R, and D166W;
N295R, N260R, G705R, and D166F;
N295R, N260R, G705R, and V167W;
N295R, N260R, G705R, and V167F;
N295R, N260R, G705R, and V167R;
N295R, N260R, G705R, and N168W;
N295R, N260R, G705R, and N168F;
N295R, N260R, G705R, and G169W;
N295R, N260R, G705R, and G169F;
N295R, N260R, G705R, and G169R;
N295R, N260R, G705R, and W170F;
N295R, N260R, G705R, and S174W;
N295R, N260R, G705R, and S174F;
N295R, N260R, G705R, and E179R;
N295R, N260R, G705R, and K181R;
N295R, N260R, G705R, and K182R;
N295R, N260R, G705R, and E183R;
N295R, N260R, G705R, and E184R;
N295R, N260R, G705R, and Q294W;
N295R, N260R, G705R, and Q294F;
N295R, N260R, G705R, and E328R;
N295R, N260R, G705R, and K370R;
N295R, N260R, G705R, and N372R;
N295R, N260R, G705R, and E376R;
N295R, N260R, G705R, and E397R;
N295R, N260R, G705R, and E462R;
N295R, N260R, G705R, and V463R;
N295R, N260R, G705R, and N621D;
N295R, N260R, G705R, and D851R;
N295R, N260R, G705R, and S853R;
N295R, N260R, G705R, and A934R;
N295R, N260R, G705R, and W938R;
N295R, N260R, G705R, and N941R;
N295R, N260R, G705R, and K942R;
N295R, N260R, G705R, and K943R;
N295R, N260R, G705R, and N945R;
N295R, N260R, G705R, and N197K;
N295R, N260R, G705R, and E788R;
N295R, N260R, G705R, E788R, and N197K;
N295R, N260R, G705R, and K181R;
N295R, N260R, G705R, and K228R;
N295R, N260R, G705R, and K231R;
N295R, N260R, G705R, and E326R;
N295R, N260R, G705R, and L329R;
N295R, N260R, G705R, and K353R;
N295R, N260R, G705R, and P362R;
N295R, N260R, G705R, and G366R;
N295R, N260R, G705R, and N368R;
N295R, N260R, G705R, and N369R;
N295R, N260R, G705R, and K370R;
N295R, N260R, G705R, and Y371R;
N295R, N260R, G705R, and N372R;
N295R, N260R, G705R, and A392R;
N295R, N260R, G705R, and K395R;
N295R, N260R, G705R, and D396R;
N295R, N260R, G705R, and E399R;
N295R, N260R, G705R, and E400R;
N295R, N260R, G705R, and K401R;
N295R, N260R, G705R, and G402R;
N295R, N260R, G705R, and I403R;
N295R, N260R, G705R, and H405R;
N295R, N260R, G705R, and K408R;
N295R, N260R, G705R, and E434R;
N295R, N260R, G705R, and S433R;
N295R, N260R, G705R, and K441R;
N295R, N260R, G705R, and C448A;
N295R, N260R, G705R, and G455R;
N295R, N260R, G705R, and K502R;
N295R, N260R, G705R, T505R, and V842I;
N295R, N260R, G705R, and K580R;
N295R, N260R, G705R, and T623R;
N295R, N260R, G705R, and K774R;
N295R, N260R, G705R, and S775R;
N295R, N260R, G705R, and S779R;
N295R, N260R, G705R, and T850R;
N295R, N260R, G705R, and K856R;
N295R, N260R, G705R, and K926R;
N295R, N260R, G705R, and Q929R;
N295R, N260R, G705R and N930R;
N295R, N260R, G705R and S940R;
N295R, N260R, G705R and S944R;
N295R, N260R, G705R and E326R;
N295R, N260R, G705R and Y371R;
N295R, N260R, G705R and E434R;
N295R, N260R, G705R and K580R;
N295R, N260R, G705R and K774R;
N295R, N260R, G705R and S779R;
N295R, N260R, G705R and H511N;
N295R, N260R, G705R, H511N and N523H;
N295R, N260R, G705R and P524H;
N295R, N260R, G705R, N523H and P524H;
N295R, N260R, G705R and P1032H;
N295R, N260R, G705R and P579H;
N295R, N260R, G705R and P984H;
N295R, N260R, G705R and L767M;
N295R, N260R, G705R and H995N;
N295R, N260R, G705R and P557H;
N295R, N260R, G705R and G232D; or,
N295R, N260R, G705R and L662M.

Optionally, the Cas12 protein can recognize a 5′-TTN PAM sequence, wherein the N is A, T, C, or G.

The present disclosure provides, in another aspect, a fusion protein or conjugate, wherein the fusion protein or conjugate comprises a Cas12 protein as described in the present disclosure, or a functional fragment thereof, fused to a homologous or heterologous functional domain.

In some embodiments, fusion of the Cas12 protein does not alter the original functions of the Cas12 protein, including but not limited to binding to and cleaving a target nucleic acid.

In some embodiments of the present disclosure, the homologous or heterologous functional domain is optionally selected from one or more of: a subcellular localization signal, a DNA-binding domain, a protein targeting moiety, a transcriptional activation domain, a transcriptional repression domain, a nuclease, a base editing domain such as a deaminase domain, a methyltransferase, a demethylase, a transcription release factor, a histone deacetylase, a polypeptide having ssDNA cleavage activity, a polypeptide having dsDNA cleavage activity, a DNA ligase, an epitope tag, a reporter protein, and a detection label.

In some embodiments of the present disclosure, the Cas12 protein is covalently linked to the homologous or heterologous functional domain.

In some embodiments of the present disclosure, the Cas12 protein is directly linked to the homologous or heterologous functional domain, or is covalently linked via an amino acid linker or a non-amino acid linker.

In some embodiments of the present disclosure, the homologous or heterologous functional domain is fused or conjugated to the Cas12 protein at the N-terminus, the C-terminus, or internally.

Optionally, the fusion protein or conjugate can recognize a 5′-TTN PAM sequence, wherein N is A, T, C, or G.

The present disclosure provides, in another aspect, an isolated nucleic acid encoding a Cas12 protein as described in the present disclosure, or a fusion protein or conjugate as described in the present disclosure.

In some embodiments of the present disclosure, the nucleic acid is codon-optimized for expression in a cell.

In some embodiments of the present disclosure, the nucleic acid is codon-optimized for expression in a eukaryote, a mammal (such as a human or a non-human mammal), a plant, an insect, a bird, a reptile, a rodent (e.g., a mouse or a rat), a fish, a worm/nematode, or yeast.

The present disclosure provides, in another aspect, a CRISPR-Cas12 system, wherein the CRISPR-Cas12 system comprises:
a. a Cas12 protein as described in the present disclosure, a fusion protein or conjugate as described in the present disclosure, or a nucleic acid as described in the present disclosure;
and
b. a guide polynucleotide, or a polynucleotide sequence encoding the guide polynucleotide;
wherein the Cas12 protein or the fusion protein or conjugate and the guide polynucleotide form a CRISPR complex; and wherein the guide polynucleotide comprises a guide sequence, and the guide sequence is engineered to guide the CRISPR complex to bind to a target nucleic acid in a sequence-specific manner.

In some embodiments of the present disclosure, the guide polynucleotide comprises a direct repeat sequence linked to the guide sequence; and the nucleotide sequence of the direct repeat sequence has at least 80% identity to SEQ ID NO: 17.

In some embodiments of the present disclosure, the nucleotide sequence of the direct repeat sequence is as set forth in SEQ ID NO: 17.

In some embodiments of the present disclosure, the target nucleic acid is DNA or RNA, preferably dsDNA or ssDNA.

In some embodiments of the present disclosure, the DNA is eukaryotic DNA; preferably, the eukaryotic DNA is non-human mammalian DNA, non-human primate DNA, human DNA, plant DNA, insect DNA, bird DNA, reptile DNA, rodent DNA (e.g., mouse DNA or rat DNA), fish DNA, worm/nematode DNA, or yeast DNA.

In some embodiments of the present disclosure, the target nucleic acid is a disease-associated gene or a gene associated with a signal transduction biochemical pathway; or the target nucleic acid is a reporter gene.

In some embodiments of the present disclosure, the disease-associated gene or the gene associated with the signal transduction biochemical pathway is the TTR (transthyretin), HBB (hemoglobin beta), or HBG (hemoglobin gamma-globin) gene; and the reporter gene is the GFP (green fluorescent protein) gene.

In some embodiments of the present disclosure, the guide sequence comprises 15-35 nucleotides; and/or the guide sequence hybridizes to the target nucleic acid, wherein the guide sequence and the target nucleic acid are 90%-100% complementary, preferably with no more than one nucleotide mismatch. In some embodiments of the present disclosure, the guide sequence is optionally selected from the sequences set forth in SEQ ID NOs: 14-16.

In some embodiments of the present disclosure, the guide sequence is located at the 3′ end of the direct repeat sequence.

The present disclosure provides, in another aspect, a vector system comprising one or more vectors, wherein the vector comprises an isolated nucleic acid as described in the present disclosure, or a CRISPR-Cas12 system as described in the present disclosure.

In some embodiments of the present disclosure, the vector further comprises a regulatory sequence.

In some embodiments of the present disclosure, the regulatory sequence comprises one or more selected from: a promoter, an enhancer, an internal ribosome entry site, and a transcription termination signal; the promoter is, for example, a constitutive promoter, an inducible promoter, a ubiquitous promoter, or a tissue-specific promoter; and/or the transcription termination signal is, for example, a polyadenylation signal or a poly-U sequence.

In some embodiments of the present disclosure, the regulatory sequence is operably linked to the vector.

In some embodiments of the present disclosure, the backbone of the vector is pcDNA3.1.

In some embodiments of the present disclosure, the vector is an adeno-associated virus vector, a lentiviral vector, a ribonucleoprotein complex, or a virus-like particle.

In some embodiments of the present disclosure:
when the vector is an adeno-associated virus vector, the adeno-associated virus vector is a recombinant adeno-associated virus vector of serotype AAV1, AAV2, AAV4, AAV5, AAV6, AAV7, AAVrh74, AAV8, AAV9, AAV10, AAV11, AAV12, or AAV13;
when the vector is a lentiviral vector, the lentiviral vector is pseudotyped with an envelope protein; optionally, the isolated nucleic acid is linked to an aptamer sequence;
when the vector is a virus-like particle, the isolated nucleic acid is linked to a gene encoding a gag protein.

The present disclosure provides, in another aspect, a delivery system, wherein the delivery system comprises:
(1) a delivery tool, and
(2) a Cas12 protein as described in the present disclosure, a fusion protein or conjugate as described in the present disclosure, or a nucleic acid as described in the present disclosure; a CRISPR-Cas12 system as described in the present disclosure; or a vector system as described in the present disclosure.

In some embodiments of the present disclosure, the delivery tool is a lipid nanoparticle, a nanoparticle, a liposome, an exosome, a microbubble, or a gene gun.

In some embodiments of the present disclosure, the delivery tool is a lipid nanoparticle, wherein the lipid nanoparticle comprises the guide polynucleotide and an mRNA encoding the Cas12 protein or the fusion protein or conjugate.

The present disclosure provides, in another aspect, a cell, wherein the cell comprises a Cas12 protein as described in the present disclosure, a fusion protein or conjugate as described in the present disclosure, an isolated nucleic acid as described in the present disclosure, a CRISPR-Cas12 system as described in the present disclosure, or a vector system as described in the present disclosure.

In some embodiments of the present disclosure, the cell is a eukaryotic cell.

In some embodiments of the present disclosure, the eukaryotic cell is a mammalian cell.

The present disclosure provides, in another aspect, a pharmaceutical composition, wherein the pharmaceutical composition comprises a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, or a cell as described in the present disclosure.

In some embodiments of the present disclosure, the pharmaceutical composition comprises a pharmaceutically acceptable excipient.

The present disclosure provides, in another aspect, a kit, wherein the kit comprises a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, or a cell as described in the present disclosure.

The present disclosure provides, in another aspect, use of a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, a cell as described in the present disclosure, a pharmaceutical composition as described in the present disclosure, or a kit as described in the present disclosure, in the manufacture of a reagent or medicament for diagnosing, treating, and/or preventing a disease or condition associated with a target nucleic acid.

In some embodiments of the present disclosure, the reagent or medicament is used to: cleave one or more target nucleic acid molecules or introduce a nick into one or more target nucleic acid molecules; activate or upregulate expression of one or more target nucleic acid molecules; activate or inhibit transcription of one or more target nucleic acid molecules; inactivate one or more target nucleic acid molecules; visualize, label, or detect one or more target nucleic acid molecules; bind one or more target nucleic acid molecules; transport one or more target nucleic acid molecules; and mask one or more target nucleic acid molecules.

The present disclosure provides, in another aspect, a method for detecting, binding, or cleaving a target nucleic acid, wherein the method comprises contacting the target nucleic acid with a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, a cell as described in the present disclosure, a pharmaceutical composition as described in the present disclosure, or a kit as described in the present disclosure.

In some embodiments of the present disclosure, the method is a method for non-diagnostic and/or non-therapeutic purposes; and/or the fusion protein or conjugate comprises a detectable label, such as a label detectable by fluorescence, Southern blotting, or FISH.

The present disclosure provides, in another aspect, a method for altering a cell state, wherein the method comprises contacting a cell with a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, a cell as described in the present disclosure, a pharmaceutical composition as described in the present disclosure, or a kit as described in the present disclosure, thereby altering the cell state.

In some embodiments of the present disclosure, the method results in one or more of: (i) inducing cellular senescence in vitro or in vivo; (ii) inducing cell cycle arrest in vitro or in vivo; (iii) inhibiting cell growth in vitro or in vivo and/or suppressing cell growth in vitro or in vivo; (iv) inducing unresponsiveness (anergy) in vitro or in vivo; (v) inducing apoptosis in vitro or in vivo; and (vi) inducing necrosis in vitro or in vivo.

In some embodiments of the present disclosure, the method is a method for non-diagnostic and/or non-therapeutic purposes.

The present disclosure provides, in another aspect, a method for diagnosing, treating, and/or preventing a disease or condition associated with a target nucleic acid, comprising administering, to a sample obtained from a subject in need thereof or to a subject in need thereof, a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, a cell as described in the present disclosure, a pharmaceutical composition as described in the present disclosure, or a kit as described in the present disclosure.

The present disclosure provides, in another aspect, a Cas12 protein as described in the present disclosure, a fusion protein or conjugate as described in the present disclosure, an isolated nucleic acid as described in the present disclosure, a CRISPR-Cas12 system as described in the present disclosure, a vector system as described in the present disclosure, a delivery system as described in the present disclosure, a cell as described in the present disclosure, a pharmaceutical composition as described in the present disclosure, or a kit as described in the present disclosure, for diagnosing, treating, and/or preventing a disease or condition associated with a target nucleic acid.

Based on common knowledge in the art, the above preferred conditions can be combined arbitrarily to obtain preferred embodiments of the present disclosure.

The reagents and raw materials used in the present disclosure are all commercially available.

The advantageous technical effects of the present disclosure include:

In some embodiments, the present disclosure improves gene-editing efficiency in mammalian cells by introducing rational and non-rational mutations into the amino acid sequence of the wild-type Cas12 protein as set forth in SEQ ID NO: 1.

Through bioinformatics analysis and experimental validation, the present disclosure screens and obtains a new Cas protein having DNA cleavage activity, named C12-102, which has an amino acid sequence length of 1112 aa and is relatively shorter than the currently commonly used SpCas9 protein (1368 aa) and AsCpf1 protein (1307 aa), and is easier to be packaged into small-capacity gene therapy vectors (e.g., AAV).

Many Cas12 PAM sequences contain two or more specific bases and are rich in T (e.g., TTTN or TTN), whereas the PAM sequence of C12-102 is a single A base; therefore, C12-102 can be used to edit many target sequences that were previously difficult to edit, greatly expanding the editable range.

In addition, through bioinformatics analysis and prediction of C12-102 and Cas12-Y2, the present disclosure performs wet-lab testing of mutants at certain positions of the amino acid sequences, thereby obtaining a series of mutants.

### BRIEF DESCRIPTION OF THE DRAWINGS

FIG. 1 is a map of the pCDH-CMV-EGFP-Reporter3-EF1a-Puro plasmid.

FIG. 2 is an SDS-PAGE gel electrophoresis image of the C12-102 recombinant protein.

FIG. 3 is a schematic diagram of the C12-102 targeting template sequence used for PAM recognition.

FIG. 4 shows 7-nt random sequences recognized using C12-102-sgRNA.

FIG. 5 shows 7-nt random sequences recognized using C12-102-sgRNA-Rev.

FIG. 6 is a gel electrophoresis detection image of dsDNA cleavage by C12-102.

FIG. 7 shows fluorescence assay results of ssDNA cleavage by C12-102.

FIG. 8 shows the bilobed structure of C12-102, including the recognition (REC) lobe and the nuclease (NUC) lobe.

### DETAILED DESCRIPTION OF THE INVENTION

In In the present disclosure, unless otherwise specified, the scientific and technical terms used herein have the meanings commonly understood by a person skilled in the art. In addition, the operational steps in molecular genetics, nucleic acid chemistry, chemistry, molecular biology, biochemistry, cell culture, microbiology, cell biology, genomics, recombinant DNA, and the like used herein are conventional steps widely used in the corresponding fields. Meanwhile, for better understanding of the present disclosure, definitions and explanations of relevant terms are provided below.

In the present disclosure, “more” can refer to two or more than two.

In the present disclosure, letters in amino acid sequences represent single-letter abbreviations of amino acids known in the art, for example, as described in J. Biol. Chem., 243, p. 3558 (1968): alanine: Ala-A, arginine: Arg-R, aspartic acid: Asp-D, cysteine: Cys-C, glutamine: Gln-Q, glutamic acid: Glu-E, histidine: His-H, glycine: Gly-G, asparagine: Asn-N, tyrosine: Tyr-Y, proline: Pro-P, serine: Ser-S, methionine: Met-M, lysine: Lys-K, valine: Val-V, isoleucine: Ile-I, phenylalanine: Phe-F, leucine: Leu-L, tryptophan: Trp-W, and threonine: Thr-T.

In the present disclosure, “comprises or consists of” or “includes or consists of” means that the technical solution is expressed in both an open-ended manner and a closed-ended manner. For example, “the amino acid sequence of the Cas12 protein comprises or consists of an amino acid sequence that, as compared with SEQ ID NO: 18, has an amino acid difference at position S211” includes an open-ended expression of “the Cas12 protein comprises an amino acid sequence that, as compared with SEQ ID NO: 18, has a difference at position S211”, and a closed-ended expression of “the amino acid sequence of the Cas12 protein, as compared with the amino acid sequence set forth in SEQ ID NO: 18, differs only at position S211”.

In the present disclosure, “amino acid difference” refers to a difference of an amino acid residue at a specific position in the amino acid sequence of a protein, including substitution, addition, or deletion.

As is well known to those skilled in the art, in a protein or peptide, two adjacent amino acids each lose one OH or H and undergo dehydration condensation to form a peptide bond, and each amino acid actually exists in the form of an amino acid residue. Therefore, in the present disclosure, the terms “amino acid” and “amino acid residue” generally represent the same meaning. In addition, for simplicity, in the present disclosure the amino acid residue before substitution is retained before the position number: the letter before the position indicates the original amino acid residue, the letter after the position indicates the substituted amino acid residue, and “A” indicates that the original amino acid residue does not exist. For example, S211 indicates that the original amino acid residue at position 211 is S; when it is substituted with R, it can be denoted as S211R.

In the present disclosure, the number indicated for a position refers to the position of the amino acid residue corresponding to the amino acid sequence of the Cas12 protein or Cas12 protein mutant in SEQ ID NO: 1, SEQ ID NO: 18, or SEQ ID NO: 40.

In the present disclosure, when an amino acid is substituted, it means that it is substituted with another amino acid residue different from the original amino acid residue. If the original amino acid residue is a positively charged amino acid and it is substituted with a positively charged amino acid, it means that it is substituted with another positively charged amino acid residue different from the original amino acid residue. For example, when the original amino acid residue is R and it is substituted with a positively charged amino acid, it means that it is substituted with H or K.

In some embodiments of the present disclosure, the Cas12 protein, Cas12 mutant, Cas12 inactive variant, Cas12 fusion protein, or Cas12 conjugate can form a CRISPR complex with a guide polynucleotide. In some embodiments of the present disclosure, the Cas12 protein, Cas12 mutant, Cas12 inactive variant, Cas12 fusion protein, or Cas12 conjugate can form a CRISPR complex with a guide polynucleotide, wherein the guide polynucleotide guides the CRISPR complex to bind to a target nucleic acid in a sequence-specific manner. In some embodiments of the present disclosure, the Cas12 protein, Cas12 mutant, Cas12 inactive variant, Cas12 fusion protein, or Cas12 conjugate can form a CRISPR complex with a guide polynucleotide, wherein the guide polynucleotide comprises a guide sequence, and the guide sequence is engineered to guide the CRISPR complex to bind to a target nucleic acid in a sequence-specific manner. In some embodiments of the present disclosure, the Cas12 protein, Cas12 mutant, Cas12 inactive variant, Cas12 fusion protein, or Cas12 conjugate can form a CRISPR complex with a guide polynucleotide, wherein the guide polynucleotide guides the CRISPR complex to bind to and cleave a target nucleic acid in a sequence-specific manner. Optionally, the target nucleic acid is a single-stranded nucleic acid or a double-stranded nucleic acid; optionally, the target nucleic acid is a single-stranded DNA or a double-stranded DNA; optionally, cleaving the target nucleic acid refers to cleaving only one strand of a double-stranded nucleic acid, or refers to cleaving two strands of a double-stranded nucleic acid; optionally, cleaving the target nucleic acid refers to cleaving only one strand of a double-stranded DNA, or refers to cleaving two strands of a double-stranded DNA. In some embodiments of the present disclosure, the Cas12 protein, Cas12 mutant, Cas12 inactive variant, Cas12 fusion protein, or Cas12 conjugate can form a CRISPR complex with a guide polynucleotide, wherein the guide polynucleotide guides the CRISPR complex to bind to a target nucleic acid in a sequence-specific manner and causes base conversion of at least one base in the target nucleic acid. In some embodiments of the present disclosure, the Cas12 protein, Cas12 mutant, Cas12 inactive variant, Cas12 fusion protein, or Cas12 conjugate can form a CRISPR complex with a guide polynucleotide, wherein the guide polynucleotide guides the CRISPR complex to bind to a target nucleic acid in a sequence-specific manner and regulates expression of at least one gene on the target nucleic acid. Optionally, the at least one base is 1 base, 2 bases, 3 bases, 4 bases, 5 bases, 6 bases, 7 bases, 8 bases, 9 bases, or 10 bases. Optionally, the at least one gene is 1 gene, 2 genes, 3 genes, 4 genes, 5 genes, 6 genes, 7 genes, 8 genes, 9 genes, or 10 genes.

### Sequence Identity

As used herein, the term “sequence identity” (identity or percent identity) refers to the degree of sequence match between two polypeptides or between two nucleic acids. When a position in each of two sequences being compared is occupied by the same base or amino-acid monomeric subunit (e.g., when a given position in each of two DNA molecules is occupied by adenine, or when a given position in each of two polypeptides is occupied by lysine), the molecules are identical at that position. The “percent sequence identity” (percent identity) between two sequences is a function of the number of matching positions shared by the two sequences divided by the number of positions compared, multiplied by 100%. For example, if 6 positions out of 10 positions in two sequences match, the two sequences have 60% sequence identity. Generally, the comparison is made by aligning two sequences to achieve maximum sequence identity. Such alignment can be performed using publicly available and commercially available alignment algorithms and programs, including, but not limited to, Clustal 22, MAFFT, Probcons, T-Coffee, Probalign, and BLAST, as can be reasonably selected by one of ordinary skill in the art. One of ordinary skill in the art can determine suitable parameters for sequence alignment, including any algorithm required to achieve optimal alignment or best comparison over the full length of the sequences being compared, or any algorithm required to achieve optimal alignment or best comparison over a local region of the sequences being compared.

### CRISPR-Cas12 System

As used herein, the terms “Clustered Regularly Interspaced Short Palindromic Repeats (CRISPR)-CRISPR-associated (Cas) (CRISPR-Cas) system” and “CRISPR system” are used interchangeably and have the meaning ordinarily understood by those skilled in the art. Such systems generally comprise transcription products or other elements associated with expression of CRISPR-associated (“Cas”) genes, or transcription products or other elements capable of directing activities of the Cas genes. Such transcription products or other elements may comprise sequences encoding Cas effector proteins and guide polynucleotides.

In 2015, the Feng Zhang laboratory discovered Cas12a and classified it as Type V of the Class II CRISPR-Cas system. After detailed studies of the V-A subtype (Cas12a), the Feng Zhang laboratory also reported Cas12b (C2C1) in 2015. In 2017, Burstein et al. reported the Cas12e (CasX) nuclease. In 2019, Winston X. Yan et al. reported, in detail through bioinformatics analyses, newly discovered Type V Cas effector proteins Cas12c, Cas12h, Cas12i, and Cas12g.

In some embodiments, the Cas12 protein described herein refers to a protein whose amino acid sequence comprises or consists of a protein having at least 50%, at least 80%, at least 85%, at least 90%, at least 95%, at least 96%, at least 97%, at least 98%, or at least 99% sequence identity to SEQ ID NO: 1. When the CRISPR-Cas12 system comprises a fusion protein or conjugate comprising the Cas12 protein and a protein domain, the percent sequence identity between the Cas12 portion of the fusion protein or conjugate and the reference sequence is calculated.

In the present disclosure, the CRISPR-Cas12 system comprises a Cas12 protein having at least 50% sequence identity to SEQ ID NO: 1, or a nucleic acid encoding the Cas12 protein, and a guide polynucleotide or a nucleic acid encoding the guide polynucleotide, wherein the guide polynucleotide comprises a direct repeat sequence linked to a guide sequence, the guide sequence is engineered to hybridize with a target DNA, and the guide polynucleotide can form a CRISPR complex with the Cas12 protein and guide the CRISPR complex to bind to the target DNA in a sequence-specific manner.

In some embodiments, the Cas12 protein described herein refers to a protein whose amino acid sequence comprises or consists of a protein having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to SEQ ID NO: 18. The Cas12 protein mutant described herein refers to a protein whose amino acid sequence comprises or consists of a protein having at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 40. When the CRISPR-Cas12 system comprises a Cas12 fusion protein or conjugate comprising the Cas12 protein or Cas12 protein mutant and a protein domain, the percent sequence identity between the Cas12 portion of the Cas12 fusion protein or conjugate and the reference sequence is calculated.

In the present disclosure, the CRISPR-Cas12 system comprises a Cas12 protein having at least 50% sequence identity to SEQ ID NO: 18 or a Cas12 protein mutant having at least 70% sequence identity to SEQ ID NO: 40, or nucleic acids encoding them, and a guide polynucleotide or a nucleic acid encoding the guide polynucleotide, wherein the guide polynucleotide comprises a direct repeat sequence linked to a guide sequence, the guide sequence is engineered to hybridize with a target nucleic acid, and the guide polynucleotide can form a complex with the Cas12 protein or Cas12 protein mutant and guide the complex to bind to the target nucleic acid in a sequence-specific manner.

### Guide Polynucleotide

As used herein, the term “guide polynucleotide” refers to a molecule in a CRISPR-Cas system that forms a CRISPR complex with a Cas protein and guides the CRISPR complex to a target sequence. Generally, a guide polynucleotide comprises a scaffold sequence linked to a guide sequence, wherein the guide sequence can hybridize with the target sequence. The scaffold sequence generally comprises a direct repeat sequence, and may sometimes further comprise a tracrRNA sequence. In the Cas12-based CRISPR system described in the present disclosure, a tracrRNA sequence is not required.

In some embodiments, the guide polynucleotide of the CRISPR-Cas12 system is guide DNA. In some embodiments, the guide polynucleotide is a chemically modified guide polynucleotide. In some embodiments, the guide polynucleotide comprises at least one chemically modified nucleotide.

In some embodiments, the guide polynucleotide comprises at least one guide sequence (guide sequence, also referred to as a spacer sequence) linked to at least one direct repeat sequence (direct repeat, DR). In some embodiments, the guide sequence is located at the 3′ end of the direct repeat sequence. In some embodiments, the guide sequence is located at the 5′ end of the direct repeat sequence.

In some embodiments, the guide sequence comprises at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides. In some embodiments, the guide sequence comprises no more than 60 nucleotides, no more than 55 nucleotides, no more than 50 nucleotides, no more than 45 nucleotides, no more than 40 nucleotides, no more than 35 nucleotides, or no more than 30 nucleotides. In some embodiments, the guide sequence comprises 15-20 nucleotides, 20-25 nucleotides, 25-30 nucleotides, 30-35 nucleotides, or 35-40 nucleotides.

In some embodiments, the guide sequence has sufficient complementarity to the target DNA sequence to hybridize with the target DNA and guide sequence-specific binding of the CRISPR-Cas12 complex to the target DNA. In some embodiments, the guide sequence has 100% complementarity to the target DNA (or a region of the DNA to be targeted), but the guide sequence may have less than 100% complementarity to the target DNA, for example, at least 80%, at least 85%, at least 90%, at least 95%, at least 98%, or at least 99% complementarity.

In some embodiments, the guide sequence is engineered to hybridize with the target DNA with no more than two nucleotide mismatches. In some embodiments, the guide sequence is engineered to hybridize with the target DNA with no more than one nucleotide mismatch. In some embodiments, the guide sequence is engineered to hybridize with the target DNA with or without mismatches.

In some embodiments, the direct repeat sequence comprises at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, at least 30 nucleotides, at least 31 nucleotides, at least 32 nucleotides, at least 33 nucleotides, at least 34 nucleotides, at least 35 nucleotides, or at least 36 nucleotides. In some embodiments, the direct repeat sequence comprises no more than 60 nucleotides, no more than 55 nucleotides, no more than 50 nucleotides, no more than 45 nucleotides, no more than 40 nucleotides, or no more than 35 nucleotides. In some embodiments, the direct repeat sequence comprises 20-25 nucleotides, 25-30 nucleotides, 30-35 nucleotides, or 35-40 nucleotides.

In some embodiments, the direct repeat sequence has at least 60%, at least 70%, at least 80%, at least 85%, at least 90%, at least 91%, at least 92%, at least 93%, at least 94%, at least 95%, at least 96%, at least 97%, at least 98%, or at least 99% sequence identity to SEQ ID NO: 17 or SEQ ID NO: 26.

In some embodiments, the CRISPR-Cas12 system comprises at least 2, at least 3, at least 4, at least 5, at least 10, or at least 20 different guide polynucleotides. In some embodiments, the guide polynucleotides target at least 2, at least 3, at least 4, at least 5, at least 10, or at least 20 different target DNA molecules, or target at least 2, at least 3, at least 4, at least 5, at least 10, or at least 20 different regions of one or more target DNA molecules.

In some embodiments, the guide polynucleotide comprises a constant direct repeat sequence located upstream of a variable guide sequence. In some embodiments, multiple guide polynucleotides are part of an array (which may be part of a vector, such as a viral vector or a plasmid). For example, a guide array comprising the sequence DR-spacer-DR-spacer-DR-spacer can comprise three unique unprocessed guide polynucleotides (one for each DR-spacer sequence). Once introduced into a cell or cell-free system, the array is processed by the Cas12 protein into three separate mature guide polynucleotides. This allows multiplexing, for example, delivering multiple guide polynucleotides to a cell or system to target multiple target DNAs or multiple regions within a single target DNA.

The ability of a guide polynucleotide to guide sequence-specific binding of a CRISPR complex to a target DNA can be evaluated by any suitable assay. For example, components of a CRISPR system sufficient to form a CRISPR complex, including the guide polynucleotide to be tested, can be provided to a host cell comprising the corresponding target DNA molecule, for example, by transfection with a vector encoding components of the CRISPR complex, and then preferential cleavage within the target sequence is assessed. Similarly, cleavage of a target DNA sequence can be evaluated in vitro by providing a target DNA and components of the CRISPR complex, including the guide polynucleotide to be tested and a control guide polynucleotide different from the guide polynucleotide being tested, and comparing the ability to bind the target DNA or the rate of cleavage of the target DNA between the guide polynucleotide to be tested and the control guide polynucleotide. The ability of a CRISPR complex to cleave a target nucleic acid or target DNA can also be evaluated by the assays described above.

### Cas12 Mutants

In some embodiments, as compared with the wild-type Cas12 protein (SEQ ID NO: 1, SEQ ID NO: 18, or SEQ ID NO: 40), the Cas12 protein provided herein comprises one or more mutations, such as a single amino-acid insertion, a single amino-acid deletion, a single amino-acid substitution, or a combination thereof. In some examples, as compared with the wild-type Cas12 protein (SEQ ID NO: 1, SEQ ID NO: 18, or SEQ ID NO: 40), the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 amino-acid changes (e.g., insertion, deletion, or substitution) while retaining the ability to bind a target DNA molecule complementary to the guide sequence of a guide polynucleotide, and/or retaining the ability to process a guide-array RNA transcript into guide polynucleotides. In some examples, as compared with the wild-type Cas12 protein (SEQ ID NO: 1, SEQ ID NO: 18, or SEQ ID NO: 40), the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 amino-acid changes (e.g., insertion, deletion, or substitution) while retaining the ability to bind a target DNA molecule complementary to the guide sequence of a guide polynucleotide. In some examples, as compared with the wild-type Cas12 protein (SEQ ID NO: 1, SEQ ID NO: 18, or SEQ ID NO: 40), the Cas12 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino-acid changes (e.g., insertion, deletion, or substitution) while retaining the ability to bind a target DNA molecule complementary to the guide sequence of a guide polynucleotide, and/or retaining the ability to process a guide-array RNA transcript into guide polynucleotides.

One type of modification or mutation comprises substituting an amino-acid residue with an amino-acid residue having similar biochemical properties, i.e., a conservative substitution (e.g., 1-4, 1-8, 1-10, or 1-20 conservative substitutions). Generally, conservative substitutions have little or no effect on the activity of the resulting protein or peptide. For example, a conservative substitution is an amino-acid substitution in a Cas12 protein that does not substantially affect binding between the Cas12 protein and a target DNA molecule complementary to the guide sequence of a gRNA molecule, and/or processing of a guide-array RNA transcript into gRNA molecules.

More substantial changes may be made by using less conservative substitutions, for example, by selecting residues that differ more in maintaining the following effects: (a) the structure of the polypeptide backbone in the region where the substitution occurs, e.g., as a helical or folded conformation; (b) the charge or hydrophobicity of a region that interacts with a target site; or (c) the bulk of a side chain. Substitutions expected to produce the greatest changes in polypeptide function are typically (a) substitutions between a hydrophilic residue (e.g., serine or threonine) and a hydrophobic residue (e.g., leucine, isoleucine, phenylalanine, valine, or alanine); (b) substitutions between cysteine or proline and any other residue; (c) substitutions between a residue having a positively charged side chain (e.g., lysine, arginine, or histidine) and a residue having a negatively charged side chain (e.g., glutamic acid or aspartic acid); or (d) substitutions between a residue having a bulky side chain (e.g., phenylalanine) and a residue lacking a side chain (e.g., glycine).

### Cas12 Active Fragments

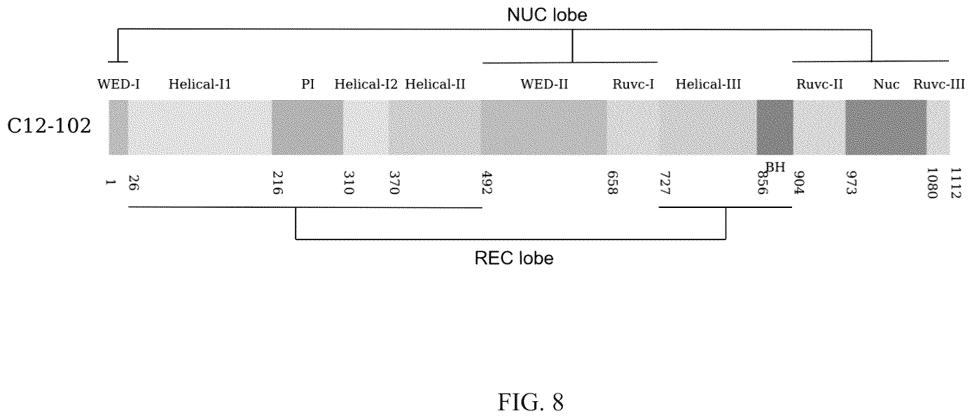

In the present disclosure, the bilobed structure of the Cas12 protein C12-102 is shown in FIG. 8 (and FIG. 8 is also applicable to the structures of C12-102 protein mutants described herein). The numbers in FIG. 8 refer to the amino-acid positions, in SEQ ID NO: 18, corresponding to the WED-I domain, Helical-I1 domain, PI domain, Helical-12 domain, Helical-II domain, WED-II domain, RuvC-I domain, Helical-III domain, BH domain, RuvC-II domain, Nuc domain, and RuvC-III domain described in the present disclosure. With respect to the domain boundaries of SEQ ID NO: 40 and its mutants, the corresponding positions relative to the boundaries shown in FIG. 8 can be determined by sequence alignment with the C12-102 protein, thereby obtaining the domain boundaries.

The Cas12 protein described in the present disclosure, in addition to comprising the domains corresponding to the Cas12 active fragment, may further comprise other domains of Cas12 proteins known in the prior art, which together form the complete Cas12 protein structure as shown in FIG. 8 to achieve the functions of the Cas12 protein described herein, including, but not limited to, retaining the ability of the Cas12 protein to bind a target nucleic acid molecule complementary to the guide sequence of a guide polynucleotide, and/or retaining the ability to process a guide-sequence RNA transcript into guide polynucleotide molecules.

### Cas12 Inactive Variants

By introducing point mutations to inactivate the RuvC domain of Cas12, the Cas12 protein loses endonuclease activity, and the resulting dCas12 can only bind a target gene under the mediation of a guide polynucleotide, without having DNA cleavage activity.

Point mutations can also be introduced to partially inactivate the RuvC domain of Cas12 to form a Cas12 nickase (nickase Cas12, nCas12), which binds a target gene under the mediation of a guide polynucleotide and cleaves one single strand of a double-stranded nucleic acid without cleaving the other strand.

Accordingly, dCas12 or nCas12 can be fused with other domains (including, but not limited to, a deaminase domain, a transcription activation domain, a transcription repression domain, a methylation domain, a demethylation domain, a histone acetylation domain, and a histone deacetylation domain), and guided to a target sequence of a target nucleic acid by a guide polynucleotide, such that the corresponding function is carried out by the other domain; for example, achieving a C→T base conversion by deamination of a cytosine base, achieving an A→G base conversion by deamination of an adenine base, achieving transcriptional repression by the KRAB transcription repression domain, and promoting transcription by the VP64 transcription activation domain.

### Subcellular Localization Signals

In some embodiments, the Cas12 protein is fused with at least one homologous or heterologous subcellular localization signal. Exemplary subcellular localization signals include organelle localization signals, such as a nuclear localization signal (NLS), a nuclear export signal (NES), or a mitochondrial localization signal.

### Protein Domains

In some embodiments, the Cas12 protein or Cas12 protein mutant is covalently linked or fused to a homologous or heterologous protein domain.

In some embodiments, the protein domain is optionally selected from one or more of the following: a DNA-binding domain, a protease domain, a transcription activation domain, a transcription repression domain, a nuclease domain (including a polypeptide having ssDNA cleavage activity and/or a polypeptide having dsDNA cleavage activity), a deaminase domain, a uracil DNA glycosylase domain (UDG), a uracil DNA glycosylase inhibitor domain (UGI), a methyltransferase, a demethylase, a transcription release factor, a histone acetyltransferase domain, a histone deacetylase domain, a DNA ligase, an epitope tag, and a reporter domain.

In some embodiments, the Cas12 protein or Cas12 protein mutant optionally comprises 0, 1, 2, 3, or more protein domains at the N-terminus and/or the C-terminus.

### Vector Systems

In another aspect, the present disclosure relates to a vector system comprising the CRISPR-Cas12 system described herein, wherein the vector system comprises one or more vectors, and the vectors comprise a polynucleotide sequence encoding the Cas12 protein and a polynucleotide sequence encoding the guide polynucleotide.

In some embodiments, the vector system comprises at least one plasmid or viral vector (e.g., retrovirus, lentivirus, adenovirus, adeno-associated virus, or herpes simplex virus). In some embodiments, the polynucleotide sequence encoding the Cas12 protein and the polynucleotide sequence encoding the guide polynucleotide are located on the same vector. In some embodiments, the polynucleotide sequence encoding the Cas12 protein and the polynucleotide sequence encoding the guide polynucleotide are located on multiple vectors.

In some embodiments, the polynucleotide sequence encoding the Cas12 protein and/or the polynucleotide sequence encoding the guide polynucleotide is operably linked to a regulatory element. Regulatory elements include promoters, enhancers, internal ribosome entry sites (IRESs), and other expression control elements (e.g., transcription termination signals such as polyadenylation signals and poly-U sequences). Regulatory elements include regulatory elements that direct constitutive expression of a nucleotide sequence in many types of host cells, as well as regulatory elements that direct expression of a nucleotide sequence only in certain host cells (e.g., tissue-specific regulatory sequences). Tissue-specific promoters can drive expression primarily in a desired tissue of interest, for example, muscle, neurons, bone, skin, blood, a specific organ (e.g., liver or pancreas), or a specific cell type (e.g., lymphocytes). Regulatory elements can also direct expression in a time-dependent manner, for example, in a cell-cycle-dependent or developmental-stage-dependent manner, which may or may not be tissue- or cell-type-specific. In some embodiments, the regulatory element is an enhancer element, such as WPRE, a CMV enhancer, the R-U5 region of the HTLV-1 LTR, an SV40 enhancer, or an intron sequence between exons 2 and 3 of rabbit β-globin.

In some embodiments, the vector comprises a pol III promoter (e.g., a U6 or H1 promoter), a pol II promoter (e.g., a retroviral Rous sarcoma virus (RSV) LTR promoter (optionally with an RSV enhancer), a cytomegalovirus (CMV) promoter (optionally with a CMV enhancer), an SV40 promoter, a dihydrofolate reductase promoter, a β-actin promoter, a phosphoglycerate kinase (PGK) promoter, or an EF1α promoter), or a pol III promoter and a pol II promoter.

In some embodiments, the promoter is a constitutive promoter that is continuously active and is not regulated by an external signal or molecule. Suitable constitutive promoters include, but are not limited to, CMV, RSV, SV40, EF1α, CAG, and β-actin promoters. In some embodiments, the promoter is an inducible promoter regulated by an external signal or molecule (e.g., a transcription factor).

In some embodiments, the promoter is a tissue-specific promoter that can be used to drive tissue-specific expression of the Cas12 protein. Suitable muscle-specific promoters include, but are not limited to, CK8, MHCK7, a myoglobin promoter (Mb), a desmin promoter, a muscle creatine kinase promoter (MCK) and variants thereof, and an SPc5-12 synthetic promoter. Suitable immune-cell-specific promoters include, but are not limited to, a B29 promoter (B cells), a CD14 promoter (monocytes), a CD43 promoter (leukocytes and platelets), CD68 (macrophages), and an SV40/CD43 promoter (leukocytes and platelets). Suitable blood-cell-specific promoters include, but are not limited to, a CD43 promoter (leukocytes and platelets), a CD45 promoter (hematopoietic cells), INF-β (hematopoietic cells), a WASP promoter (hematopoietic cells), an SV40/CD43 promoter (leukocytes and platelets), and an SV40/CD45 promoter (hematopoietic cells). Suitable pancreas-specific promoters include, but are not limited to, an elastase-1 promoter. Suitable endothelial-cell-specific promoters include, but are not limited to, a Fit-1 promoter and an ICAM-2 promoter. Suitable neuronal tissue/cell-specific promoters include, but are not limited to, a GFAP promoter (astrocytes), a SYN1 promoter (neurons), and an NSE/RU5′ promoter (mature neurons). Suitable kidney-specific promoters include, but are not limited to, an NphsI promoter (podocytes). Suitable bone-specific promoters include, but are not limited to, an OG-2 promoter (osteoblasts and odontoblasts). Suitable lung-specific promoters include, but are not limited to, an SP-B promoter (lung). Suitable liver-specific promoters include, but are not limited to, an SV40/Alb promoter. Suitable heart-specific promoters include, but are not limited to, α-MHC.

### AAV Vectors

In another aspect, the present disclosure relates to an adeno-associated virus (AAV) vector comprising the CRISPR-Cas12 system described herein, wherein the AAV vector comprises DNA encoding the Cas12 protein and the guide polynucleotide described herein.

Delivery of a CRISPR-Cas system by an AAV vector is described in Maeder et al., Nature Medicine 25:229-233 (2019), which clinically demonstrated the safety and efficacy of subretinal delivery of AAV. Local delivery by subretinal injection, the natural tropism of AAV5 for photoreceptor cells, and use of a photoreceptor-specific GRK1 promoter were used to restrict expression of the CRISPR/Cas system to the therapeutic target tissue and cell types, which is incorporated herein by reference in its entirety. In some embodiments, the AAV vector comprises an ssDNA genome, wherein the genome comprises encoding sequences of the Cas12 protein and the guide polynucleotide flanked by ITRs.

In some embodiments, the CRISPR-Cas12 system described herein is packaged in an AAV vector, such as AAV1, AAV2, AAV3, AAV4, AAV5, AAV6, AAV7, AAV8, AAV9, and AAVrh74. In some embodiments, the CRISPR-Cas12 system described herein is packaged in an AAV vector comprising an engineered capsid having tissue tropism, such as an engineered muscle-tropic capsid. Tabebordbar et al., Cell 184:4919-4938 (2021) described engineering AAV capsids with tissue tropism by directed evolution and identified a class of capsids containing an RGD motif; systemic injection of MyoAAV efficiently transduced primate muscle. This reference is incorporated herein by reference in its entirety.

### Lipid Nanoparticles

In another aspect of the present disclosure, lipid nanoparticles (LNPs) comprising the CRISPR-Cas12 system described herein are provided, wherein the LNP comprises the guide polynucleotide described herein and an mRNA encoding the Cas12 protein described herein.

Gillmore et al., N. Engl. J. Med., 385:493-502 (2021) describes LNP delivery of a CRISPR-Cas system, wherein the lipid nanoparticles (LNPs) are composed of four lipids, including a proprietary ionizable lipid LP000001; DSPC; cholesterol; and DMG-PEG2k; and the LNP suspension is formulated in an aqueous buffer of Tris, NaCl, and sucrose at pH 7.4, the entire disclosure of which is incorporated herein by reference. In some embodiments, in addition to the RNA payload (Cas12 mRNA and the guide polynucleotide), the lipid nanoparticles (LNPs) further comprise four components: a cationic or ionizable lipid, cholesterol, a helper lipid, and a PEG-lipid. In some embodiments, the cationic or ionizable lipid comprises cKK-E12, C12-200, ALC-0315, DLin-MC3-DMA, DLin-KC2-DMA, FTT5, Moderna SM-102, and Intellia LP01. In some embodiments, the PEG-lipid comprises PEG-2000-C-DMG, PEG-2000-DMG, or ALC-0159. In some embodiments, the helper lipid comprises DSPC. The components of LNPs are described in Paunovska et al., Nature Reviews Genetics 23:265-280 (2022). Variants of FDA-approved LNPs contain four basic components: a cationic or ionizable lipid, cholesterol, a helper lipid, and a polyethylene glycol (PEG) lipid, the entire disclosure of which is incorporated herein by reference.

### Lentiviral Vectors

In another aspect of the present disclosure, lentiviral vectors comprising the CRISPR-Cas12 system described herein are provided, wherein the lentiviral vector comprises the guide polynucleotide described herein and an mRNA encoding the Cas12 protein described herein. In some embodiments, the lentiviral vector is pseudotyped with a homologous or heterologous envelope protein such as VSV-G.

### RNP Complexes

In another aspect of the present disclosure, ribonucleoprotein complexes comprising the CRISPR-Cas12 system described herein are provided, wherein the ribonucleoprotein complex is formed by the guide polynucleotide described herein and the Cas12 protein. In some embodiments, the ribonucleoprotein complex can be delivered to a eukaryotic cell by microinjection or electroporation.

### Virus-Like Particles

In another aspect of the present disclosure, virus-like particles (VLPs) comprising the CRISPR-Cas12 system described herein are provided, wherein the virus-like particle comprises the guide polynucleotide described herein and the Cas12 protein, or a ribonucleoprotein complex composed of the guide polynucleotide and the Cas12 protein.

Banskota et al. Cell 185 (2): 250-265 (2022) reported the development and application of DNA-free engineered virus-like particles (eVLPs) for efficient packaging and delivery of a base editor or a Cas9 ribonucleoprotein; Mangeot et al., Nature Communications 10 (1): 1-15 (2019) used engineered murine leukemia virus-like particles (Nanoblades) loaded with a Cas9-sgRNA ribonucleoprotein to induce efficient genome editing in cell lines and primary cells (including human induced pluripotent stem cells, human hematopoietic stem cells, and murine bone marrow cells); Campbell et al., Molecular Therapy 27:151-163 (2019) used specialized extracellular vesicles termed “gesicles” to efficiently but transiently deliver Cas9, in ribonucleoprotein form, targeting the HIV long terminal repeat (LTR), wherein gesicles are produced by expression of vesicular stomatitis virus glycoprotein and a packaging protein (as cargo), thereby avoiding transgene delivery and enabling finer control of Cas9 expression; and Mangeot et al., Molecular Therapy 19 (9): 1656-1666 (2011) reported that overexpression of the vesicular stomatitis virus (VSV-G) spike glycoprotein in human cells induced the release of fusogenic vesicles termed gesicles, and biochemical and functional studies showed that gesicles incorporate proteins from the producer cells and can deliver such proteins to recipient cells, and this protein transduction approach allows direct transport of cytosolic, nuclear, or surface proteins in target cells. These references each describe engineered VLPs, the entire disclosures of which are incorporated herein by reference.

In some embodiments, engineered virus-like particles (VLPs) are pseudotyped with a homologous or heterologous envelope protein such as VSV-G. In some embodiments, the Cas12 protein is fused to a gag protein (e.g., MLVgag) via a cleavable linker, wherein cleavage of the linker in a target cell exposes an NLS located between the linker and the Cas12 protein. In some embodiments, the fusion protein or conjugate comprises (e.g., from 5′ to 3′) a gag protein (e.g., MLVgag), one or more NESs, a cleavable linker, one or more NLSs, and Cas12, as described in Banskota et al. Cell 185 (2): 250-265 (2022).

In some embodiments, the Cas12 protein is fused to a first dimerization domain, wherein the first dimerization domain is capable of dimerizing or heterodimerizing with a second dimerization domain fused to a membrane protein, and wherein the presence of a ligand promotes such dimerization and enriches the Cas12 protein, or a fusion protein or conjugate thereof, into the VLP, as described in Campbell et al., Molecular Therapy 27:151-163 (2019).

### Cells

In another aspect of the present disclosure, cells comprising the CRISPR-Cas12 system described herein are provided. The cells (e.g., which can be used to produce a cell-free system) can be eukaryotic or prokaryotic. Non-limiting examples of such cells include bacteria, archaea, plants, fungi, yeast, insect cells, and mammalian cells, such as Lactobacillus, Lactococcus, Bacillus (e.g., Bacillus subtilis), Escherichia (e.g., Escherichia coli), Clostridium, Saccharomyces or Pichia (e.g., Saccharomyces cerevisiae or Pichia pastoris), Kluyveromyces lactis, Salmonella typhimurium, Drosophila cells, Caenorhabditis elegans, human cells, and mouse cells.

In some embodiments, the cell is a prokaryotic cell, such as a bacterial cell, for example, Escherichia coli. In some embodiments, the cell is a eukaryotic cell, such as a mammalian cell or a human cell. In some embodiments, the cell is a primary eukaryotic cell, a stem cell, a tumor/cancer cell, a circulating tumor cell (CTC), or a blood cell (e.g., a T cell).

### Target Nucleic Acid or Target DNA

In some embodiments of the present disclosure, the target nucleic acid is a target DNA.

The CRISPR-Cas12 system described herein can be used to target one or more target DNA molecules, for example, target DNA molecules present in a biological sample, an environmental sample (e.g., a soil, air, or water sample), and the like.

In some embodiments, the target nucleic acid is a disease-associated gene or a gene associated with a signal-transduction biochemical pathway, or the target nucleic acid is a reporter gene. Non-limiting examples of such target nucleic acids include those listed in U.S. Provisional Patent Applications Nos. 61/736,527 and 61/748,427, filed on Dec. 12, 2012 and Jan. 2, 2013, respectively, and International Application PCT/US2013/074667, filed on Dec. 12, 2013, each of which is incorporated herein by reference in its entirety.

In the present disclosure, non-limiting examples of the target nucleic acid or target DNA include, but are not limited to:
IL1B (interleukin-1, β), XDH (xanthine dehydrogenase), TP53 (tumor protein p53), PTGIS (prostaglandin 12 (prostacyclin) synthase), MB (myoglobin), IL4 (interleukin-4), ANGPT1 (angiopoietin-1), ABCG8 (ATP-binding cassette, subfamily G (white), member 8), CTSK (cathepsin K), PTGIR (prostaglandin 12 (prostacyclin) receptor (IP)), KCNJ11 (inward rectifier potassium channel, subfamily J, member 11), INS (insulin), CRP (C-reactive protein, pentamer-associated), PDGFRB (platelet-derived growth factor receptor, β-peptide), CCNA2 (cyclin A2), PDGFB (platelet-derived growth factor β-peptide, simian sarcoma virus (v-sis)). Oncogene homologs), KCNJ5 (inward rectifier potassium channel, subfamily J, member 5), KCNN3 (potassium intermediate conductance calcium-activated channel, subfamily N, member 3), CAPN10 (capain 10), PTGES (prostaglandin E synthase), ADRA2B (adrenergic, α-2B-, receptor), ABCG5 (ATP-binding cassette, subfamily G (WHITE), member 5), PRDX2 (peroxide oxidoreductase 2), CAPN5 (capain 5), PARP14 (poly(ADP-ribose) polymerase family, member 14), MEX3C (mex-3 homolog C (Caenorhabditis elegans)), ACE angiotensin I converting enzyme (peptidyl dipeptidase A) 1), TNF (tumor necrosis factor (TNF superfamily, member 2)), IL6 (interleukin-6) (Interferon, β2)), STN (inhibin), SERPINE1 (serine protease inhibitor, clade E (microtubule connexin, plasminogen activator inhibitor type 1), member 1), ALB (albumin), ADIPOQ (adiponectin, containing C1Q and collagen domains), APOB (apolipoprotein B (including Ag (x) antigen)), APOE (apolipoprotein E), LEP (leptin) MTHFR (5,10-methylenetetrahydrofolate reductase (NADPH)), APOA1 (apolipoprotein AI), EDN1 (endothelin 1), NPPB (pro-natriuretic peptide B), NOS3 (nitric oxide synthase 3 (endothelial cells)), PPARG (peroxisome proliferator-activated receptor γ), PLAT (plasminogen activator, tissue), PTGS2 (prostaglandin intraperoxidase 2 (prostaglandin G/H synthase and cyclooxygenase)), CETP (cholesterol ester transfer protein, plasma), AGTR1 (angiotensin II receptor, type 1), HMGCR (3-hydroxy-3-methylglutaryl-CoA reductase), IGF1 (insulin-like growth factor 1 (growth hormone regulator C)), SELE (selectin E), REN (renin), PPARA (peroxisome proliferator-activated receptor a), PON1 (Phospoxanase 1), KNG1 (kininogen 1), CCL2 (Chemokine (CC motif) ligand 2), LPL (lipoprotein lipase), VWF (Von Wöhlerbrand factor), F2 (coagulation factor II (thrombin)), ICAM1 (intercellular adhesion molecule 1), TGFB1 (transforming growth factor, β1), NPPA (pro-natriuretic peptide A), IL10 (interleukin 10), EPO (erythropoietin), SOD1 (superoxide dismutase 1, soluble), VCAM1 (vascular cell adhesion molecule 1), IFNG (interferon, Y), LPA (lipoprotein, Lp (a)), MPO (myeloperoxidase), ESR1 (estrogen receptor 1), MAPK1 (mitogen-activated protein kinase 1), HP (haptoglobin), F3 (coagulation factor III) (Prothrombin kinase, tissue factor)), CST3 (cysteine protease inhibitor C), COG2 (oligomeric Golgi complex component 2), MMP9 (matrix metallopeptidase 9 (gelatinase B, 92 kDa gelatinase, 92 kDa type I V collagenase)), SERPINC1 (serine protease inhibitor peptidase inhibitor, clade C (antithrombin), member 1), F8 (coagulation factor VIII, procoagulant component), HMOX1 (Heme oxygenase (uncirculation) 1), APOC3 (apolipoprotein C-III), IL8 (interleukin 8), PROK1 (prodylin 1), CBS (cystathionine-β-synthase), NOS2 (nitric oxide synthase 2, inducible), TLR4 (Toll-like receptor 4), SELP (selectin P (granule membrane protein 140 kDa, antigen CD62)), ABCA1 (ATP-binding cassette, subfamily A (ABC1), member 1), AGT (angiotensinogen (serine protease inhibitor, clade A, member 8)), LDLR (low-density lipoprotein receptor), GPT (alanine transaminase), VEGFA (vascular endothelial growth factor A), NR3C2 (nuclear receptor subfamily 3, type C, member 2), IL18 (Interleukin-18 (interferon-γ-inducible factor)), NOS1 (nitric oxide synthase 1 (neuronal)), NR3C1 (nuclear receptor subfamily 3, group C, member 1 (glucocorticoid receptor)), FGB (fibrinogen β chain), HGF (hepatocyte growth factor (hepatocyte growth factor A; dispersing factor)), IL1A (interleukin-1, α), RETN (resistin), AKT1 (v-akt murine thymoma virus oncogene homolog 1), LIPC (lipase, liver), HSPD1 (heat shock 60 kDa protein 1 (chaperone protein)), MAPK14 (mitogen-activated protein kinase 14), SPP1 (secretory phosphoprotein 1), ITGB3 (integrin, β3 (platelet glycoprotein 111a, antigen CD61)), CAT (catalase), UTS2 (Vaporhin 2), THBD (Thrombomodulin), F10 (Coagulation Factor X), CP (Ceruloplasmin (ferrooxidase)), TNFRSF11B (Tumor Necrosis Factor Receptor Superfamily, Member 11b), EDNRA (Endothelin A Receptor), EGFR (Epidermal Growth Factor Receptor (a homolog of the erythroleukemia virus (v-erb-b) oncogene, avian)), MMP2 (Matrix Metallopeptidase 2 (Gelatinase A, 72 kDa Gelatinase, 72 kDa Type I V Collagenase)), PLG (Plexinogen), NPY (Neuropeptide Y), RHOD (ras homolog gene family, member D), MAPK8 (mitogen-activated protein kinase 8), MYC (V-Myc myeloma virus oncogene homolog (avian)), FN1 (fibronectin 1), CMA1 (chymotrypsin 1, mast cell), PLAU (plasminogen activator, urokinase), GNB3 (guanine nucleotide-binding protein (G protein), β-peptide 3), ADRB2 (adrenergic, β-2-, receptor, surface), APOA5 (apolipoprotein AV), SOD2 (superoxide dismutase 2, mitochondrial), F5 (coagulation factor V (procoagulant globulinogen, unstable factor)), VDR (vitamin D (1,25-dihydroxyvitamin D3) receptor), ALOX5 (arachidonic acid 5-lipoxygenase). HLA-DRB1 (major histocompatibility complex, class I, DRß1), PARP1 (poly(ADP-ribose) polymerase 1), CD40LG (CD40 ligand), PON2 (paraoxygenase 2), AGER (advanced glycation end products specific receptor), IRS1 (insulin receptor substrate 1), PTGS1 (prostaglandin intraperoxide synthase 1 (prostaglandin G/H synthase and cyclooxygenase)), ECEI (endothelin convertase 1), F7 (coagulation factor VII (prothrombin conversion accelerator)), URN (interleukin-1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, subfamily B (MDR/TAP), member 1), JUN (jun oncogene), IGFBP3 (insulin-like growth factor binding protein 3), CD14 (CD14 molecule), PDE5A (phosphodiesterase 5A, cGMP specific), AGTR2 (angiotensin II receptor, type 2), CD40 (CD40 molecule, TNF receptor superfamily member 5), LCATLecithin-cholesterol acyltransferase (LCT), CCR5 (Chemokine (CC motif) receptor 5), MMP1 (Matrix metallopeptidase 1 (interstitial collagenase)), TIMP1 (TIMP metallopeptidase inhibitor 1), ADM (Adrenal medullaris), DYT10 (Dystonia 10), STAT3 (Signal transduction and transcription activator 3 (acute phase response factor)), MMP3 (Matrix metallopeptidase 3 (stromallyolysin 1, progelatinase)), ELN (Elastin), USF1 (Upstream transcription factor 1), CFH (Complement factor H), HSPA4 (Heat shock 70 kDa protein 4), MMP12 (Matrix metallopeptidase 12 (macrophage elastase)), MME (Membrane metalloproteinase), F2R (Factor II (thrombin) receptor), SELL (Selectin L), CTSB (Cetase B), ANXA5 (annexin A5), ADRB1 (adrenergic, β-1-, receptor), CYBA (cytochrome b-245, α-peptide), FGA (fibrinogen α-chain), GGT1 (γ-glutamyl transpeptidase 1), LIPG (lipase, endothelial), HIF1A (hypoxia-inducible factor 1, α-subunit (basic-helical-loop-helical transcription factor)), CXCR4 (chemokine (CXC motif) receptor 4), PROC (protein C (inhibitor of coagulation factors Va and VIIIa), SCARB1 (scavenger receptor class B, member 1), CD79A (CD79a molecule, immunoglobulin-associated α), PLTP (phospholipid transfer protein), ADD1 (adductin 1 (a)), FGG (fibrinogen γ-chain), SAA1 (Serum amyloid A1), KCNH2 (voltage-gated potassium channel, subfamily H (antennae potential related), member 2), DPP4 (dipeptidyl peptidase 4), G6PD (glucose-6-phosphate dehydrogenase), NPR1 (natriuretic peptide receptor A/guanylate cyclase A (atrial natriuretic peptide receptor A)), VTN (vitamin), KIAA0101 (KIAA0101), FOS (FBJ murine osteosarcoma virus oncogene homolog), TLR2 (Toll-like receptor 2), PPIG (peptidyl prolyl isomerase G (cyclophilin G)), IL1R1 (interleukin-1 receptor, type I), AR (androgen receptor), CYP1A1 (cytochrome P450, family 1, subfamily A, polypeptide 1), SERPINA1 (serine protease inhibitor peptidase inhibitor, clade A (α-1 antiprotease, antitrypsin), member 1), MTR (5-methyltetrahydrofolate homocysteine methyltransferase), RBP4 (retinol-binding protein 4, plasma), APOA4 (apolipoprotein A-IV), CDKN2A (cyclin-dependent kinase inhibitor 2A (melanoma p16, inhibits CDK4)), FGF2 (fibroblast growth factor 2 (basic)), EDNRB Endothelin receptor type B, ITGA2 (integrin, α2 (CD49B, VLA-2 receptor α2 subunit)), CABIN1 (calcineurin-binding protein 1), SHBG (sex hormone-binding globulin), HMGB1 (high mobility group 1), HSP90B2P (heat shock protein 90 kDaß (Grp94), member 2 (pseudogene)), CYP3A4 (cytochrome P450, family 3, subfamily A, polypeptide 4), GJAl (gap junction protein, α1, 43 kDa), CAV1 (cryptin 1, cell membrane microvesicle protein, 22 kDa), ESR2 (estrogen receptor 2 (ERB)), LTA (lymphotoxin a (TNF superfamily, member 1)), GDF15 (growth differentiation factor 15), BDNF (Brain-derived neurotrophic factor), CYP2D6 (cytochrome P450, family 2, subfamily D, polypeptide 6), NGF (nerve growth factor (β polypeptide)), SP1 (Sp1 transcription factor), TGIF1 (TGFB-inducible factor homeobox 1), SRC (v-src sarcoma (Schmidt-Ruppin A-2) viral oncogene homolog (avian)), EGF (epidermal growth factor (β-Gastrosuppressin), PIK3CG (phosphoinositol-3-kinase, catalytic, γ-peptide), HLA-A (major histocompatibility complex, class I, A), KCNQ1 (voltage-gated potassium channel, KQT-like subfamily, member 1), CNR1 (cannabinoid receptor 1 (brain)), FBN1 (microfibril 1), CHKA (choline kinase), BEST1 (vitiligoma 1), APP (amyloid β (A4) precursor protein), CTNNB1 (catenin (cadherin-associated protein), β1, 88 kDa), IL2 (interleukin-2), CD36 (CD36 molecule (thrombin-sensitive protein receptor)), PRKAB1 (protein kinase, AMP-activated, β1 non-catalytic subunit), TPO (thyroid peroxidase), ALDH7A1 (Aldehyde dehydrogenase 7 family, member A1), CX3CR1 (chemokine (C-X3-C motif) receptor 1), TH (tyrosine hydroxylase), F9 (coagulation factor IX), GHI (growth hormone 1), TF (transferrin), HFE (hemochromatosis), IL17A (interleukin 17A), PTEN (phosphatase and tensin homolog), GSTMI (glutathione S-transferase μl), DMD (muscular dystrophy protein), GATA4 (GATA-binding protein 4), F13A1 (coagulation factor XIII, A1 polypeptide), TTR (transthyretin), FABP4 (fatty acid-binding protein 4, adipocytes), PON3 (paraoxyphosphophosphatase 3), APOC1 (apolipoprotein C1), INSR (insulin receptor), TNFRSF1B (Tumor necrosis factor receptor superfamily, member 1B), HTR2A (5-hydroxytryptamine (serotonin) receptor 2A), CSF3 (colony-stimulating factor 3 (granulocytes)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony-stimulating factor 2 (granulocytes) Macrophages), KDR (kinase insertion domain receptor receptor (type I II receptor tyrosine kinase)), PLA2G2A (phospholipase A2, type IIA (platelets, synovial fluid)), B2M (β-2-microglobulin), THBS1 (thrombin-sensitive protein 1), GCG (glucagon), RHOA (ras homolog gene family, member A), ALDH2 (aldehyde dehydrogenase 2 family (mitochondria)), TCF7L2 (transcription factor 7-like 2 (T cell-specific HMG box)), BDKRB2 (bradykinin receptor B2), NFE2L2 (erythrocyte-derived nuclear factor 2-like protein), NOTCH1 (Notch homolog 1, translocation-related (Drosophila)), UGT1A1 (UDP glucuronyl transferase 1 family, polypeptide A1), IFNA1 (interferon, α1), PPARD (Peroxisome proliferator-activated receptor 8), SIRT1 (longevity protein (silencing mating type I signaling regulator 2 homolog) 1 (Saccharomyces cerevisiae)), GNRH1 (gonadotropin-releasing hormone 1 (luteinizing hormone-releasing hormone)), PAPPA (pregnancy-associated plasma protein A, pilocarpine 1), ARR3 (inhibitor protein 3, retinal (X-inhibitor protein)), NPPC (natriuretic peptide precursor C), AHSP (alpha-hemoglobin stabilizer), PTK2 (PTK2 protein tyrosine kinase 2), IL13 (interleukin-13), MTOR (rapamycin mechanical target (serine/threonine kinase)), ITGB2 (integrin, B2 (complement component 3 receptor 3 and 4 subunits)), GSTT1 (glutathione S-transferase 01), IL6ST (interleukin-6 signaling factor (gp130)). The following are listed: (tumor suppressor M receptor), CPB2 (carboxypeptidase B2 (plasma)), CYP1A2 (cytochrome P450, family 1, subfamily A, polypeptide 2), HNF4A (hepatocyte nuclear factor 4, a), SLC6A4 (solute carrier family 6 (neurotransmitter transporter, serotonin), member 4), PLA2G6 (phospholipase A2, type VI (cytosol-dependent, calcium-dependent)). TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldolol reductase family 1, member A1 (aldolol reductase)), ALDH9A1 (aldol dehydrogenase family 9, member A1), BGLAP (bone γ-carboxyglutamate (GLA) protein), MTTP (microsomal triglyceride transfer protein), MTRR (5-methyltetrahydrofolate-homocysteine methyltransferase reductase), SULTIA3 (sulfotransferase family, cytosolic, 1A, phenol preferred, member 3), RAGE (renal tumor antigen), C4B (complement component 4B (Chito blood type), P2RY12 (purinergic receptor P2Y, G-protein coupled, 12), RNLS (renin, FAD-dependent amine oxidase), CREBI (cAMP response element-binding protein 1), POMC (pro-melanocortin), RAC1 (ras-associated C3 botulinum toxin substrate 1 (rho family, small GTP-binding protein Rac1)), LMNA (nuclear lamin NC), CD59 (CD59 molecule, complement regulatory protein), SCN5A (sodium channel, voltage-gated, V-type, α-subunit), CYP1B1 (cytochrome P450, family 1, subfamily B, polypeptide 1), MIF (macrophage migration inhibitor (glycosylation inhibitor)), MMP13 (matrix metallopeptidase 13 (collagenase 3)), TIMP2 (TIMP metallopeptidase inhibitor 2), CYP19A1 (cytochrome P450, family 19, subfamily A) The following peptides are involved: polypeptide 1, CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonin deficient)), SELPLG (selectin P ligand), AOC3 (amine oxidase, copper-containing 3 (vascular adhesion protein 1)), and CTSLI (cathepsins).L1), PCNA (proliferating cell nuclear antigen), IGF2 (insulin-like growth factor 2 (auxin regulator A)), ITGB1 (integrin, β1 (fibronectin receptor, β polypeptide, antigen CD29 including MDF2, MSK12)), CAST (calpain inhibitor), CXCL12 (chemokine (CXC motif) ligand 12 (stromal cell-derived factor 1)), IGHE (immunoglobulin homeostasis ¿), KCNE1 (voltage-gated potassium channel, Isk-related family, member 1), TFRC (transferrin receptor (p90, CD71)), COLIA1 (collagen, type I, α1), COL1A2 (collagen, type I, α2), IL2RB (interleukin-2 receptor, B), PLA2G10 (phospholipase A2, type X), ANGPT2 (Angiopoietin 2), PROCR (protein C receptor, endothelial (EPCR)), NOX4 (NADPH oxidase 4), HAMP (hepaxicillin antimicrobial peptide), PTPN11 (protein tyrosine phosphatase, non-receptor type 1), SLC2A1 (solute carrier family 2 (facilitated glucose transporter), member 1), IL2RA (interleukin 2 receptor, a), CCL5 (chemokine (CC motif) ligand 5), IRF1 (interferon regulator 1), CFLAR (CASP8 and FADD-like apoptosis regulator), CALCA (calcitonin-associated peptide a), EIF4E (eukaryotic translation initiation factor 4E), GSTP1 (glutathione S-transferase pil), JAK2 (Janus kinase 2), CYP3A5 (cytochrome P450, family 3, subfamily A, peptide 5). HSPG2 (heparin sulfate proteoglycan 2), CCL3 (chemokine (CC motif) ligand 3), MYD88 (myeloid differentiation primary response gene (88)), VIP (vasoactive intestinal peptide), SOAT1 (sterol O-acyltransferase 1), ADRBKI (adrenergic, β, receptor kinase 1), NR4A2 (nuclear receptor subfamily 4, type A, member 2), MMP8 (matrix metallopeptidase 8 (neutrophil collagenase)), NPR2 (natriuretic peptide receptor B/guanylate cyclase B (atrial natriuretic peptide receptor B)), GCHI (GTP cyclase 1), EPRS (glutamyl-prolyl-tRNA synthetase), PPARGCIA (peroxisome proliferator-activated receptor γ, coactivator 1α), F12 (coagulation factor XII (Hagmann factor)), PECAM1 (platelet/endothelial cell adhesion molecule), CCL4 (chemokine (CC motif) ligand 4), SERPINA3 (serine protease inhibitor peptidase inhibitor, clade A (α-1 antiprotease, antitrypsin), member 3), CASR (calcocyte receptor), GJA5 (gap junction protein, α5 (40 kDa), FABP2 (fatty acid-binding protein 2, intestinal), TTF2 (transcription termination factor, RNA polymerase II), PROS1 (protein S (a)), CTF1 (cardiac nutrient 1), SGCB (inotropic protein, β (43 kDa dystrophin-associated glycoprotein)), YMEIL1 (YME1-like 1 (Saccharomyces cerevisiae)), CAMP (calciferine antimicrobial peptide), ZC3H12A (zinc-finger CCCH type 12A), AKRIB1 (aldolol reductase family 1, member B1 (aldolose reductase)), DES (desmin), MMP7 (matrix metallopeptidase 7 (matrixolytic factor, uterus)), AHR (aromatic hydrocarbon receptor), CSF1 (colony-stimulating factor 1 (macrophages)), HDAC9 (histone deacetylase 9), CTGF (Connective tissue growth factor), KCNMA1 (high-conductivity calcium-activated potassium channel, subfamily M, a member 1), UGT1A (UDP-glucuronyl transferase 1 family, polypeptide A complex locus), PRKCA (protein kinase C, a), COMT (catechol-β-methyltransferase), S100B (S100 calcium-binding protein B), EGRI (early growth response protein 1), PRL (prolactin) IL15 (Interleukin-15), DRD4 (Dopamine receptor D4), CAMK2G (calcium-calmodulin-dependent protein kinase Ily), SLC22A2 (Solute carrier family 22 (organic cation transporter), member 2), CCL11 (Chemokine (CC motif) ligand 11), PGF (B321 placental growth factor), THPO (thrombopoietin), GP6 (glycoprotein VI (platelet)), TACRI (tachykinin receptor 1), NTS (neurotensin-lowering peptide), HNF1A (HNF1 homeobox A), SST (somatostatin), KCNDI (voltage-gated potassium channel, Shal-related subfamily, member 1), LOC646627 (phospholipase inhibitor), TBXAS1 (thromboxane A synthase 1 (platelet)), CYP2J2 (Cytochrome P450, Family 2, Subfamily J, Polypeptide 2), TBXA2R (Thromboxane A2 receptor), ADHIC (Alcohol dehydrogenase 1C (Class I), γ-peptide), ALOX12 (Arachidonicate 12-lipoxygenase), AHSG (α-2-HS-glycoprotein), BHMT (Betaine homocysteine methyltransferase), GJA4 (Gap junction protein, α4, 37 kDa), SLC25A4 (Solute carrier family 25 (mitochondrial carrier; adenine nucleotide transporter), Member 4), ACLY (ATP citrate lyase), ALOX5AP (Arachidonicate 5-lipoxygenase-activating protein), NUMA1 (Nuclear mitogen protein 1), CYP27B1 (Cytochrome P450, Family 27, Subfamily B, Polypeptide 1), CYSLTR2 Cysteine leukotriene receptor 2, SOD3 (superoxide dismutase 3, extracellular), LTC4S (leukotriene C4 synthase), UCN (urocortin), GHRL (gastrin/obesity-inhibiting precursor peptide), APOC2 (apolipoprotein C-II), CLEC4A (C-type lectin domain family 4, member A), KBTBD10 (Kelch repeat and BTB (POZ) domain-included protein), TNC (tenosynovin C), TYMS (thymidine synthase). SHCI (SHC (containing Src homology 2 domain) converting protein 1), LRP1 (low-density lipoprotein receptor-associated protein 1), SOCS3 (cytokine signaling inhibitor 3), ADHIB (alcohol dehydrogenase 1B (class I), β-peptide), KLK3 (kallikrein-associated peptidase 3), HSD11B1 (hydroxysterol (11-β) dehydrogenase 1), VKORCI (vitamin K epoxide reductase complex, subunit 1), SERPINB2 (serine protease inhibitor peptidase inhibitor, clade B (ovalbumin), member 2), TNS1 (tensin 1), RNF19A (circular finger protein 9A), EPOR (erythropoietin receptor), ITGAM (integrin, aM (complement component 3 receptor subunit 3)), PITX2 (pair-like homology domain 2), MAPK7 Mitogen-activated protein kinase 7 (MIRV), FCGR3A (Fc fragment of IgG, low affinity 111a, receptor (CD16a)), LEPR (leptin receptor), ENG (endothelial glycoprotein), GPX1 (glutathione peroxidase 1), GOT2 (aspartate aminotransferase 2, mitochondrial (aspartate aminotransferase 2)), HRH1 (histamine receptor H1), NR112 (nuclear receptor subfamily 1, type I, member 2), CRH (corticotropic hormone-releasing hormone), HTR1A (5-hydroxytryptamine (serotonin) receptor 1A), VDAC1 (voltage-dependent anion channel 1), HPSE (heparinoid enzyme), SFTPD (surfactant protein D), TAP2 (transporter 2, ATP-binding cassette, subfamily B (MDR/TAP)), RNF123 (Cyclic finger protein 123), PTK2B (PTK2B protein tyrosine kinase 2B), NTRK2 (neurotrophic tyrosine kinase, receptor, type 2), IL6R (interleukin-6 receptor), ACHE (acetylcholinesterase (Yt blood type)), GLPIR (glucagon-like peptide-1 receptor), GHR (growth hormone receptor), GSR (glutathione reductase), NQO1 (NAD (P) H dehydrogenase, quinone) 1) NR5A1 (nuclear receptor subfamily 5, type A, member 1), GJB2 (gap junction protein, β2, 26 kDa), SLC9A1 (solute carrier family 9 (sodium/hydrogen exchanger), member 1), MAOA (monoamine oxidase A), PCSK9 (proprotein convertase subtilisin/kexin9 type), FCGR2A (Fc fragment of IgG, low affinity IIa, receptor (CD32)), SERPINF1 (serine protease inhibitor, peptidase inhibitor, clade F (α-2 antifibrinolytic enzyme, pigment epithelium-derived factor), member 1), EDN3 (endothelin 3), DHFR (dihydrofolate reductase), GAS6 (growth arrest-specific protein 6), SMPD1 (sphingomyelin phosphodiesterase 1, acid lysosome), UCP2 (uncoupling protein 2) (Mitochondrial, proton carrier), TFAP2A (transcription factor AP-2a (activator enhancer-binding protein 2a)), C4BPA (complement component 4-binding protein, a), SERPINF2 (serine protease inhibitor peptidase inhibitor, clade F (α-2 anti-fibrinolytic enzyme, pigment epithelium-derived factor), member 2), TYMP (thymidine phosphorylase), ALPP (alkaline phosphatase, placental (Regan isoenzyme)), CXCR2 (chemokine (CXC motif) receptor 2), SLC39A3 (solute carrier family 39 (zinc transporter), member 3), ABCG2 (ATP-binding cassette, subfamily G (WHITE), member 2), ADA (adenosine deaminase), JAK3 (Janus kinase 3), HSPA1A (heat shock 70 kDa protein 1A), FASN (fatty acid synthase), FGF1 (Fibroblast growth factor 1 (acidic)), F11 (coagulation factor XI), ATP7A (ATPase, Cu++ transporter, α-peptide), CR1 (complement component (3b/4b) receptor 1 (Knops blood group)), GFAP (glial fibrillary acidic protein), ROCK1 (Rho-associated, containing coiled-coil protein kinase 1), MECP2 (methyl CpG-binding protein 2 (Rett syndrome)), MYLK (myosin light chain kinase).BCHE (butyrylcholinesterase), LIPE (lipase, hormone-sensitive), PRDX5 (peroxide oxidoreductase 5), ADORA1 (adenosine A1 receptor), WRN (Werner syndrome, RecQ helicase-like), CXCR3 (chemokine (CXC motif) receptor 3), CD81 (CD81 molecule), SMAD7 (SMAD family member 7), LAMC2 (laminusoids, γ2), MAP3K5 (mitogen-activated protein kinase kinase 5), CHGA (chromogranin A (parathyroid secretory protein 1)), IAPP (pancreatic amyloid polypeptide), RHO (rhodopsin), ENPP1 (exonucleotide pyrophosphatase/phosphodiesterase 1), PTHLH (parathyroid hormone-like hormone), NRG1 (neuroregulatory protein 1), VEGFC (vascular endothelial growth factor C), ENPEP (Glutamyl aminopeptidase (aminopeptidase A)), CEBPB (CCAAT/enhancer-binding protein (C/EBP), B), NAGLU (N-acetylglucosidase, α-), F2RL3 (coagulation factor II (thrombin) receptor-like 3), CX3CL1 (chemokine (C-X3-C motif) ligand 1), BDKRB1 (bradykinin receptor B1), ADAMTS13 (ADAM metallopeptidase with thrombin-sensitive protein type 1 motif, 13), ELANE (elastase, expressed by neutrophils), ENPP2 (exonucleotide pyrophosphatase/phosphodiesterase 2), CISH (cytokine-induced SH2-containing protein), GAST (gastrin), MYOC (myofibrillarin, trabecular meshwork can induce glucocorticoid responses), ATP1A2 (ATPase, Na+/K+ transporter, α2 polypeptide), NF1 (neurofibroma protein 1), GJB1 (gap junction protein, β1, 32 kDa), MEF2A (myotrophic factor 2A), VCL (neoprotein), BMPR2 (bone morphogenetic protein receptor, type II (serine/threonine kinase)), TUBB (microtubule protein, B), CDC42 (cell cycle 42 (GTP-binding protein, 25 kDa)), KRT18 (keratin 18), HSF1 (heat shock transcription factor) 1) MYB (v-myb myeloblastoma virus oncogene homolog (avian)), PRKAA2 (protein kinase, AMP-activated, α2 catalytic subunit), ROCK2 (Rho-associated coiled-coil protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), 81), CTH (cystathionine enzyme (cystathionine γ-lyase)), CTSS (cathepsins S), VAV2 (vav2 guanylate exchange factor), NPY2R (neuropeptide Y receptor Y2), IGFBP2 (insulin-like growth factor binding protein 2, 36 kDa), CD28 (CD28 molecule), GSTA1 (glutathione S-transferase al). PPIA (peptidyl prolyl isomerase A (cyclophilin A)), APOH (apolipoprotein H (β-2-glycoprotein I)), S100A8 (S100 calcium-binding protein A8), IL11 (interleukin 11), ALOX15 (arachidonic acid 15-lipoxygenase), FBLNI (peronein 1), NR1H3 (nuclear receptor subfamily 1, type H, member 3), SCD (stearoyl-CoA desaturase (A-9-desaturase)), GIP (gastric inhibitory polypeptide), CHGB (chromogranin B (secretory granulin 1)), PRKCB (protein kinase C, B), SRD5A1 (steroid-5-α reductase a polypeptide 1 (3-oxo-5α-steroid 84-dehydrogenase α1)). HSD11B2 (hydroxysterol (11-β) dehydrogenase 2), CALCRL (calcitonin receptor-like), GALNT (UDP-N-acetyl-α-D-galactosamine: polypeptide N-acetylgalactosamine transferase 2 (GalNAc-T2)), ANGPTL4 (angiopoietin-like 4), KCNN4 (potassium intermediate/low conductance calcium-activated channel, subfamily N, member 4)), PIK3C2A (phosphatidylinositol-3-kinase, class 2, α-peptide), HBEGF (heparin-binding EGF-like growth factor), CYP7A1 (cytochrome P450, family 7, subfamily A, peptide 1), HLA-DRB5 (major histocompatibility complex, class I I, DRB5), BNIP3 (BCL2/adenovirus E1B19 kDa interacting protein 3), GCKR (glucosokinase (hexokinase 4) regulatory protein), S100A12 (S100 calcium-binding protein A12), PADI4 (peptidyl arginine deiminase, type I V), HSPA14 (heat shock 70 kDa protein 14), CXCR1 (chemokine (CXC motif) receptor 1), H19 (H19, maternally imprinted transcript (non-protein encoded)), KRTAP19-3 (Keratin-associated protein 19-3), IDDM2 (insulin-dependent diabetes mellitus 2), RAC2 (ras-associated C3 botulinum toxin substrate 2 (rho family, small GTP-binding protein Rac2)), RYRI (renine base receptor 1 (skeletal)), CLOCK (clock homolog (mouse)), NGFR (nerve growth factor receptor (TNFR superfamily, member 16)), DBH (dopamine β-hydroxylase (dopamine β-monooxygenase)), CHRNA4 (cholinergic receptor, nicotinic, α4), CACNA1C (calcium channel, voltage-dependent, L-type, α1C subunit), PRKAG2 (protein kinase, AMP-activated, γ2 non-catalytic subunit), CHAT (choline acetyltransferase), PTGDS (prostaglandin D2 synthase 21 kDa (brain)), NR1H2 (nuclear receptor subfamily 1, type H) Members 2), TEK (TEK tyrosine kinase, endothelial), VEGFB (vascular endothelial growth factor B), MEF2C (myotrophic factor 2C), MAPKAPK2 (mitogen-activated protein kinase 2), TNFRSF11A (tumor necrosis factor receptor superfamily, member 11a, NFKB activator), HSPA9 (heat shock 70 kDa protein 9 (lethal protein)), CYSLTRICysteine leukotriene receptor 1, MAT1A (methionine adenosine transferase I, a), OPRL1 (opioid receptor-like 1), IMPA1 (inositol (muscle)-1 (or 4)-monosophosphatase 1), CLCN2 (chloride channel 2), DLD (dihydrolipoamide dehydrogenase), PSMA6 (proteasome (precursor, macroprotein factor) subunit, a type, 6), PSMB8 (proteasome (precursor, macroprotein factor) subunit, β type, 8 (large multifunctional peptidase 7)), CHI3L1 (chitosanase 3-like 1 (chondroglycoprotein-39)), ALDHIB1 (aldehyde dehydrogenase 1 family, member B1), PARP2 (poly(ADP-ribose) polymerase 2), STAR (steroidogenic acute-phase regulatory protein), LBP (lipopolysaccharide-binding protein). ABCC6 (ATP-binding cassette, subfamily C (CFTR/MRP), member 6), RGS2 (G protein signaling regulator 2, 24 kDa), EFNB2 (hepatin-B2), GJB6 (gap connexin, B6, 30 kDa), APOA2 (apolipoprotein A-II), AMPDI (adenosine monophosphate deaminase 1), DYSF (dysferlin, limb-girdle muscular dystrophy 2B (autosomal recessive)), FDFT1 (farniyl diphosphate farniyltransferase 1), EDN2 (endothelin 2), CCR6 (chemokine (CC motif) receptor 6), GJB3 (gap connexin, β3, 31 kDa), IL1RL1 (interleukin-1 receptor-like 1), ENTPD1 (Exonucleotide triphosphate diphosphate hydrolase 1), BBS4 (Bardet-Biedl syndrome 4), CELSR2 (cadherin, EGFLAG heptakin G receptor 2 (flamingo homolog, Drosophila)), F11R (F11 receptor), RAPGEF3 (Rap guanylate exchange factor (GEF) 3), HYAL1 (hyaluronic acid glucosaminease 1), ZNF259 (zinc finger protein 259), ATOX1 (ATX1 antioxidant protein 1 homolog (yeast)), ATF6 (activating transcription factor 6), KHK (hexyl kinase (fructokinase)), SATI (spermine/spermine N1-acetyltransferase 1), GGH (gamma-glutamyl hydrolase (conjugating enzyme, folic acid polygamma-glutamyl hydrolase)), TIMP4 (TIMP metallopeptidase inhibitor 4), SLC4A4 (solute carrier family 4, sodium bicarbonate cotransporter, member 4), PDE2A (phosphodiesterase 2A, cGMP stimulated), PDE3B (phosphodiesterase 3B, cGMP inhibited), FADS1 (fatty acid desaturase 1), FADS2 (fatty acid desaturase 2), TMSB4X (thymosin β4, X-linked), TXNIP (thioredoxin interacting protein), LIMS1 (LIM and senescent cell antigen-like domain 1). RHOB (ras homolog gene family, member B), LY96 (lymphocyte antigen 96), FOXO1 (forkhead box 01), PNPLA2 (containing the Patatin-like phospholipase domain 2), TRH (thyrotropin-releasing hormone), GJC1 (gap connexin, γ1, 45 kDa), SLC17A5 (solute carrier family 17 (anion/glycan transporter), member 5), FTO (fat mass and obesity-related), GJD2 (gap connexin, 82, 36 kDa), PSRC1 (proline/serine-rich coil-and-coil protein 1), CASP12 (caspases 12 (gene/pseudogene)), GPBARI (G protein-coupled bile acid receptor 1), PXK (containing the PX domain serine/threonine kinase), IL33 (interleukin 33), TRIB1 (Tribbles homolog 1 (Drosophila), PBX4 (pre-B-cell leukemia homeobox 4), NUPR1 (nuclear protein, transcription regulator 1), 15-Sep (15 kDa selenoprotein), CILP2 (middle cartilage protein 2), TERC (telomerase RNA component), GGT2 (γ-glutamyl transpeptidase 2).MT-CO1 (mitochondria encoding cytochrome c oxidase I) or UOX (uric acid oxidase, pseudogene).

Genes associated with trinucleotide repeat amplification disorders, non-restrictive examples include AR (androgen receptor), FMR1 (fragile x intellectual disability 1), HTT (huntin), DMPK (myotonic dystrophy protein kinase), FXN (mitochondrial ataxia protein), ATXN2 (spinocerebellar ataxia protein 2), ATNI (atrophic protein 1), FEN1 (fragment structure-specific endonuclease 1), TNRC6A (trinucleotide repeat containing 6A), PABPN1 (poly(A)-binding protein, nucleus 1), JPH3 (affinity protein 3), MED15 (mediator complex subunit 15), ATXN1 (spinocerebellar ataxia protein 1), ATXN3 (spinocerebellar ataxia protein 3), TBP (TATA box-binding protein), CACNAIA (calcium channel, voltage-dependent, P/Q type, alA subunit), ATXN80S (ATXN8 anti-strand (non-protein coding)), PPP2R2B (protein phosphatase 2, regulatory subunit B, β), ATXN7 (spinocerebellar ataxia protein 7), TNRC6B (trinucleotide repeat containing 6B), TNRC6C (trinucleotide repeat containing 6C), CELF3 (CUGBP, Elav-like family member 3), MAB21L1 (mab-21-like 1 (C. elegans)), MSH2 (MutS homolog 2, colon cancer, polyposis-free type 1 (E. coli)), TMEM185A (transmembrane protein 185A), SIX5 (SIX homeobox 5), CNPY3 (canopy 3 homolog (zebrafish)), FRAXE (fragile site, folate type, rare type, fra(X)(q28) E), GNB2 (guanine nucleotide-binding protein (G protein), β-peptide 2), RPL14 (ribosomal protein L14), ATXN8 (spinocerebellar ataxia protein 8), INSR (insulin receptor), TTR (transthyretin), EP400 (E1A-binding protein p400), GIGYF2 (GRB10-interacting GYF protein 2), OGG1 (8-oxoguanine DNA glycosylase), STC1 (Calcium 1), CNDP1 (Carnosine dipeptidase 1 (metallopeptidase M20 family)), C10orf2 (chromosome 10 open reading frame 2), MAML3 (Drosophila melanogaster), DKC1 (congenital dyskeratosis 1, dyskeratin), PAXIP1 (PAX interacting protein 1 with transcription activation domain), CASK (calcium/calmodulin-dependent serine protein kinase (MAGUK family), MAPT (microtubule-associated protein tau), SP1 (Sp1 transcription factor), POLG (polymerase (DNA-directed), γ), AFF2 (AF4/FMR2 family, member 2), THBS1 (thrombin-sensitive protein 1), TP53 (tumor protein p53), ESR1 (estrogen receptor 1), CGGBP1 (CGG triplet repeat binding protein 1), ABT1 (basic transcription activator 1). KLK3 (kallikrein-associated peptidase 3), PRNP (prion protein), JUN (jun oncogene), KCNN3 (potassium intermediate/low-conductivity calcium-activated channel, subfamily N, member 3), BAX (BCL2-associated X protein), FRAXA (fragile site, folate type, rare type, fra (X) (q27.3) A (giant testis, intellectual disability)), KBTBD10 (Kelch repeat and BTB (POZ) domain-included protein 10), MBNL1 (blind muscle-like (Drosophila)), RAD51 (RAD51 homolog (RecA homolog, E. coli) (Saccharomyces cerevisiae)), NCOA3 (nuclear receptor coactivator 3), ERDA1 (extended repeat domain, CAG/CTG1), TSCI (tuberous sclerosis 1), COMP (chondrocyte oligomeric matrix protein), GCLC (Glutamine cysteine ligase, catalytic subunit), RRAD (Ras-associated diabetes), MSH3 (mutS homolog 3 (E. coli)), DRD2 (dopamine receptor D2), CD44 (CD44 molecule (Indian blood type)), CTCF (CCCTC binding factor (zinc finger protein)), CCNDI (cyclin D1), CLSPN (clonoxin homolog (Xenopus laevis)). MEF2A (Myocyte Enhancer Factor 2A), PTPRU (Protein Tyrosine Phosphatase, Receptor Type, U), GAPDH (Glyceraldehyde-3-Phosphate Dehydrogenase), TRIM22 (Trimosome Protein 22), WT1 (Wilmesoma 1), AHR (Aromatic Hydrocarbon Receptor), GPX1 (Glutathione Peroxidase 1), TPMT (Thiopurine Methyltransferase), NDP (Nori's Disease (Pseudoglioma)), ARX (Angiotensin-Free Related Homeobox), MUS81 (MUS81 Endonuclease Homolog (Saccharomyces Sacchariformis)), TYR (Tyrosinase (Oculocutaneous Albinism IA)), EGRI (Early Growth Response Protein 1), UNG (Uracil DNA Glycosylase), NUMBL (Numbness Homolog (Drosophila)-like), FABP2 (Fatty Acid Binding Protein 2, Intestine), EN2 (Zergoid Homeobox 2), CRYGC (Lens protein, γC), SRP14 (signal recognition particle 14 kDa (homologous AluRNA binding protein)), CRYGB (Lens protein, YB), PDCD1 (programmed cell death 1), HOXA1 (homeobox A1), ATXN2L (spinocerebellar ataxia protein 2-like), PMS2 (PMS2 post-meiotic segregation of 2-like proteins (Saccharomyces cerevisiae)), GLA (galactosidase, a), CBL (Cas-Br-M (mouse) tropical retrovirus transformation sequence), FTHI (ferritin, heavy polypeptide 1), IL12RB2 (interleukin-12 receptor, B2), OTX2 (orthodentate homeobox 2), HOXA5 (homeobox A5), POLG2 (polymerase (DNA-directed), γ2, helper subunit), DLX2 (Terminal reduced homeobox 2), SIRPA (signal regulatory protein a), OTX1 (orthodontic homeobox 1), AHRR (aromatic hydrocarbon receptor antagonist), MANF (midbrain astrocyte-derived neurotrophic factor), TMEM158 (transmembrane protein 158 (gene/pseudogene)) or ENSG00000078687.

MD-related genes, including but not limited to: (ABCA4) ATP-binding cassette, subfamily A (ABC1), member 4; ACHM1 achromatopsia (rod monochromatopsia) 1; ApoE, apolipoprotein E (ApoE); C1QTNF5 (CTRP5), Clq and tumor necrosis factor-associated protein 5 (C1QTNF5); C2 complement, complement 2 (C2); C3 complement, complement (C3); CCL2, chemokine (CC motif) ligand 2 (CCL2); CCR2, chemokine (CC motif) receptor 2 (CCR2); CD36 differentiation antigen cluster 36; CFB, complement receptor B; CFH, complement factor CFHH; CFHR1, complement factor H-related 1; CFHR3, complement factor H-related 3; CNGB3 cyclic nucleotide-gated channel β3. C-plasmin (CP), CRP, C-reactive protein (CRP), CST3 cysteine protease inhibitor C or cysteine protease inhibitor 3 (CST3), CTSD, cathepsin D (CTSD), CX3CR1, chemokine (C-X3-C motif) receptor 1, ELOVL4, ultra-long chain fatty acid extension 4, ERCC6, excision repair of cross-complement rodent repair defects, complementation group 6, FBLN5, anti-aging protein-5, FBLN5, anti-aging protein 5, FBLN6, anti-aging protein 6, FSCN2 bundle protein (FSCN2), HMCN1, hemicontin 1, HMCN1, hemicontin 1, HTRA1, HtrA serine peptidase 1 (HTRA1), HTRA1, HtrA serine peptidase 1, IL-6, interleukin-6, IL-8 Interleukin-8, LOC387715, putative protein, LEKHA1, platelet-containing leukocyte C kinase substrate homology domain family A member 1 (PLEKHA1), PROMIProminin 1 (PROM1 or CD133), PRPH2, peripheral protein-2RPGR GTPase modulator, SERPING1, serine protease inhibitor, peptidase inhibitor, clade G, member 1 (C1-inhibitor), TCOF1, molasses TIMP3 metalloproteinase inhibitor 3 (TIMP3) or TLR3 Toll-like receptor 3.

Examples of reporter genes include, but are not limited to, glutathione S-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments of the present disclosure, the target nucleic acid is the TTR (transthyretin) gene. Transthyretin familial amyloid polyneuropathy (transthyretin familial amyloid polyneuropathy, TTR-FAP) is a rare autosomal dominant, multisystem disease primarily characterized by peripheral neuropathy, and is caused by pathogenic variants in the TTR gene encoding transthyretin. Editing the TTR gene using the CRISPR-Cas12 system described in the present disclosure can be used to treat transthyretin familial amyloid polyneuropathy. In some embodiments, the guide sequence of the guide polynucleotide is SEQ ID NO: 14.

In some embodiments of the present disclosure, the target nucleic acid is the HBB (hemoglobin beta) gene. Sickle cell anemia and beta-thalassemia are hereditary anemias caused by mutations in the HBB gene encoding the adult hemoglobin beta subunit. Editing the HBB gene using the CRISPR-Cas12 system described in the present disclosure can be used to treat diseases such as sickle cell anemia and beta-thalassemia. In some embodiments, the guide sequence of the guide polynucleotide is SEQ ID NO: 15.

In some embodiments of the present disclosure, the target nucleic acid is the HBG (hemoglobin gamma-globin) gene. Clinical studies have found that activating fetal HBG expression in patients with thalassemia and achieving a higher level of HbF can alleviate symptoms of thalassemia patients, and even completely cure the disease. Editing the HBG gene using the CRISPR-Cas12 system described in the present disclosure can be used to treat diseases such as thalassemia. In some embodiments, the guide sequence of the guide polynucleotide is SEQ ID NO: 16.

In some embodiments of the present disclosure, the target nucleic acid is a disease- or disorder-associated gene. In some embodiments of the present disclosure, the target nucleic acid is a disease-associated gene. In some embodiments of the present disclosure, the disease-associated gene is a pathogenic gene that directly causes the disease. In some embodiments of the present disclosure, the disease-associated gene is an abnormal gene that directly causes the disease, or a gene whose expression is abnormal. For example, the gene has an unfavorable mutation, thereby leading to occurrence of the disease. As another example, the gene is overexpressed or underexpressed, thereby leading to occurrence of the disease.

In some embodiments of the present disclosure, the disease or disorder is a hematologic disease or disorder, an ophthalmic disease or disorder, a neurological disease or disorder, a respiratory disease or disorder, a liver disease or disorder, a metabolic disease or disorder, a cancer, or an infectious disease.

In some embodiments of the present disclosure, the disease or disorder is selected from: hemophilia A, Best vitelliform macular dystrophy, B-cell acute lymphoblastic leukemia, hemophilia B, CDKL5 deficiency disorder, CLN2 disease, Niemann-Pick disease type C, Dravet syndrome, FOXGI syndrome, GM1 gangliosidosis, GM2 gangliosidosis, HIV infection, HSV infection, Usher syndrome type IB, Usher syndrome type IIA, mucopolysaccharidosis type IIIA, mucopolysaccharidosis type IIIB, Gaucher disease type III, mucopolysaccharidosis type II, type II diabetes, mucopolysaccharidosis type IV, Gaucher disease type I, mucopolysaccharidosis type I, type I diabetes, Usher syndrome type I, KCNQ2 epileptic encephalopathy, Leber hereditary optic neuropathy, Leigh syndrome, Prader-Willi syndrome, SLC13A5 deficiency, X-linked myotubular myopathy, X-linked retinoschisis, X-linked retinitis pigmentosa, alpha-1 antitrypsin deficiency, alpha-mannosidosis, alpha-thalassemia, beta-thalassemia, Alzheimer's disease, Bardet-Biedl syndrome, retinitis Punctata albescens, leukocyte adhesion deficiency type I, galactosemia, bladder cancer, overactive bladder, phenylketonuria, nasopharyngeal carcinoma, Bietti crystalline dystrophy, pyruvate kinase deficiency, erectile dysfunction, autosomal recessive congenital ichthyosis, adult polyglucosan body disease, post-traumatic arthritis, homozygous familial hypercholesterolemia, fragile X syndrome, thalassemia, hypophosphatasia, epilepsy, multiple myeloma, multiple system atrophy, frontotemporal dementia, catecholaminergic polymorphic ventricular tachycardia, Fabry disease, Fanconi anemia, aromatic L-amino acid decarboxylase deficiency, radiation-induced xerostomia, non-Hodgkin lymphoma, non-muscle-invasive bladder cancer, non-alcoholic fatty liver disease, non-small cell lung cancer, hypertrophic cardiomyopathy, hypertrophic scar, obesity, Charcot-Marie-Tooth disease type 1A, Charcot-Marie-Tooth disease type 2A, pulmonary hypertension, Friedreich's ataxia, peritoneal cancer, liver cancer, hepatocellular carcinoma, dry age-related macular degeneration, Sjogren's syndrome, hyperuricemia, hyperlipidemia, Gaucher disease, autism spectrum disorder, osteoarthritis, bone marrow failure syndrome, citrullinemia type I, coronary heart disease, cystinosis, melanoma, Huntington's disease, amyotrophic lateral sclerosis, urgency urinary incontinence, acute intermittent porphyria, acute lymphoblastic leukemia, spinocerebellar ataxia, spinal muscular atrophy with respiratory distress type 1, spinal muscular atrophy, familial amaurotic dementia, methylmalonic acidemia, thyroid cancer, Duchenne muscular dystrophy, anaplastic astrocytoma, intermittent claudication, junctional epidermolysis bullosa, glioma, glioblastoma, corneal graft rejection, colorectal cancer, progressive multifocal leukoencephalopathy, progressive familial intrahepatic cholestasis, giant axonal neuropathy, Canavan disease, cocaine addiction, Krabbe disease, Crigler-Najjar syndrome, oral cancer, Angelman syndrome, diffuse intrinsic pontine glioma, Lafora disease, rheumatoid arthritis, sickle cell disease, lymphedema, ovarian cancer, chronic lymphocytic leukemia, chronic granulomatous disease, anemia of chronic kidney disease, chronic pain, chronic hepatitis B, Menkes disease, cystic fibrosis, Netherton syndrome, ornithine transcarbamylase deficiency, Parkinson's disease, Pompe disease, uveitis, prostate cancer, vestibular schwannoma, myotonic dystrophy, ankylosing spondylitis, castration-resistant prostate cancer, glaucoma, achromatopsia, ischemic heart failure, lysosomal storage disease, sarcoma, breast cancer, Rett syndrome, triple-negative breast cancer, Sandhoff disease, color blindness, heart failure with reduced ejection fraction, neuronal ceroid lipofuscinosis, adrenoleukodystrophy, renal cell carcinoma, wet age-related macular degeneration, eczema, thrombocytopenia with immunodeficiency syndrome, esophageal cancer, optic neuropathy, optic atrophy, retinal vein occlusion, retinitis pigmentosa, rhodopsin-mediated autosomal dominant retinitis pigmentosa, ependymoma, fallopian tube cancer, bilateral vestibulopathy, Stargardt disease, diabetic macular edema, diabetic neuropathy, diabetic retinopathy, diabetic peripheral neuropathic pain, diabetic foot, glycogen storage disease, glycogen storage disease type Ia, glycogen storage disease type IIb, atopic dermatitis, hearing loss, hearing impairment, head and neck cancer, head and neck squamous cell carcinoma, Wilson disease, stable angina pectoris, Usher syndrome, choroideremia, congenital amaurosis, congenital adrenal hyperplasia, cardiomyopathy, angina pectoris, heart failure, novel coronavirus infection, pleural mesothelioma, acne vulgaris, severe combined immunodeficiency, critical limb ischemia, oculopharyngeal muscular dystrophy, pancreatic cancer, graft-versus-host disease, inherited retinal dystrophy, hereditary angioedema, hepatitis B, metachromatic leukodystrophy, psoriatic arthritis, recessive dystrophic epidermolysis bullosa, infantile malignant osteopetrosis, dystrophic epidermolysis bullosa, scleroderma, primary immunodeficiency, heterozygous familial hypercholesterolemia, limb-girdle muscular dystrophy type 2B, limb-girdle muscular dystrophy type 2C, limb-girdle muscular dystrophy type 2D, limb-girdle muscular dystrophy type 2E, limb-girdle muscular dystrophy type 21, limb-girdle muscular dystrophy type 2L, limb ischemic disease, lipoprotein lipase deficiency, severe congenital neutropenia, wrinkles, stroke, sciatica, schizophrenia, depression, drug addiction, autism, idiopathic pulmonary fibrosis, transthyretin (ATTR) amyloidosis, AATD liver disease, and AATD lung disease.

The genes associated with transthyretin (ATTR) amyloidosis include, but are not limited to, ATTR;

The genes associated with Leber hereditary optic neuropathy include, but are not limited to, MT-ND4;

The genes associated with AATD liver disease include, but are not limited to, AATD; The genes associated with AATD lung disease include, but are not limited to, AATD;

The genes associated with graft-versus-host disease include, but are not limited to, the thymidine kinase gene;

The genes associated with inherited retinal dystrophy include, but are not limited to, RPE65;

The genes associated with spinal muscular atrophy include, but are not limited to, SMN1;

The genes associated with osteoarthritis include, but are not limited to, TGF-β1;

The genes associated with hemophilia A include, but are not limited to, factor VIII;

The genes associated with hemophilia B include, but are not limited to, factor IX;

The genes associated with cystic fibrosis include, but are not limited to, CFTR;

The genes associated with Parkinson's disease include, but are not limited to, Gad1, Gad2, PTBP1, and REST;

The genes associated with Usher syndrome include, but are not limited to, USH2A;

The genes associated with alpha-thalassemia, beta-thalassemia, and sickle cell disease include, but are not limited to, BCL11A, HBG, HBA, and HBB;

The genes associated with pulmonary hypertension include, but are not limited to, eNOS;

The genes associated with Stargardt disease include, but are not limited to, ABCA4;

The genes associated with age-related macular degeneration include, but are not limited to, VEGFA and VEGFR;

The genes associated with glaucoma include, but are not limited to, AQP1;

The genes associated with idiopathic pulmonary fibrosis include, but are not limited to, CTGF;

The genes associated with Alzheimer's disease include, but are not limited to, NGF;

The genes associated with coronary heart disease include, but are not limited to, VEGFA and bFGF;

The genes associated with anemia of chronic kidney disease include, but are not limited to, EPO;

The genes associated with congenital amaurosis include, but are not limited to, RPE65;

The genes associated with retinitis pigmentosa include, but are not limited to, PDE6B;

The genes associated with phenylketonuria include, but are not limited to, PAH; The genes associated with epilepsy include, but are not limited to, GAT1.

### Therapeutic Applications

In another aspect of the present disclosure, a pharmaceutical composition comprising a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, or a cell described herein is provided. The pharmaceutical composition can comprise, for example, an AAV vector encoding the Cas12 protein or Cas12 protein mutant described herein and the guide polynucleotide described herein. The pharmaceutical composition can comprise, for example, lipid nanoparticles comprising the guide polynucleotide described herein and mRNA encoding the Cas12 protein. The pharmaceutical composition can comprise, for example, a lentiviral vector comprising the guide polynucleotide described herein and mRNA encoding the Cas12 protein. The pharmaceutical composition can comprise, for example, a virus-like particle comprising the guide polynucleotide described herein and the Cas12 protein, or a ribonucleoprotein complex formed by the guide polynucleotide and the Cas12 protein.

In another aspect of the present disclosure, the present disclosure relates to the use of a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, a cell described herein, a pharmaceutical composition described herein, or a kit described herein for cleaving or editing a target nucleic acid in a mammalian cell.

In another aspect of the present disclosure, the present disclosure relates to the use of a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, a cell described herein, a pharmaceutical composition described herein, or a kit described herein for any one of the following: cleaving one or more target nucleic acid molecules or creating a nick in one or more target nucleic acid molecules; activating or upregulating expression of one or more target nucleic acid molecules; activating or inhibiting transcription of one or more target nucleic acid molecules; inactivating one or more target nucleic acid molecules; visualizing, labeling, or detecting one or more target nucleic acid molecules; binding one or more target nucleic acid molecules; transporting one or more target nucleic acid molecules; and masking one or more target nucleic acid molecules.

In another aspect of the present disclosure, the present disclosure relates to the use of a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, a cell described herein, a pharmaceutical composition described herein, or a kit described herein for modifying one or more target nucleic acid molecules, wherein modifying one or more target nucleic acid molecules comprises one or more of: nucleic acid base substitution, nucleic acid base deletion, nucleic acid base insertion, cleavage of a target nucleic acid, nucleic acid methylation, and nucleic acid demethylation.

In another aspect of the present disclosure, the present disclosure relates to the use of a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, a cell described herein, a pharmaceutical composition described herein, or a kit described herein in diagnosing, treating, or preventing a disease or disorder associated with a target nucleic acid.

In another aspect of the present disclosure, the present disclosure relates to the use of a Cas12 protein described herein, a Cas12 protein mutant described herein, a guide polynucleotide described herein, a Cas12 inactivated variant described herein, a Cas12 fusion protein or conjugate described herein, a nucleic acid described herein, a CRISPR-Cas12 system described herein, a vector system described herein, a delivery system described herein, a cell described herein, a pharmaceutical composition described herein, or a kit described herein in the manufacture of a medicament for diagnosing, treating, or preventing a disease or disorder associated with a target nucleic acid.

In some embodiments, the pharmaceutical composition is delivered in vivo to a human subject. The pharmaceutical composition can be delivered via any effective route. Exemplary routes of administration include, but are not limited to, intravenous infusion, intravenous injection, intraperitoneal injection, intramuscular injection, intratumoral injection, subcutaneous injection, intradermal injection, intraventricular injection, intravascular injection, intracerebellar injection, intraocular injection, subretinal injection, intravitreal injection, intracameral injection, intratympanic injection, intranasal administration, and inhalation.

### Diagnostic Applications

In another aspect of the present disclosure, an in vitro composition comprising the CRISPR-Cas12 system described herein and a labeled detector DNA that cannot hybridize to the guide polynucleotide described herein is provided.

In another aspect of the present disclosure, the present disclosure relates to the use of the CRISPR-Cas12 system described herein for detecting a target nucleic acid in a nucleic acid sample suspected of containing the target nucleic acid.

In some embodiments, a method of detecting a target DNA comprises a Cas12 protein fused to a fluorescent protein or another detectable label and a guide polynucleotide comprising a guide sequence specific for the target DNA. Binding of Cas12 to the target DNA can be visualized by microscopy or other imaging methods.

In some embodiments, a method of detecting a target nucleic acid in a cell-free system results in the generation of a detectable label or an enzymatic activity. For example, by using a Cas12 protein, a guide polynucleotide comprising a guide sequence specific for the target nucleic acid, and a detectable label, the target nucleic acid is recognized by Cas12. Binding of Cas12 to the target nucleic acid triggers its DNase activity, which results in cleavage of the target nucleic acid and the detectable label.

In some embodiments, the detectable label is a DNA linked to a fluorescent probe and a quencher. The intact detectable DNA links the fluorescent probe and the quencher, thereby quenching fluorescence. After the detectable DNA is cleaved by Cas12, the fluorescent probe is released from the quencher and exhibits fluorescent activity. This method can be used to determine whether a target DNA is present in a lysed cell sample, a lysed tissue sample, a blood sample, a saliva sample, an environmental sample (e.g., a water, soil, or air sample), or another lysed cellular or cell-free sample. This method can also be used to detect a pathogen, such as a virus or a bacterium, or to diagnose a disease state, such as cancer.

In some embodiments, the detection of target nucleic acids helps diagnose diseases and/or pathological conditions, or the presence of viral or bacterial infections.

### EXAMPLES

The present disclosure is further illustrated below by way of examples, but the disclosure is not limited to the scope of the examples described herein. Experimental methods in the following examples that do not specify specific conditions were performed according to conventional methods and conditions, or as selected according to the product instructions.

### Example 1: Construction of pCDH-CMV-EGFP-Reporter3-EF1a-Puro Cell Line

1. Construction of the Lentiviral Expression Plasmid pCDH-CMV-EGFP-Reporter3-EFla-Puro for the GFP Reporter System

Synthesize a GFP fragment (SEQ ID NO: 2) containing the detection system:

tctagagcgagaaaagccttgtttgccaccatggaacggctcggagatca
tcattgcgtcgcgaggtgagcaagggcgaggagctgttcaccggggtggt
gcccatcctggtcgagctggacggcgacgtaaacggccacaagttcagcg
tgtccggcgaggggagggcgatgccacctacggcaagctgaccctgaagt
tcatctgcaccaccggcaagctgcccgtgccctggcccaccctcgtgacc
accctgacctacggcgtgcagtgcttcagccgctaccccgaccacatgaa
gcagcacgacttcttcaagtccgccatgcccgaaggctacgtccaggagc
gcaccatcttcttcaaggacgacggcaactacaagacccgcgccgaggtg
aagttcgagggcgacaccctggtgaaccgcatcgagctgaagggcatcga
cttcaaggaggacggcaacatcctggggcacaagctggagtacaactaca
acagccacaacgtctatatcatggccgacaagcagaagaacggcatcaag
gtgaacttcaagatccgccacaacategaggacggcagegtgcagctcgc
cgaccactaccagcagaacacccccatcggcgacggccccgtgctgctgc
ccgacaaccactacctgagcacccagtccaagctgagcaaagaccccaac
gagaagcgcgatcacatggtcctgctggagttcgtgaccgccgccgggat
cactctcggcatggacgagctgtacaagtaagcggccgc

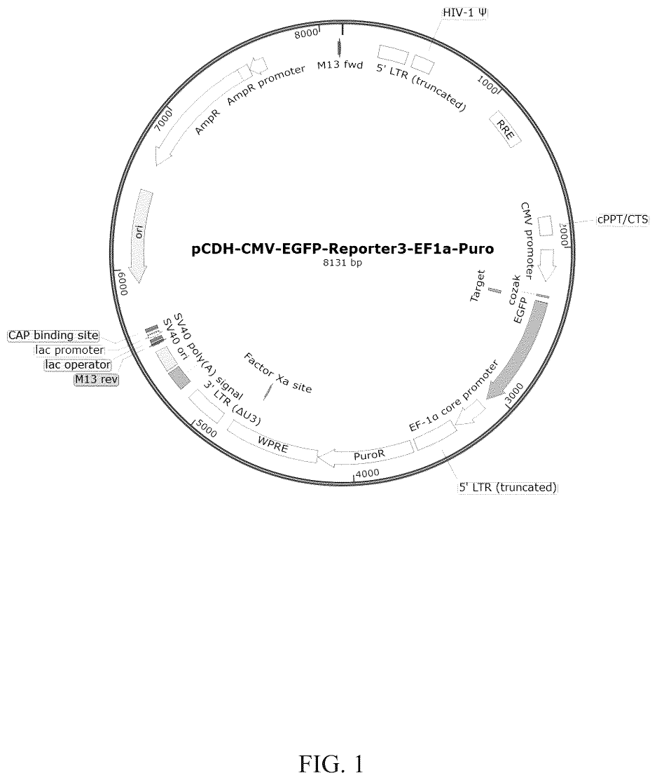

The GFP fragment was digested with XbaI+NotI to obtain the digested product, which was then ligated with the XbaI+NotI digested product of pCDH-CMV-MCS-EF1-Puro plasmid (Ubibio) using T4 DNA ligase (Thermo Scientific). The ligation was then performed on Stb13 cells, and after overnight incubation at 37° C. on ampicillin-resistant plates, clones were picked and sequenced to obtain the pCDH-CMV-EGFP-Reporter3-EF1a-Puro plasmid (SEQ ID NO: 3), the pattern of which is shown in FIG. 1.

The specific principle of the reporter system is as follows: In the unedited reporter system, there is a 32 bp base insertion between the start codon (bold black text) and the normal reading frame of GFP, which interrupts the normal reading frame of GFP (underlined part), and GFP is not expressed. Using CRISPR-Cas technology, the gRNA target site is set inside GFP (the framed part is the target sequence corresponding to sgRNA). By editing and generating indels, there is a chance to restore the normal reading frame of GFP, so that GFP can be expressed normally. The higher the Cas editing efficiency, the higher the rate of indels that restore the correct GFP reading frame. The editing efficiency of the Cas protein is characterized by the number of cells that can express GFP normally by flow cytometry.

### 2. Lentiviral Packaging

The pCDH-CMV-EGFP-Reporter3-EF1a-Puro plasmid identified by sequencing was mixed with viral packaging helper plasmids pMD2.G (Miaoling Biotechnology) and psPAX2 (Miaoling Biotechnology) in a 1:1:1 molar ratio and then transfected into 293T cells using PEI. After 48 hours, the culture supernatant was collected and filtered through a 0.45 μm filter to obtain the crude pCDH-CMV-EGFP-Reporter3-EF1a-Puro virus.

3. Construction and Detection of 293T Cell Lines Infected with Virus

293T cells were infected with pCDH-CMV-EGFP-Reporter3-EF1a-Puro crude virus. After 48 hours of infection, the medium was changed, and 2 μg/ml of puromycin was added for selection. The selected cells were then subjected to limiting dilution for monoclonal selection, and the selected monoclonal cells were used as the detection cell line (referred to as the Reporter3 cell line).

### Example 2: Cas12i Mutant Design and Vector Construction, and Report System Editing Efficiency Testing

### 1. Determination of Mutation Sites

The wild-type Cas12i protein (SEQ ID NO: 1 in CN111757889B) was selected for mutation. The amino acid sequence of the wild-type protein is shown below (1045 aa).

SEQ ID NO: 1:
MKKVEVSRPYQSLLLPNHRKFKYLDETWNAYKSVKSLLHRFLVCAYGAVP
FNKFVEVVEKVDNDQLVLAFAVRLFRLVPVESTSFAKVDKANLAKSLANH
LPVGTAIPANVQSYFDSNFDPKKYMWIDCAWEADRLAREMGLSASQFSEY
ATTMLWEDWLPLNKDDVNGWGSVSGLFGEGKKEDRQQKVKMLNNLLNGIK
KNPPKDYTQYLKILLNAFDAKSHKEAVKNYKGDSTGRTASYLSEKSGEIT
ELMLEQLMSNIQRDIGDKQKEISLPKKDVVKKYLESESGVPYDQNLWSQA
YRNAASSIKKTDTRNFNSTLEKFKNEVELRGLLSEGDDVEILRSKFFSSE
FHKTPDKFVIKPEHIGFNNKYNVVAELYKLKAEATDFESAFATVKDEFEE
KGIKHPIKNILEYIWNNEVPVEKWGRVARFNQSEEKLLRIKANPTVECNQ
GMTFGNSAMVGEVLRSNYVSKKGALVSGEHGGRLIGQNNMIWLEMRLLNK
GKWETHHVPTHNMKFFEEVHAYNPSLADSVNVRNRLYRSEDYTQLPSSIT
DGLKGNPKAKLLKRQHCALNNMTANVLNPKLSFTINKKNDDYTVIIVHSV
EVSKPRREVLVGDYLVGMDQNQTASNTYAVMQVVKPKSTDAIPFRNMWVR
FVESGSIESRTLNSRGEYVDQLNHDGVDLFEIGDTEWVDSARKFFNKLGV
KHKDGTLVDLSTAPRKAYAFNNFYFKTMLNHLRSNEVDLTLLRNEILRVA
NGRFSPMRLGSLSWTTLKALGSFKSLVLSYFDRLGAKEMVDKEAKDKSLF
DLLVAINNKRSNKREERTSRIASSLMTVAQKYKVDNAVVHVVVEGNLSST
DRSASKAHNRNTMDWCSRAVVKKLEDMCNLYGFNIKGVPAFYTSHQDPLV
HRADYDDPKPALRCRYSSYSRADFSKWGQNALAAVVRWASNKKSNTCYKV
GAVEFLKQHGLFADKKLTVEQFLSKVKDEEILIPRRGGRVFLTTHRLLAE
STFVYLNGVKYHSCNADEVAAVNICLNDWVIPCKKKMKEESSASG

The three-dimensional structure of the Cas12i protein (SEQ ID NO: 1) was predicted and simulated using bioinformatics analysis and AI techniques. The possible DNA binding, recognition, and cleavage sites of Cas12i were analyzed in conjunction with the three-dimensional structure analysis. Mutant clones were constructed using molecular cloning point mutagenesis methods targeting these sites.

TABLE 1
Designed Cas12i mutants (Round 1 design)

| Mutation site | Name |
|---|---|
| W170R | Cas12i-05 |
| S174R | Cas12i-06 |
| E225R | Cas12i-07 |
| N229R | Cas12i-08 |
| D233R | Cas12i-09 |
| T235R | Cas12i-10 |
| Y241R | Cas12i-11 |
| S246R | Cas12i-12 |
| M253R | Cas12i-13 |
| Q256R | Cas12i-14 |
| S259R | Cas12i-15 |
| N260R | Cas12i-16 |
| Q294R | Cas12i-17 |
| N295R | Cas12i-18 |
| I549R | Cas12i-19 |
| T550R | Cas12i-20 |
| D551R | Cas12i-21 |
| E658R | Cas12i-22 |
| L662R | Cas12i-23 |
| N663R | Cas12i-24 |
| S664R | Cas12i-25 |
| Y668R | Cas12i-26 |
| D678R | Cas12i-27 |
| F680R | Cas12i-28 |

### 2. Construction of Mutant Clones

After identifying the specific mutation site, the mutated base is introduced using primers to construct expression clones containing different mutation sites. The following explanation uses the construction of the N260R mutant clone as an example.

First, primers were designed based on the mutation site N260R to construct the mutant cloning plasmid Cas12i-pCDNA3.1-16, which can be used to express the mutant Cas12i-16 and gRNA. The specific primer sequences are shown in Table 2.

TABLE 2
Primer sequences used to construct the Cas12i mutant clone Cas12i-pCDNA3.1-16

| Primer name | Primer sequence |
|---|---|
| Cas12-PF1 | CGTTTAAACTTAAGCTTACCGGTGCCAC (SEQ ID NO: 4) |
| Cas12-PR1 | TGTGCATAGTCACACCGTCGCGAG (SEQ ID NO: 5) |
| Cas12i-16-PF1 | CTGATGAGCagaATTCAGAGAGACATCGGCGACAAG (SEQ ID NO: 6) |
| | (Underlined text indicates the introduction of an amino acid mutation) |
| Cas12i-16-PR1 | CTCTGAATtctGCTCATCAGCTGCTCCAGCATC (SEQ ID NO: 7) |
| | (Underlined text indicates the introduction of an amino acid mutation) |

Primers were designed to target the N260R mutation site, and the mutation site was introduced using primers Cas12I-16-PF1 and Cas12I-16-PR1. Using the synthesized plasmid pXC12-68-GFPgRNA (SEQ ID NO: 8) encoding the wild-type protein as a template, PCR amplification was performed using Cas12i-16-PF1+Cas12-PR1 (EasyBio, UltraHiPF™ DNA Polymerase Kit) to obtain the fragment Cas12i-16-F1. PCR amplification was performed using Cas12i-16-PR1 and Cas12-PF1 to obtain the fragment Cas12i-16-F2. The wild-type plasmid pXC12-68-GFPgRNA was digested with HindIII+KpnI, and the 5646 bp fragment was recovered by gel electrophoresis. It was then recombined in vitro with fragments Cas12i-16-F1 and Cas12i-16-F2 (NEB, E2611L, Gibson Assembly® Master Mix). The resulting mutant clone plasmid Cas12i-pCDNA3.1-16 (SEQ ID NO: 9) was obtained by heat shock transformation of E. coli.

The same method was used to construct mutant clonal plasmids expressing other mutants and the same gRNA. The only difference between these and the Cas12i-pCDNA3.1-16 plasmids described above is the mutation site of the expressed mutant.

### 3. Detect the Editing Efficiency of Mutants in the Reporting System.

Plating: When the pCDH-CMV-EGFP-Reporter3-EFla-Puro cell line (Reporter3 cell line) reaches a confluence of 70-80%, it is plated into 24-well plates with a cell number of 5×10\hat{ }5 cells/well.

Transfection: Transfection was performed 12-14 h after plating. 100 μl Opti-MEM, 1.5 μl PEI (Yisheng Bio, Polyethylenimine Linear (PEI) MW25000), and 500 ng of mutant clone plasmid were added to each well of a 24-well plate. After mixing and incubation at room temperature for 20 minutes, the mixture was added to the Reporter3 cell line for transfection. The culture medium was replaced with fresh medium overnight, and the cells were cultured for 72 h. Flow cytometry was used to detect the editing efficiency of different mutant clones based on the proportion of GFP-positive cells. The ratio of GFP-positive cells edited by different mutants to the proportion of GFP-positive cells edited by wild-type Cas12i was calculated. The results are shown in Table 3.

TABLE 3
Results of the first round of editing efficiency testing

| | | Folds of editing |
|---|---|---|
| | | efficiency compared |
| Mutation | | to wild-type Cas12i |
| site | Mutant | control |
| W170R | Cas12i-05 | 0.32 |
| S174R | Cas12i-06 | 0.31 |
| E225R | Cas12i-07 | 0.71 |
| N229R | Cas12i-08 | 1.06 |
| D233R | Cas12i-09 | 1.62 |
| T235R | Cas12i-10 | 1.64 |
| Y241R | Cas12i-11 | 0.42 |
| S246R | Cas12i-12 | 1.07 |
| M253R | Cas12i-13 | 1.21 |
| Q256R | Cas12i-14 | 1.48 |
| S259R | Cas12i-15 | 1.49 |
| N260R | Cas12i-16 | 2.31 |
| Q294R | Cas12i-17 | 0.72 |
| N295R | Cas12i-18 | 2.13 |
| I549R | Cas12i-19 | 0.97 |
| T550R | Cas12i-20 | 1.12 |
| D551R | Cas12i-21 | 0.93 |
| E658R | Cas12i-22 | 0.99 |
| L662R | Cas12i-23 | 0.99 |
| N663R | Cas12i-24 | 0.61 |
| S664R | Cas12i-25 | 0.84 |
| Y668R | Cas12i-26 | 1.10 |
| D678R | Cas12i-27 | 1.06 |
| F680R | Cas12i-28 | 1.15 |
| E681R | Cas12i-29 | 0.84 |
| N/A | Wild-type Cas12i | 1.00 |
| | control | |
| N/A | NC-PEI | 0.08 |
| N/A | NC | 0.02 |

In Table 3, the NC blank control is the Reporter3 cell line that has not been transfected with the mutant clone plasmid, and the NC-PEI blank control is the Reporter3 cell line that has not been transfected with the mutant clone plasmid but has been supplemented with PEI. Cas12i-16 and Cas12i-18 were selected in the first round of screening for subsequent combined mutations.

Based on the results of the first round of mutations, the entire three-dimensional structure is corrected and labeled, and the data from the first round is placed in a new model for predictive analysis. Finally, possible mutation sites in the second round are analyzed and predicted, and mutations and detections are performed. This process is repeated, and through multiple rounds of mutation, selection, and accumulation, the optimal mutation combination for Cas12i is determined.

Eight rounds of mutations were performed, and the mutants from rounds 2 to 8 are shown in Tables 4-9. Mutant clonal plasmids were constructed using the same method described above, and editing efficiency was detected in a reporter system. The ratio of the proportion of GFP-positive cells edited with different mutants to the proportion of GFP-positive cells edited with wild-type Cas12i or specific mutants was calculated, and the results are shown in Tables 4-9. The NC blank control is the Reporter3 cell line not transfected with the mutant clonal plasmid, and the NC-PEI blank control is the Reporter3 cell line not transfected with the mutant clonal plasmid but with added PEI.

TABLE 4
Results of the Second Round of Editing Efficiency Testing

| | | | | Folds of editing |
|---|---|---|---|---|
| | | | | efficiency |
| | | | | compared to |
| | | | | wild-type |
| Mutation site | | | Mutant | Cas12i control |
| N295R | N260R | | Cas12i-30 | 1.84 |
| N295R | N260R | D233R | Cas12i-31 | 1.34 |
| N295R | N260R | Q11R | Cas12i-33 | 1.56 |
| N295R | N260R | D166R | Cas12i-34 | 1.80 |
| N295R | N260R | N168R | Cas12i-35 | 1.48 |
| N295R | N260R | T313R | Cas12i-36 | Not detected |
| N295R | N260R | N325R | Cas12i-39 | 1.80 |
| N295R | N260R | N369R | Cas12i-40 | 1.74 |
| N295R | N260R | N443R | Cas12i-41 | 1.21 |
| N295R | N260R | Q450R | Cas12i-42 | Not detected |
| N295R | N260R | N456R | Cas12i-43 | 1.63 |
| N295R | N260R | E601R | Cas12i-44 | 1.59 |
| N295R | N260R | P605R | Cas12i-45 | 1.71 |
| N295R | N260R | D678E | Cas12i-46 | 1.62 |
| N295R | N260R | K872R | Cas12i-47 | 1.66 |
| N295R | N260R | E875R | Cas12i-48 | 1.83 |
| N295R | N260R | N879R | Cas12i-50 | 1.74 |
| N295R | N260R | N884R | Cas12i-51 | 1.76 |
| N295R | N260R | K872R + | Cas12i-52 | 1.15 |
| | | E875R + | | |
| | | D876R + | | |
| | | N879R + | | |
| | | N884R | | |
| N295R | N260R | D166R + N168R | Cas12i-54 | 1.80 |
| N295R | N260R | T313R + | Cas12i-55 | Not detected |
| | | N317R + N325R | | |
| N/A | N/A | N/A | Wild-type | 1.00 |
| | | | Cas12i control | |
| N/A | N/A | N/A | NC | 0.03 |
| N/A | N/A | N/A | NC-PEI | 0.09 |

TABLE 5
Results of the third round of editing efficiency testing

| | | | | Folds of editing |
|---|---|---|---|---|
| | | | | efficiency compared |
| | | | | to wild-type |
| mutation site | | | mutant | Cas12i control |
| N295R | N260R | T235R | Cas12i-32 | 1.42 |
| N295R | N260R | N317R | Cas12i-37 | 1.87 |
| N295R | N260R | E321R | Cas12i-38 | 1.96 |
| N295R | N260R | D876R | Cas12i-49 | 1.26 |
| N295R | N260R | S306R | Cas12i-56 | Not detected |
| N295R | N260R | K310R | Cas12i-57 | Not detected |
| N295R | N260R | T354R | Cas12i-58 | 1.26 |
| N295R | N260R | P355R | Cas12i-59 | 1.74 |
| N295R | N260R | D356R | Cas12i-60 | 1.37 |
| N295R | N260R | V359R | Cas12i-61 | 1.79 |
| N295R | N260R | I407R | Cas12i-62 | Not detected |
| N295R | N260R | N409R | Cas12i-63 | 1.53 |
| N295R | N260R | V446R | Cas12i-64 | 2.04 |
| N295R | N260R | C448R | Cas12i-65 | Not detected |
| N295R | N260R | H702R | Cas12i-66 | 1.84 |
| N295R | N260R | K703R | Cas12i-67 | 1.70 |
| N295R | N260R | D704R | Cas12i-68 | 1.52 |
| N295R | N260R | G705R | Cas12i-69 | 2.09 |
| N295R | N260R | L778R | Cas12i-70 | 1.61 |
| N295R | N260R | D782R | Cas12i-71 | 1.61 |
| N295R | N260R | K787R | Cas12i-72 | 1.75 |
| N295R | N260R | E788R | Cas12i-73 | 2.30 |
| N295R | N260R | M789R | Cas12i-74 | Not detected |
| N295R | N260R | V790R | Cas12i-75 | 1.68 |
| N295R | N260R | V804R | Cas12i-76 | 1.90 |
| N295R | N260R | N807R | Cas12i-77 | 1.86 |
| N295R | N260R | S811R | Cas12i-78 | 2.00 |
| N295R | N260R | E815R | Cas12i-79 | 1.96 |
| N295R | N260R | M863R | Cas12i-80 | 1.09 |
| N295R | N260R | A869R | Cas12i-81 | 1.92 |
| N/A | N/A | N/A | Wild-type | 1.00 |
| | | | Cas12i | |
| | | | Comparison | |
| N/A | N/A | N/A | NC | 0.06 |
| N/A | N/A | N/A | NC-PEI | 0.07 |

In the third round of screening, Cas12i-69 was selected for subsequent combined mutations.

TABLE 6
Results of the fourth and fifth rounds of editing efficiency testing

| | | | | | Folds of editing |
|---|---|---|---|---|---|
| | | | | | efficiency |
| | | | | | compared to |
| | | | | | wild-type |
| mutation site | | | | mutant | Cas12i control |
| N295R | N260R | G705R | T850E | Cas12i-102 | 1.38 |
| N295R | N260R | G705R | T850R | Cas12i-103 | 1.59 |
| N295R | N260R | G705R | S849E | Cas12i-104 | 1.84 |
| N295R | N260R | G705R | S849R | Cas12i-105 | 2.13 |
| N295R | N260R | G705R | A857E | Cas12i-106 | 1.81 |
| N295R | N260R | G705R | A857R | Cas12i-107 | 2.21 |
| N295R | N260R | G705R | V61E | Cas12i-108 | 1.93 |
| N295R | N260R | G705R | L332E | Cas12i-110 | 2.02 |
| N295R | N260R | G705R | L332R | Cas12i-111 | 2.29 |
| N295R | N260R | G705R | Q262E | Cas12i-112 | 2.15 |
| N295R | N260R | G705R | Q262R | Cas12i-113 | 1.70 |
| N295R | N260R | G705R | I249E | Cas12i-114 | 1.47 |
| N295R | N260R | G705R | I249R | Cas12i-115 | 1.51 |
| N295R | N260R | G705R | I249F | Cas12i-116 | 1.56 |
| N295R | N260R | G705R | L526E | Cas12i-117 | 1.91 |
| N295R | N260R | G705R | L526R | Cas12i-118 | 1.88 |
| N295R | N260R | G705R | N449R | Cas12i-122 | 1.75 |
| N295R | N260R | G705R | K926E | Cas12i-123 | 1.64 |
| N295R | N260R | G705R | Q929E | Cas12i-125 | 1.76 |
| N295R | N260R | G705R | Q929R | Cas12i-126 | 1.85 |
| N295R | N260R | G705R | N930R | Cas12i-128 | 2.05 |
| N295R | N260R | G705R | A933E | Cas12i-129 | 2.09 |
| N295R | N260R | G705R | A933R | Cas12i-130 | 1.81 |
| N295R | N260R | G705R | F962E | Cas12i-131 | 2.07 |
| N295R | N260R | G705R | F962R | Cas12i-132 | 2.02 |
| N295R | N260R | G705R | Q971E | Cas12i-133 | 2.15 |
| N295R | N260R | G705R | Q971R | Cas12i-134 | 2.22 |
| N295R | N260R | G705R | L475R | Cas12i-135 | 1.94 |
| N295R | N260R | G705R | V469R | Cas12i-136 | 1.91 |
| N295R | N260R | G705R | L438R | Cas12i-137 | 1.84 |
| N295R | N260R | G705R | A794R | Cas12i-138 | 2.04 |
| N295R | N260R | G705R | L553R | Cas12i-139 | 1.71 |
| N295R | N260R | G705R | C567R | Cas12i-140 | 2.15 |
| N295R | N260R | G705R | V58R | Cas12i-143 | 1.98 |
| N295R | N260R | G705R | Q11E | Cas12i-83 | 1.84 |
| N295R | N260R | G705R | T313E | Cas12i-85 | 1.51 |
| N295R | N260R | G705R | N317E | Cas12i-86 | 1.44 |
| N295R | N260R | G705R | N325E | Cas12i-87 | 1.98 |
| N295R | N260R | G705R | N369E | Cas12i-88 | 1.75 |
| N295R | N260R | G705R | N443E | Cas12i-89 | 1.46 |
| N295R | N260R | G705R | Q450E | Cas12i-90 | 1.33 |
| N295R | N260R | G705R | K872E | Cas12i-93 | 1.99 |
| N295R | N260R | G705R | N879E | Cas12i-94 | 2.03 |
| N295R | N260R | G705R | N884E | Cas12i-95 | 1.91 |
| N295R | N260R | G705R | P355E | Cas12i-96 | 2.17 |
| N295R | N260R | G705R | T354E | Cas12i-97 | 1.50 |
| N295R | N260R | G705R | N409E | Cas12i-98 | 1.91 |
| N295R | N260R | G705R | D590R | Cas12i-99 | 2.11 |
| N/A | N/A | N/A | N/A | Wild-type Cas12i | 1.00 |
| | | | | control | |
| N/A | N/A | N/A | N/A | NC-PEI | 0.04 |
| N/A | N/A | N/A | N/A | NC | 0.03 |

TABLE 7
Results of the Sixth Round of Editing Efficiency Testing

| | | | | | Folds of editing |
|---|---|---|---|---|---|
| | | | | | efficiency |
| | | | | | compared to |
| mutation site | | | | mutant | Cas12i-69 |
| N295R | N260R | G705R | R606K | Cas12i-144 | 0.90 |
| N295R | N260R | G705R | M618Y | Cas12i-145 | Not detected |
| N295R | N260R | G705R | Q632E | Cas12i-146 | 0.82 |
| N295R | N260R | G705R | F644Y | Cas12i-147 | 0.54 |
| N295R | N260R | G705R | G845Δ | Cas12i-148 | 0.71 |
| N295R | N260R | G705R | N846D | Cas12i-149 | 0.69 |
| N295R | N260R | G705R | R860S | Cas12i-150 | 0.69 |
| N295R | N260R | G705R | C866L | Cas12i-151 | Not detected |
| N295R | N260R | G705R | A869G | Cas12i-152 | 0.85 |
| N295R | N260R | G705R | K872N | Cas12i-153 | 0.94 |
| N295R | N260R | G705R | Y881H | Cas12i-154 | 0.92 |
| N295R | N260R | G705R | I1031K | Cas12i-155 | Not detected |
| N295R | N260R | G705R | N/A | Cas12i-69 | 1.00 |
| N/A | N/A | N/A | N/A | NC-PEI | 0.02 |
| N/A | N/A | N/A | N/A | NC | 0.01 |

TABLE 8
Results of the Seventh Round of Editing Efficiency Testing

| | | | | | Folds of editing |
|---|---|---|---|---|---|
| | | | | | efficiency |
| | | | | | compared to |
| mutation site | | | | mutant | Cas12i-69 |
| N260R | G705R | V446R | N/A | Cas12i-156 | 1.06 |
| N260R | G705R | E788R | N/A | Cas12i-157 | 1.24 |
| N260R | G705R | S811R | N/A | Cas12i-158 | 1.04 |
| N260R | G705R | D166R + | N/A | Cas12i-159 | 1.15 |
| | | N168R | | | |
| N295R | N260R | G705R | Q186K | Cas12i-161 | 1.13 |
| N295R | N260R | G705R | N193K | Cas12i-162 | 1.27 |
| N295R | N260R | G705R | N194K | Cas12i-163 | 1.26 |
| N295R | N260R | G705R | N197K | Cas12i-164 | 1.17 |
| N295R | N260R | G705R | E255K | Cas12i-165 | 1.31 |
| N295R | N260R | G705R | Q256K | Cas12i-166 | 1.19 |
| N295R | N260R | G705R | Q262K | Cas12i-167 | 1.20 |
| N295R | N260R | G705R | E271K | Cas12i-168 | 1.32 |
| N295R | N260R | G705R | E328K | Cas12i-169 | 1.29 |
| N295R | N260R | G705R | N416K | Cas12i-170 | 1.18 |
| N295R | N260R | G705R | E418K | Cas12i-171 | 1.28 |
| N295R | N260R | G705R | L475K | Cas12i-172 | 0.50 |
| N295R | N260R | G705R | E504K | Cas12i-173 | 1.15 |
| N295R | N260R | G705R | L553K | Cas12i-174 | 1.06 |
| N295R | N260R | G705R | N556K | Cas12i-175 | 1.25 |
| N295R | N260R | G705R | N570K | Cas12i-176 | 1.05 |
| N295R | N260R | G705R | N571K | Cas12i-177 | Not detected |
| N295R | N260R | G705R | E793K | Cas12i-178 | 1.15 |
| N295R | N260R | G705R | N808K | Cas12i-179 | 1.16 |
| N295R | N260R | G705R | N812K | Cas12i-180 | 1.10 |
| N295R | N260R | G705R | N/A | Cas12i-69 | 1.00 |
| N/A | N/A | N/A | N/A | NC-PEI | 0.02 |
| N/A | N/A | N/A | N/A | NC | 0.02 |

TABLE 9
Results of the Eighth Round of Editing Efficiency Testing

| | | | | | Folds of editing |
|---|---|---|---|---|---|
| | | | | | efficiency |
| | | | | | compared to |
| mutation site | | | | mutant | Cas12i-30 |
| N295R | N260R | G705R | F644E | Cas12i-100 | Not detected |
| N295R | N260R | G705R | F644R | Cas12i-101 | Not detected |
| N295R | N260R | G705R | V61R | Cas12i-109 | 1.52 |
| N295R | N260R | G705R | P121R | Cas12i-120 | 1.21 |
| N295R | N260R | G705R | N449E | Cas12i-121 | 0.97 |
| N295R | N260R | G705R | K926R | Cas12i-124 | 0.63 |
| N295R | N260R | G705R | L484R | Cas12i-141 | Not detected |
| N295R | N260R | E601R | P605R | Cas12i-53 | 1.31 |
| N295R | N260R | G705R | V446R + | Cas12i-82 | 1.38 |
| | | | E788R + | | |
| | | | S811R | | |
| N295R | N260R | G705R | N168E | Cas12i-84 | 0.98 |
| N295R | N260R | G705R | N456E | Cas12i-91 | 1.08 |
| N295R | N260R | N/A | N/A | Cas12i-30 | 1.00 |
| N/A | N/A | N/A | N/A | NC-PEI | 0.03 |
| N/A | N/A | N/A | N/A | NC | 0.02 |

### Example 3: Editing Efficiency of Endogenous Genes by Cas12i Mutants

To verify the gene editing activity of the Cas12i mutant combined with gRNA in 293T cells, gRNA molecules were designed targeting the TTR, HBB and HBG target genes in 293T cells as follows (SEQ ID NO: 10-12), where the underlined part is the guide sequence (SEQ ID NO: 14-16) and the others are direct repeat sequences (DR, SEQ ID NO: 17).

gRNA-TTR:
(SEQ ID NO: 10)
AGAGAATGTGTGCATAGTCACAC CAGTAAGATTTGGTGTCTAT
gRNA-HBB:
(SEQ ID NO: 11 )
AGAGAATGTGTGCATAGTCACAC TATGCAGAAATATTGCTATTGCCT
gRNA-HBG:
(SEQ ID NO: 12 )
AGAGAATGTGTGCATAGTCACAC ACAAGGCAAACTTGACCAAT
TTR guide sequence:
(SEQ ID NO: 14)
CAGTAAGATTTGGTGTCTAT
HBB guide sequence:
(SEQ ID NO: 15)
TATGCAGAAATATTGCTATTGCCT
HBG guide sequence:
(SEQ ID NO: 16)
ACAAGGCAAACTTGACCAAT
Direct repeat sequence (DR):
(SEQ ID NO: 17)
AGAGAATGTGTGCATAGTCACAC

The gRNA expression vector (U6 promoter drives gRNA expression) was constructed. The gRNA-HBG expression vector sequence is shown in SEQ ID NO: 13. The gRNA-TTR and gRNA-HBB vectors were replaced with the corresponding gRNA coding sequences.

Plating: The 293T cell line was plated when the confluence reached 70-80%, and the number of cells seeded in a 24-well plate was 5*10\hat{ }5 cells/well.

Transfection: After 12-14 hours of plating, transfection was performed. 100 μl of Opti-MEM, 1.5 μl of PEI (Yisheng Bio, Polyethylenimine Linear (PEI) MW25000), 250 ng of the mutant clone plasmid from Example 2, and 250 ng of gRNA plasmid for targeting endogenous genes were added to each well of a 24-well plate. The mixture was incubated at room temperature for 20 minutes and then added to 293T cells for transfection. After transfection overnight, the medium was replaced with fresh medium and cultured for the next period.

DNA extraction, PCR amplification, and Sanger sequencing: After culturing for 72 hours, cells were washed with PBS, and then 100 μl of cell lysis buffer (Viagen, DirectPCR® Lysis Reagent (Cell)) was added for lysis to obtain a lysate containing genomic DNA. The genomic DNA was amplified in the region near the target sequence, and the PCR product was sent to a sequencing company for Sanger sequencing.

Sequencing data analysis: Sequencing peak diagrams and gRNA-guided sequence information were submitted to the TIDE analysis website (http://shinyapps.datacurators.nl/tide/) to obtain the editing efficiency of the mutant protein on the target nucleic acid, as shown in Tables 10-13. Among them, Cas12i-30 showed editing efficiencies higher than 10% for both TTR and HBB genes, Cas12i-69 showed editing efficiencies higher than 15% for both TTR and HBB genes, and Cas12i-69 showed an editing efficiency higher than 3% for the HBG gene.

TABLE 10
Endogenous gene editing in the second round of mutants

| | Folds of the |
|---|---|
| | editing efficiency |
| | targeting TTR gene |
| mutant | compared to wild type |
| Wild-type Cas12i control | 1.00 |
| Cas12i-30 | 1.89 |
| Cas12i-34 | 1.33 |
| Cas12i-54 | 1.47 |

TABLE 11
Endogenous gene editing in the third round of mutants

| | Folds of the editing | Folds of the editing |
|---|---|---|
| | efficiency targeting | efficiency targeting |
| | TTR gene compared to | HBB gene compared |
| mutant | Cas12i-30 | to Cas12i-30 |
| Cas12i-69 | Not detected | 1.58 |
| Cas12i-73 | 1.13 | 0.94 |
| Cas12i-30 | 1.00 | 1.00 |

TABLE 12
Endogenous gene editing in the fourth and fifth rounds of mutants.

| | Folds of the editing |
|---|---|
| | efficiency targeting TTR |
| | gene compared to |
| mutant | Cas12i-69 |
| Cas12i-69 | 1.00 |
| Cas12i-83 | 0.88 |
| Cas12i-92 | 1.24 |
| Cas12i-104 | 1.25 |
| Cas12i-113 | 1.30 |
| Cas12i-135 | 1.73 |
| Cas12i-136 | 1.23 |
| Cas12i-137 | 1.43 |
| Cas12i-96 | 0.91 |
| Cas12i-99 | 1.19 |

Among them, the Cas12i-92 mutant has N295R, N260R, G705R and P605E mutations compared to SEQ ID NO: 1.

TABLE 13
Endogenous gene editing in the fourth and fifth rounds of mutants.

| | Folds of the | Folds of the | Folds of the |
|---|---|---|---|
| | editing | editing | editing |
| | efficiency | efficiency | efficiency |
| | targeting | targeting | targeting |
| | TTR gene | HBB gene | HBG gene |
| | compared | compared | compared |
| mutant | to wild type | to Cas12i-69 | to wild type |
| Cas12i-96 | Not detected | 0.79 | 1.30 |
| Cas12i-99 | Not detected | 0.90 | 7.00 |
| Cas12i-111 | 3.91 | 1.01 | 1.53 |
| Cas12i-133 | 4.95 | 0.78 | 1.57 |
| Cas12i-69 | 4.68 | 1.00 | 1.27 |
| Wild-type | 1.00 | | 1.00 |
| Cas12i control | | | |

### Example 4: Cas12i Mutant Design and Vector Construction, and Report System Editing Efficiency Testing

The mutant expression vector plasmid was constructed using the same method as in Example 2, and the editing efficiency of the mutant in the report system was tested. The results are shown in Table 14.

TABLE 14
Editing Efficiency Test Results

| | | | | | Folds of |
|---|---|---|---|---|---|
| | | | | | the editing |
| | | | | | efficiency |
| | | | | | compared to |
| mutation site | | | | mutant | Cas12i-69 |
| N295R | N260R | G705R | N/A | Cas12i-69 | 1.0 |
| N295R | N260R | G705R | D166W | Cas12i-181 | 1.2 |
| N295R | N260R | G705R | D166F | Cas12i-182 | 1.1 |
| N295R | N260R | G705R | V167W | Cas12i-183 | 0.7 |
| N295R | N260R | G705R | V167F | Cas12i-184 | 0.8 |
| N295R | N260R | G705R | V167R | Cas12i-185 | 0.8 |
| N295R | N260R | G705R | N168W | Cas12i-186 | 0.6 |
| N295R | N260R | G705R | N168F | Cas12i-187 | 0.5 |
| N295R | N260R | G705R | G169W | Cas12i-188 | 0.5 |
| N295R | N260R | G705R | G169F | Cas12i-189 | 0.6 |
| N295R | N260R | G705R | G169R | Cas12i-190 | 0.2 |
| N295R | N260R | G705R | W170F | Cas12i-191 | 0.2 |
| N295R | N260R | G705R | S174W | Cas12i-192 | 0.1 |
| N295R | N260R | G705R | S174F | Cas12i-193 | 0.1 |
| N295R | N260R | G705R | E179R | Cas12i-194 | 0.1 |
| N295R | N260R | G705R | K181R | Cas12i-195 | 1.0 |
| N295R | N260R | G705R | K182R | Cas12i-196 | 0.3 |
| N295R | N260R | G705R | E183R | Cas12i-197 | 0.1 |
| N295R | N260R | G705R | E184R | Cas12i-198 | 0.8 |
| N295R | N260R | G705R | Q294W | Cas12i-199 | 0.2 |
| N295R | N260R | G705R | Q294F | Cas12i-200 | Not detected |
| N295R | N260R | G705R | E328R | Cas12i-201 | Not detected |
| N295R | N260R | G705R | K370R | Cas12i-202 | 1.1 |
| N295R | N260R | G705R | N372R | Cas12i-203 | 0.7 |
| N295R | N260R | G705R | E376R | Cas12i-204 | 1.1 |
| N295R | N260R | G705R | E397R | Cas12i-205 | 0.8 |
| N295R | N260R | G705R | E462R | Cas12i-206 | 1.0 |
| N295R | N260R | G705R | V463R | Cas12i-207 | 0.2 |
| N295R | N260R | G705R | N621D | Cas12i-208 | 0.0 |
| N295R | N260R | G705R | D851R | Cas12i-209 | 1.1 |
| N295R | N260R | G705R | S853R | Cas12i-210 | 1.0 |
| N295R | N260R | G705R | A934R | Cas12i-211 | 0.9 |
| N295R | N260R | G705R | W938R | Cas12i-212 | 0.2 |
| N295R | N260R | G705R | N941R | Cas12i-213 | 0.9 |
| N295R | N260R | G705R | K942R | Cas12i-214 | 1.0 |
| N295R | N260R | G705R | K943R | Cas12i-215 | 0.9 |
| N295R | N260R | G705R | N945R | Cas12i-216 | 1.0 |
| N295R | N260R | G705R | N197K | Cas12i-217 | 1.0 |
| N295R | N260R | G705R | E788R | Cas12i-218 | 1.0 |
| N295R | N260R | G705R | E788R&N197K | Cas12i-219 | 0.8 |
| N295R | N260R | G705R | K228R | Cas12i-221 | 0.6 |
| N295R | N260R | G705R | K231R | Cas12i-222 | 1.0 |
| N295R | N260R | G705R | L329R | Cas12i-224 | 0.9 |
| N295R | N260R | G705R | K353R | Cas12i-225 | 0.9 |
| N295R | N260R | G705R | P362R | Cas12i-226 | 0.0 |
| N295R | N260R | G705R | G366R | Cas12i-227 | 0.7 |
| N295R | N260R | G705R | N368R | Cas12i-228-1 | Not detected |
| N295R | N260R | G705R | N369R | Cas12i-229 | 0.9 |
| N295R | N260R | G705R | Y371R | Cas12i-231 | 0.9 |
| N295R | N260R | G705R | A392R | Cas12i-233 | 1.0 |
| N295R | N260R | G705R | K395R | Cas12i-234 | 0.7 |
| N295R | N260R | G705R | D396R | Cas12i-235 | 0.7 |
| N295R | N260R | G705R | E399R | Cas12i-236 | 0.7 |
| N295R | N260R | G705R | E400R | Cas12i-237 | 1.0 |
| N295R | N260R | G705R | K401R | Cas12i-238 | 1.1 |
| N295R | N260R | G705R | G402R | Cas12i-239 | Not detected |
| N295R | N260R | G705R | I403R | Cas12i-240 | 1.0 |
| N295R | N260R | G705R | H405R | Cas12i-241 | 0.2 |
| N295R | N260R | G705R | K408R | Cas12i-242 | 0.3 |
| N295R | N260R | G705R | E434R | Cas12i-243 | 1.0 |
| N295R | N260R | G705R | S433R | Cas12i-244 | Not detected |
| N295R | N260R | G705R | K441R | Cas12i-245 | Not detected |
| N295R | N260R | G705R | C448A | Cas12i-246 | 0.4 |
| N295R | N260R | G705R | G455R | Cas12i-248 | Not detected |
| N295R | N260R | G705R | K502R | Cas12i-249 | Not detected |
| N295R | N260R | G705R | T505R & V842I | Cas12i-250 | 0.9 |
| N295R | N260R | G705R | K580R | Cas12i-251 | 1.0 |
| N295R | N260R | G705R | T623R | Cas12i-253 | Not detected |
| N295R | N260R | G705R | S775R | Cas12i-255 | Not detected |
| N295R | N260R | G705R | T850R | Cas12i-257 | Not detected |
| N295R | N260R | G705R | K856R | Cas12i-258 | Not detected |
| N295R | N260R | G705R | K926R | Cas12i-259 | Not detected |
| N295R | N260R | G705R | Q929R | Cas12i-260 | Not detected |
| N295R | N260R | G705R | N930R | Cas12i-261 | Not detected |
| N295R | N260R | G705R | S940R | Cas12i-262 | Not detected |
| N295R | N260R | G705R | S944R | Cas12i-263 | Not detected |
| N295R | N260R | G705R | E326R | Cas12i-223 | 0.9 |
| N295R | N260R | G705R | K774R | Cas12i-247 | 1.0 |
| N295R | N260R | G705R | S779R | Cas12i-256 | 0.8 |
| N295R | N260R | G705R | H511N | Cas12i-264-1 | 0.9 |
| N295R | N260R | G705R | H511N & N523H | Cas12i-264-2 | 0.9 |
| N295R | N260R | G705R | P524H | Cas12i-265-1 | 1.0 |
| N295R | N260R | G705R | N523H & P524H | Cas12i-265-2 | 1.0 |
| N295R | N260R | G705R | P1032H | Cas12i-266 | 0.6 |
| N295R | N260R | G705R | P579H | Cas12i-267 | 0.7 |
| N295R | N260R | G705R | P984H | Cas12i-268 | 0.1 |
| N295R | N260R | G705R | L767M | Cas12i-269 | 0.9 |
| N295R | N260R | G705R | H995N | Cas12i-270 | 0.6 |
| N295R | N260R | G705R | P557H | Cas12i-271 | 1.0 |
| N295R | N260R | G705R | G232D | Cas12i-272 | 0.5 |
| N295R | N260R | G705R | L662M | Cas12i-273 | 1.0 |

Note:
The symbol ‘&’ indicates that there are two mutations before and after the symbol (a total of two mutations).

### Example 5: Screening of C12-102 Protein

### 1. CRISPR and Gene Annotation

Software was used to predict proteins expressed in microbial genomes from the NCBI Gebank database, and then software was used to predict CRISPR arrays on the genomes.

### 2. Preliminary Screening of Proteins

Clustering is used to remove redundant proteins, while filtering out proteins with an amino acid sequence length of less than 800 amino acids or greater than 1400 amino acids.

### 3. Obtaining CRISPR-Related Proteins

Protein sequences within 10 kb upstream and downstream of the CRISPR array were compared with known Cas12 sequences, filtering out proteins with an evalue greater than 1*e-2. Then, the sequences were compared with the NCBI NR database and the EBI patent database, filtering out proteins with high similarity. Candidate proteins were then selected. Experimental verification yielded the C12-102 protein, whose amino acid sequence is shown in SEQ ID NO: 18. The C12-102 protein is also named as the CasRfg.8 protein.

(SEQ ID NO: 18)
MEAGKVGKKGKTNKKFIIRPYLTELNLREDGRLAFQKTFDYMDEQQAALFILGGSVMSHLDE
SIIRRLGLHKGSKKDLPQRLRVSLHLAIARFRLVSVNYHLDAKAISRMSPTARLAHKQLAEAHRASLIK
SPISVWRNRHGVPDEAVHAYLDGNYDPETYAWQDTAMLAKKLCGILKLSPEDFKEASEAMMRNVN
FLGCSGSTGSGSSVSNLFGQNEKEDSRNQARIESKTAKVIGKLLESRKPIPMERAVSLVCKSLGHPDAE
AAGEDHGGQTDKSTFRQFMRGEYGGSLKELAKKLQKDAHKHRNKSIIPHRETIGAFIKQCASGEFYN
KATSESWKDFNAMMNGKYKHNIIFVTEKIALGNAMRKLESNEEAVKASKQLEKLIDKYEMDGNKRF
VPKVASVNGLEAYRDVIQDNPQEDDESVKDWLKRLWITFSEGNDRRLRTPFRWLVESLARLETPESAI
KDGCRLMSIRHKHESQRPHPFVRVQSRFTVGDSNIAGSINKPTELKPNRDGRSPEDYWGGNPVVWMS
CRLLNGNRWQDMRIPIHNSRYINEVYYTRMGPDGNHALPLKEHARDVKHDYEAETKISRTAARRVN
ENRMLRRGKPSKRFERVKANSTHNVVFDPKTTASFNRRQDDIYGTINHRHPMVPLAPDGVFAVGANI
IGIDLGESVPLAAGILQKCTSTDSEAVRYACGHWKVVGMGKPGQLLDRQTSAIRRKQPHTIIDPMSNM
GEPFSSPICQKFIGKCRKFVRAKGSEEDNKAFDDMVKREPSLYSFHGRWGWLLKQMMKAAKGARLD
PFREHLEWLLFRTKYGPTNRKSLNLNSMASTKNVISAIDSYMSRRGWKTVEQRQRRDGRLQAARSSL
QSNLVNRRLERIKKEESQCIRLAHLFAVTTHLVLEDNLPKONGASSRADNGRKADWRSRHLAQRLCG
IKNDSGSCAIAGVRARRIDPVMTSHMDPFVYSLDNKWAMRARYTKVSLSSMTDYHANLIRSILLEKP
GKRQTTEYYQRAVQDFARLHGLDVDEMRKVREACWYKKRIKAKTLYIPCRGGRVFLSTHRLQSDTP
HTDSSGAVLWEQDADQVAALNVALRYIDQCYRSNKKGLAKVKKPK.

### Example 6: Preparation and Purification of C12-102 Protein

### 1. Carrier Construction

The pET28a vector plasmid was digested with BamHI and XhoI, and the linearized vector was recovered by agarose gel electrophoresis. Using the prepared pXC12-102-GFPPAM plasmid (SEQ ID NO: 19) as a template, a DNA fragment containing the coding sequence of the C12-102 protein was obtained by PCR amplification using primers C12-102-pET28a-PF1 and C12-102-pET28a-PR1. This fragment was then inserted into the cloning region of the pET28a vector using homologous recombination (NEB, Gibson Assembly® Master Mix) to construct the recombinant vector C12-102-pET28a (SEQ ID NO: 20). The reaction solution was transformed into Stb13 competent cells, plated on kanamycin sulfate-resistant LB plates, incubated overnight at 37° C., and clones were picked for sequencing identification. The sequences of primers C12-102-pET28a-PF1 and C12-102-pET28a-PR1 are as follows:

Primer C12-102-pET28a-PF1 (SEQ ID NO: 21):
ACAGCAAATGGGTCGCGGATCCATGCCGGCAGCTAAGAAAAAGAAACTGG
ATGGCAGCG
Primer C12-102-pET28a-PR1 (SEQ ID NO: 22):
TCTCAGTGGTGGTGGTGGTGGTGCTCGAGTCAAGCGTAATCTGGAACATC
GTATG

Positive clones with correct sequences were selected and cultured overnight. Plasmids were extracted and transformed into the expression strain Rosetta (DE3). The plasmids were then plated on LB agar plates containing kanamycin sulfate and cultured overnight at 37° C.

### 2. Protein Expression

Pick a single clone and inoculate it into 5 ml of LB medium containing kanamycin sulfate, and incubate overnight at 37° C.

Transplant the culture medium into 500 ml of LB medium containing kanamycin sulfate at a ratio of 1:100, incubate at 37° C. with a rotation speed of 220 rpm until OD 0.6, add IPTG to a final concentration of 0.2 mM, and induce at 16° C. for 24 h.

Wash with 15 ml PBS, centrifuge to collect bacterial cells, add lysis buffer and sonicate to disrupt, centrifuge at 10000 g for 30 min to obtain supernatant containing recombinant protein, filter the supernatant through a 0.45 μm filter membrane and then load onto column for purification.

### 3. Protein Purification

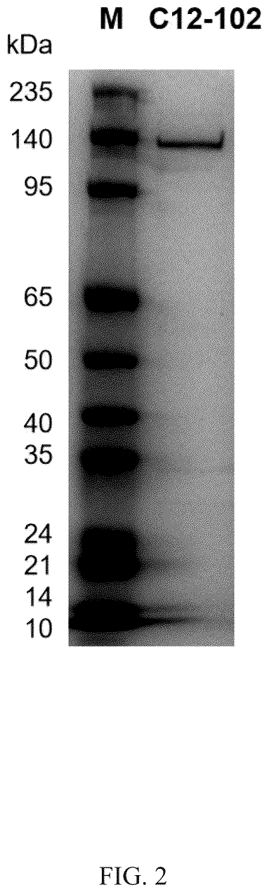

The C12-102 recombinant protein has 1213 amino acids and its structure is His tag-NLS-C12-102-SV40 NLS-nucleoplasmin NLS. Using the six N-terminal His molecules as purification tags, the protein was purified by IMAC (Ni Sepharose 6 Fast Flow, Cytiva) followed by hydrophobic chromatography (HiTrap phenly HP, Cytiva) to obtain the C12-102 recombinant protein. The purified recombinant protein was analyzed by SDS-PAGE electrophoresis, and the results are shown in FIG. 2.

### Example 7: Determination of the PAM Sequence of the C12-102 Protein

In this example, sgRNA (single guide RNA) containing a specific guide sequence and the C12-102 recombinant protein purified in Example 6 were mixed, and the substrate (containing a spacer sequence and a 7 nt random sequence) was cleaved in vitro. After incubation at 37° C., the mixture was purified, a library was constructed, and NGS sequencing and analysis were performed to determine the PAM sequence of C12-102. The specific steps are as follows:

### A. External Cutting Substrate

The designed in vitro cleavage substrate sequence
(SEQ ID NO: 23) is as follows:
ggagttcagacgtgtgctcttccgatctcagcacaaaaggaaactcaccc
taactgtaaagtaattgtgtgttttgagactataaatatgcatgcgagaa
aagccttgtttgccaccatGGAACGGCTCGGAGATCATCATTGCGNNNNN
NNgtgagcaagggcgaggagctgttcaccggggtggtgcccatcctggtc
gagctggacggcgacgtaaacggccacaagttcagcgtgtccggcagate
ggaagagcacacgtctgaactcc

In the sequence, N represents any one of A, T, C, and G.

Double-stranded DNA containing the above sequence was prepared using PCR amplification and used as an in vitro cleavage substrate.

The cleavage substrate was sent to a sequencing company for PCR-Free library construction and NGS sequencing. Complexity and abundance were analyzed for the PAM library composed of 7 nt random sequences. The results are as follows:

The four bases A, T, G, and C are basically consistent; at the same time, the PAM library composed of 7 nt random sequences contains 4\hat{ }7=16384 different combinations, all of which were detected. The complexity and abundance of the PAM library are satisfactory.

Preparation of B.sgRNA

sgRNA containing a specific guide sequence was synthesized in vitro at 37° C. using a DNA template system containing T7 RNA transcriptase, four ribonucleotide triphosphates, and a T7 promoter. The transcription product was precipitated and purified using LiCl. The sgRNA sequence is as follows:

>C12-102-sgRNA (SEQ ID NO: 24)
5′-ccucgacuagauuuagaaugcccacgauugggcaGUGAGCAAGGGCG
AGGAGCUGUUC-3′
>C12-102-sgRNA-Rev (SEQ ID NO: 25)
5′-ccucgacuagauuuagaaugcccacgaugauugggcaCGCAAUGAUG
AUCUCCGAGCCGUUCC-3′
Direct repeat sequence (SEQ ID NO: 26):
ccucgacuagauuuagaaugcccacgaugauugggca

Uppercase Bases are the Specific Guide Sequences for sgRNA:

C12-102-sgRNA guide sequence (SEQ ID NO: 27):
GUGAGCAAGGGCGAGGAGCUGUUC
C12-102-sgRNA-Rev guide sequence (SEQ ID NO: 28):
CGCAAUGAUGAUCUCCGAGCCGUUCC.

### C.NGS Library Construction and PAM Analysis

### PAM Library Cleavage and T4 DNA Polymerase Treatment

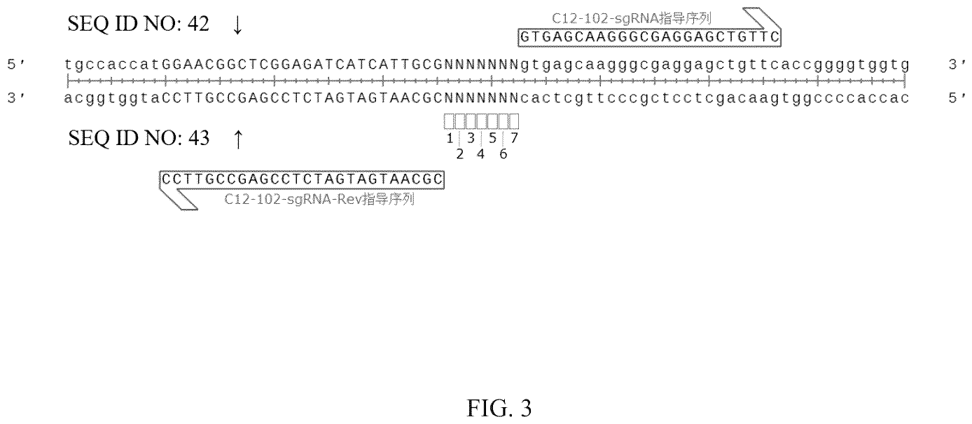

1. Prepare reaction systems containing C12-102 protein, two different sgRNAs, in vitro cleavage substrates, and buffer solutions. Incubate at 37° C. for 3 hours, followed by incubation at 75° C. for 15 minutes. See Table 15 and FIG. 3.

TABLE 15
Reaction system for in vitro cleavage reaction

| Components | Amount added (μl) |
|---|---|
| 10 × Cut Buffer(200 mM HEPES, 1M NaCl, | 5 |
| 50 mM MgCl₂, 1 mM EDTA) | |
| Cutting substrate (59.5 ng/μl) | 36.5 |
| C12-102-sgRNA or C12-102-sgRNA-Rev | 2.8 μg |
| C12-102 protein (10 mg/ml) | 0.5 |

### 2. T4 DNA Polymerase Treatment to Fill in the Cleavage Products

Add T4 DNA Polymerase (Thermo Scientific) to the cleaved product. The specific reaction system is shown in Table 16. After addition, react at 37° C. for 20 min and then at 85° C. for 10 min.

TABLE 16
Reaction system for C12-102 cutting product compensation

| Components | Amount added (μl) |
|---|---|
| C12-102 cutting products | 50 |
| 5xT4 DNA Polymerase Buffer | 13 |
| T4 DNA Polymerase | 1 |
| dNTP(10 mM) | 0.65 |
| ddH₂O | 0.35 |

3.3′ End with Added a and Added Biotin-Tagged Adapter

a. Add 78 μl of SPRISelect Beads (Beckman COULTER) to the T4 DNA Polymerase reaction product, mix well, incubate at room temperature for 5 min, transfer the product to a magnetic rack for adsorption for 5 min, transfer the supernatant to a new 1.5 ml tube; add another 39 μl of SPRISelect Beads (Beckman COULTER), mix well, incubate at room temperature for 5 min, transfer the product to a magnetic rack for adsorption for 5 min, discard the supernatant, wash twice with 85% ethanol, air dry at room temperature for 10 min, and elute with 50 μl of _{ddH2O}.

b. Using the SynplSeq DNA Library Prep Kit for Illumina, perform 3′ addition of A on the product in a according to the system in Table 17:37° C. for 10 min, 65° C. for 20 min, and 4° C. for ∞.

TABLE 17
C12-102 cleavage product 3′ plus A

| Components | Amount added (μl) |
|---|---|
| C12-102 different sgRNA cleavage products | 49 |
| Enhancer | 1.5 |
| End Prep Mix | 6 |
| End Prep Buffer 1 | 3 |
| ddH₂O | 0.5 |
| Total | 60 |

c. Adapter 1 was obtained by annealing the upstream primer

5′Biosg/gttgacatgctggattgagacttcctacactctttccctacacgacgctcttccgatc*t (SEQ ID NO: 29), where * indicates that the t base is modified with phosphorothioate (phosphorothioate) and the downstream primer gatcggaagagcgtcgtgtagggaaagagtgtaggaagtctcaatccagcatgtcaac (SEQ ID NO: 30). Adapter 1 was added to the system according to Table 18, and the reaction was carried out at 20° C. for 30 min, followed by overnight reaction at 16° C. The reaction product was purified using SPRISelect Beads.

TABLE 18
Reaction System with Adapter 1

| Components | Amount added (μl) |
|---|---|
| The reaction products in b | 60 |
| Adapter 1 | 2 |
| DNA Ligase | 1.2 |
| 3x Ligation Buffer 1 | 33 |
| ddH₂O | 3.8 |
| Total | 100 |

d. The reaction product was purified using streptavidin-labeled magnetic beads Dynabeads ® M-280 Streptavidin (Invitrogen).

e. Recover PCR

Design the primers in Table 19, and perform Recover PCR reactions using Q5® Hot Start High-Fidelty 2× Master Mix (NEB) according to the system in Table 20 and the reaction procedure in Table 21.

TABLE 19
Recover PCR Primers

| Primer ID | sequence |
|---|---|
| Recovery PCR Forward | ggagttcagacgtgtgctc(SEQ ID NO: 31) |
| Recovery PCR Reverse | gttgacatgctggattgagacttc(SEQ ID NO: 32) |

TABLE 20
Recover PCR reaction system

| Components | Amount added (μl) |
|---|---|
| streptavidin-labeled magnetic bead | 22.5 |
| purification products | |
| Recovery PCR Forward(10 μM) | 2.5 |
| Recovery PCR Reverse(10 μM) | 2.5 |
| Q5 Hot-Start 2x Master Mix | 22.5 |
| ddH₂O | Up to 50 |

TABLE 21
Recover PCR reaction procedure

| | | |
|---|---|---|
| reaction temperature | Duration | Cycle number |

| | | | | |
|---|---|---|---|---|
| 98° | C. | 2 | min | 1 |
| 98° | C. | 10 | sec | 12 |
| 61° | C. | 30 | sec | |
| 72° | C. | 2 | min | |
| 72° | C. | 2 | min | 1 |

| | | |
|---|---|---|
| 4° | C. | ∞ |

f. Transfer the Recovery PCR product to a magnetic rack and let it adsorb for 5 min. Transfer the supernatant to a new 1.5 ml centrifuge tube. Take 3 μl of Recovery PCR product and dilute it with 148.5 μl of ddH2O.

g. Index PCR

Use the primers in Table 22 and perform Index PCR according to the system in Table 23 and the reaction procedure in Table 24.

TABLE 22
Index PCR Primers

| Primers | sequence |
|---|---|
| IF501 | aatgatacggcgaccaccgagatctacactatagcctaca |
| | ctctttccctacacgacg(SEQ ID NO: 33) |
| IR701 | caagcagaagacggcatacgagatcgagtaatgtgactgg |
| | agttcagacgtgtgctc(SEQ ID NO: 34) |

TABLE 23
Index PCR Reaction System

| Components | Amount added (μl) |
|---|---|
| Recovery PCR dilution products | 12 |
| IF501(10 μM) | 4 |
| IR701(10 μM) | 4 |
| Q5 Hot-Start 2x Master Mix | 20 |
| Total | 40 |

TABLE 24
Index PCR Reaction Procedure

| | | |
|---|---|---|
| reaction temperature | Duration | Cycle number |

| | | | |
|---|---|---|---|
| 98° C. | 2 | min | 1 |
| 98° C. | 10 | sec | 12 |
| 60° C. | 30 | sec | |
| 72° C. | 2 | min | |
| 72° C. | 2 | min | 1 |

| | |
|---|---|
| 4° C. | ∞ |

h. Index PCR products were purified by adding 0.7x SPRISelect Beads, followed by elution with 38 μl ddH₂O. Concentrations were determined using a Qubit analyzer, with the C12-102-sgRNA library concentration at 35.4 ng/ul and the C12-102-sgRNA-Rev library concentration at 35.6 ng/μl, meeting the requirements for NGS sequencing.

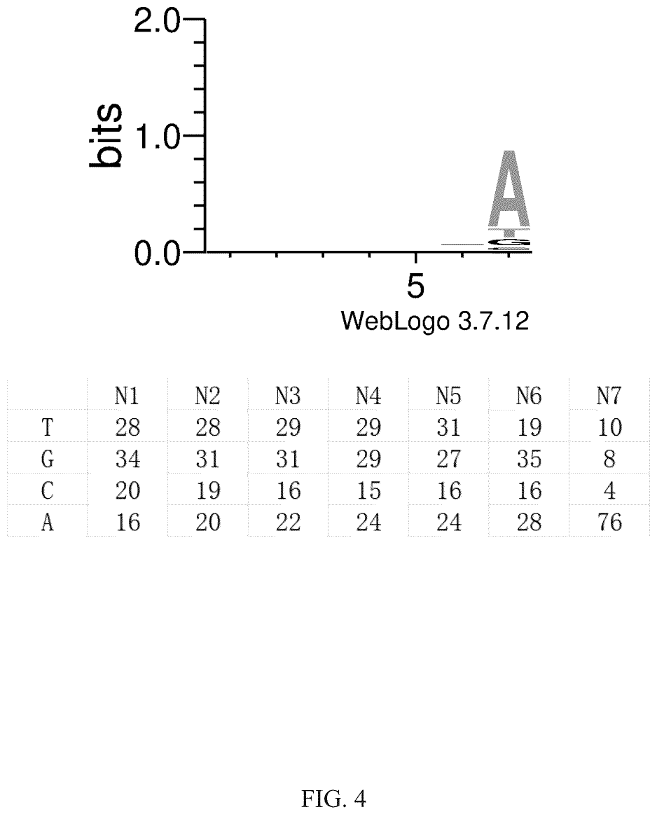

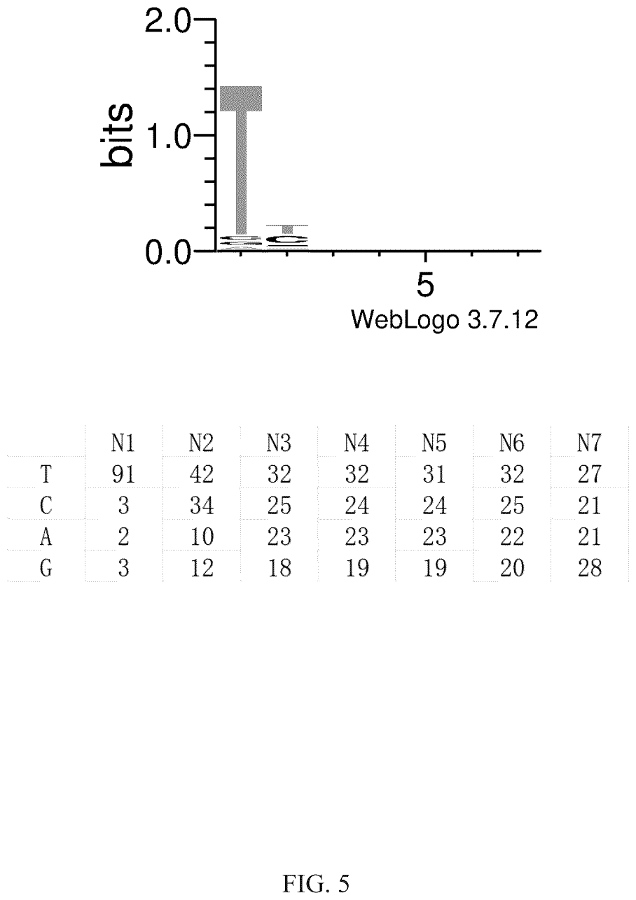

i. Analysis of NGS results: The NGS results were analyzed using WebLogo software by NGS sequencing and the method described in the reference (A compact Cas9 ortholog from Staphylococcus Auricularis (SauriCas9) expands the DNA targeting scope. PLOS biology, 2020, 18 (3), e3000686.) to obtain the captured 7 nt random sequences shown in FIGS. 4 and 5.

The PAM sequence identified using the C12-102-sgRNA system is A (FIG. 4). Since the target strand of the C12-102-sgRNA-Rev system is positive and the non-target strand is negative during targeted editing, the PAM sequence identified using the C12-102-sgRNA-Rev system is also A (FIG. 5), which is consistent with the PAM sequence identified by the C12-102-sgRNA system.

### Example 8: Testing the In Vitro Cleavage Activity of C12-102 Protein

In this example, the sgRNA from example 7 and the C12-102 recombinant protein from Example 6 are mixed and the target DNA (dsDNA or ssDNA) is cleaved in vitro. The specific steps are as follows:

a. In Vitro Cleavage of dsDNA

After binding to sgRNA, CRISPR-Cas protein can specifically cleave dsDNA containing a specific PAM. The cleavage product can be visualized by gel electrophoresis to show the cleavage effect of Cas protein.

The target DNA (dsDNA) was prepared, and its
sequence is as follows (SEQ ID NO: 35):
GGGATTGGGGGGTACAGTGCAGGGGAAAGAATAGTAGACATAATAGCAAC
AGACATACAAACTAAAGAATTACAAAAACAAATTACAAAATTCAAAATTT
TATCGATACTAGTATTATGCCCAGTACATGACCTTATGGGACTTTCCTAC
TTGGCAGTACATCTACGTATTAGTCATCGCTATTACCATGGTGATGCGGT
TTTGGCAGTACATCAATGGGCGTGGATAGCGGTTTGACTCACGGGGATTT
CCAAGTCTCCACCCCATTGACGTCAATGGGAGTTTGTTTTGGCACCAAAA
TCAACGGGACTTTCCAAAATGTCGTAACAACTCCGCCCCATTGACGCAAA
TGGGCGGTAGGCGTGTACGGTGGGAGGTTTATATAAGCAGAGCTCGTTTA
GTGAACCGTCAGATCGCCTGGAGACGCCATCCACGCTGTTTTGACCTCCA
TAGAAGATTctagaGCGAGAAAAGCCTTGTTTGCCACCATGGAACGGCTC
GGAGATCATCATTGCGTAAAGTGAGCAAGGGCGAGGAGCTGTTcaccggg
gtggtgcccatcctggtcgagctggacggcgacgtaaacggccacaagtt
cagcgtgtccggcgaggggagggcgatgccacctacggcaagctgaccct
gaagttcatctgcaccaccggcaagctgcccgtgccctggcccaccctcg
tgaccaccctgacctacggcgtgcagtgcttcagccgctaccccgaccac
atgaagcagcacgacttcttcaagtccgccatgcccgaaggctacgtcca
ggagcgcaccatcttcttcaaggacgacggcaactacaagacccgcgccg
aggtgaagttcgagggcgacaccctggtgaaccgcatcgagctgaagggc
atcgacttcaaggaggacggcaacatcctggggcacaagctggagtacaa
ctacaacagccacaacgtctatatcatggccgacaagcagaagaacggca
tcaaggtgaacttc

The underlined part is the corresponding sequence of the sgRNA guide sequence.

Two different sets of Cut Buffers were selected for the cutting reaction, as follows:

10×Cut Buffer 1:200 mM HEPES, 1M NaCl, 50 mM MgCl₂, 1 mM EDTA

10×Cut Buffer 2: 200 mM Tris-HCl (pH7.5), 500 mM KCl, 50 mM MgCl₂, 5 mM DTT, 10% glycerol, 1 mM ATP.

### Prepare the Reaction System According to Table 25:

TABLE 25
C12-102 in vitro cleavage reaction system

| Components | Amount added (μL) |
|---|---|
| 10 × Cut Buffer | 5 |
| In vitro cleavage of target DNA fragments | 500 of |
| C12-102 or C12-102-Rev sgRNA | Approximately 2.8 μg |
| C12-102 recombinant protein (10 mg/ml) | 0.5 |
| ddH₂O | Up to 50 |

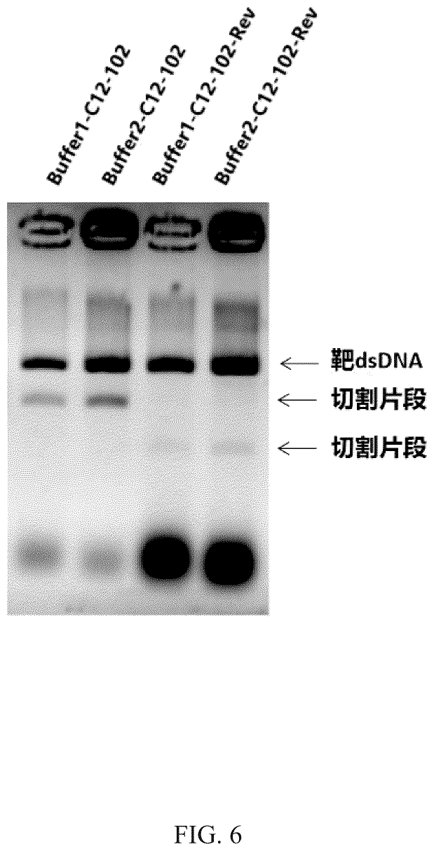

The reaction was carried out at 37° C. for 2 hours and at 75° C. for 10 minutes. 20 μl of the cleavage product was then subjected to gel electrophoresis. The electrophoresis results are shown in FIG. 6.

b. In Vitro Cleavage of ssDNA

Based on the sgRNA (C12-102 and C12-102-Rev) guide sequences in Example 6, the inventors designed ssDNA containing a 5′ FAM fluorescent group and a 3′ quencher group (3′BHQ1/3′Super Quencher 1) as a cleavage substrate. Once the C12-102 protein specifically cleaves the ssDNA (called cis cleavage), a fluorescent signal can be detected by qPCR, and the cis cleavage activity of the C12-102 protein can be determined based on the change in the fluorescent signal.

The in vitro cleavage sequence of the target DNA (ssDNA) by the C12-102 protein is as follows:

C12Template01 (SEQ ID NO: 36):
5′FAM-AACATAAtCgaacagctcctcgcccttgctcacTAGACAAtc-
3′BHQ1
C12Template02 (SEQ ID NO: 37):
5′FAM-AACATAAtCgaacagctcctcgcccttgctcacTAGACAAtc-
3′Super Quencher 1
C12Template-Rev01 (SEQ ID NO: 38):
5′FAM-tgcTaccATGGAACGGCTCGGAGATCATCATTGCGtaaAGgAt
c-3′BHQ1
C12Template-Rev02 (SEQ ID NO: 39):
5′FAM-tgcTaccATGGAACGGCTCGGAGATCATCATTGCGtaaAGgAt
c-3′Super Quencher 1

The underlined part represents the target sequence of C12-102 or C12-102-Rev sgRNA.

### Prepare the Reaction System According to Table 26:

TABLE 26
Reaction system for C12-102 in vitro ssDNA cleavage

| Components | Amount added (μL) |
|---|---|
| 10 × Cut Buffer | 2 |
| SSDNA(100 pmol) | 1 |
| C12-102 or C12-102-Rev sgRNA in Example 7 | Approximately 1.6 μg |
| Recombinant C12-102 protein (10 mg/ml) | 0.5 |
| in Example 6 | |
| RNase Inhibitor | 1 |
| ddH₂O | Up to 20 |

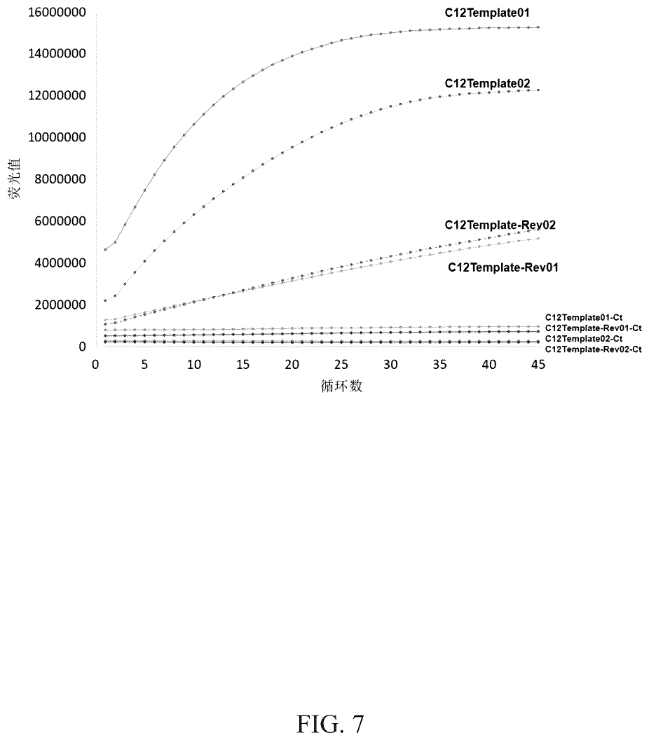

In the ssDNA cleavage experiment, all components except ssDNA were first mixed thoroughly and incubated at 25° C. for 20 min. Then, ssDNA was added, and the mixture was placed in a qPCR instrument at 37° C. for reaction. The fluorescence signal intensity was detected once per minute for each cycle. A control group without sgRNA was also set up. The fluorescence intensity and reaction time were plotted to determine the cleavage activity of the Cas protein. The results are shown in FIG. 7, where Ct represents the negative control group (containing C12-102 recombinant protein and ssDNA, but without sgRNA). The fluorescence test results show that C12-102 can specifically cleave ssDNA, and this cleavage behavior does not require the assistance of PAM (sequence A).

### Example 9: C12-102 Protein Mutant and Cas12i-Y2 Mutant

The inventors designed mutants of C12-102 and Cas12-Y2 proteins (SEQ ID NO: 40) as shown in Table 27, and tested their editing efficiency and off-target effects.

The direct repeat sequence of the sgRNA of
Cas12-Y2 (SEQ ID NO: 41):
acucgacuag auuugaaug cccacgauga uugggaca

TABLE 27
Designed mutants

| Mutant of reference protein | Mutant of reference protein |
|---|---|
| C12-102 | Cas12-Y2 |
| S211R | S211R |
| Q216R | Q216R |
| Q216F | Q216F |
| Q216W | Q216W |
| N217R | N217R |
| N217F | N217F |
| N217W | N217W |
| E218R | E218R |
| K219R | K219R |
| E220R | E220R |
| K351R | K351R |
| H352R | H352R |
| N353R | N353R |
| I355R | I355R |
| E359R | E359R |
| A362R | A362R |
| L363R | L363R |
| A366R | A366R |
| N365R | N365R |
| L370R | L370R |
| K401R | K401R |
| V402R | V402R |
| A403R | A403R |
| E439R | E439R |
| E463R | E463R |
| D468R | D468R |
| D276R | D276R |
| E287R | D287R |
| D270R | D270R |
| E265R | E265R |
| N224R | N224R |
| D413R | D413R |
| D417R | D417R |
| A410R | A410R |
| D428R | D428R |
| E424R | E424R |
| Q1005R | Q1005R |
| N991R | N991R |
| E999R | E999R |
| L998R | L998R |
| S995R | S995R |
| D762R | D762R |
| E761R | E761R |
| N763R | N763R |
| S843R | S843R |
| S836R | S836R |
| N833R | N833R |
| A829R | A829R |
| D768R | D768R |
| Y988R | Y988R |
| E24X | E24X |
| K76X | K76X |
| Q80X | Q80X |
| Q282X | Q282X |
| L254X | L254X |
| L240X | L240X |
| E241X | E241X |
| D302X | D302X |
| N441X | N441X |
| D393X | D393X |
| G394X | G394X |
| N395X | N395X |
| S481X | S481X |
| D157X | D157X |
| E159X | E159X |
| Q491X | Q491X |
| V490X | V490X |
| H485X | H485X |
| D903X | D903X |
| D953X | D953X |
| N904X | N904X |
| V955X | V955X |
| Q908X | Q908X |
| L932X | L932X |
| S939X | S939X |
| Q930X | Q930X |
| N870X | N870X |
| E851X | E851X |
| Q854X | Q854X |
| V850X | V850X |
| N873X | N873X |
| V872X | V872X |
| D839X | D839X |
| Q868X | Q868X |
| D800X | D800X |
| E804X | E804X |
| R19 mutated to K, A, Q, or E | R19 mutated to K, A, Q, or E |
| R28 mutated to K, A, Q or E | R28 mutated to K, A, Q or E |
| R32 mutated to K, A, Q or E | R32 mutated to K, A, Q or E |
| K512R | K512R |
| N527R | N527R |
| W531R | W531R |
| R553 mutated to K, A, Q or E | R553 mutated to K, A, Q or E |
| K581R | K581R |
| K589R | K589R |
| I590R | I590R |
| R605 mutated to K, A, Q or E | R605 mutated to K, A, Q or E |
| K611R | K611R |
| R612 mutated to K, A, Q or E | R612 mutated to K, A, Q or E |
| R615 mutated to K, A, Q or E | R615 mutated to K, A, Q or E |
| Y777R | Y777R |
| E877R | E877R |
| R931 mutated to K, A, Q or E | R931 mutated to K, A, Q or E |
| H271R | H271R |
| D393R | D393R |
| N395R | N395R |
| I435R | T435R |
| T436R | T436R |
| F437R | F437R |
| S438R | S438R |
| D498R | D498R |
| D639R | D639R |
| D640R | D640R |
| V850R | V850R |
| T1006R | T1006R |
| K119R | E119R |
| G205R | G205R |
| G205Y | G205Y |
| S206R | S206R |
| S206Y | S206Y |
| G207R | G207R |
| G207Y | G207Y |
| Insert NWNN between positions | Insert NWNN between positions |
| 206 and 207 (aa) | 206 and 207 (aa) |
| Insert NQYN between positions | Insert NQYN between positions |
| 206 and 207 (aa) | 206 and 207 (aa) |
| S208R | S208R |
| S208Y | S208Y |
| D221R | D221R |
| S222R | S222R |
| K244R | |
| E287R | D287R |
| K369R | K369R |
| E371R | E371R |
| S372R | S372R |
| N373R | N373R |
| E374R | E374R |
| E375R | K375R |
| N406R | N406R |
| G407R | G407R |
| E409R | E409R |
| Q416R | Q416R |
| N418R | N418R |
| E463R | E463R |
| S464R | S464R |
| K467R | K467R |
| T495R | T495R |
| V496R | V496R |
| G497R | G497R |
| D498R | D498R |
| S499R | S499R |
| N500R | N500R |
| I501R | I501R |
| Q638R | Q638R |
| D639R | D639R |
| D640R | D640R |
| I641R | I641R |
| I719R | N719R |
| G1002R | G1002R |
| K1003R | K1003R |
| C1035R | |

While specific examples of the present disclosure have been described above, those skilled in the art should understand that these are merely illustrative examples, and various changes or modifications can be made to these examples without departing from the principles and essence of the present disclosure. Therefore, the scope of protection of the present disclosure is defined by the appended claims.

What is claimed is:

1. A Cas12 protein, wherein the Cas12 protein comprises an amino acid sequence having at least 90% sequence identity to SEQ ID NO: 1, and has an amino acid difference compared with SEQ ID NO: 1 at one, two, or more positions selected from the following:
N260, N295, T235, D233, S259, Q256, M253, F680, T550, Y668, S246, N229, D678, E875, D166, N325, N168, N884, N369, N879, P605, K872, N456, E601, Q11, N443, D876, E788, G705, V446, S811, E321, E815, A869, V804, N317, N807, H702, V359, K787, P355, K703, V790, L778, D782, N409, D704, D356, T354, M863, L332, Q971, A857, Q262, C567, S849, D590, A933, F962, N930, A794, V58, L475, V61, L526, V469, Q929, L438, N449, L553, K926, T850, 1249, T313, Q450, Y881, R606, Q632, G845, N846, R860, F644, E271, E255, E328, E418, N193, N194, N556, N416, N197, N808, E504, E793, Q186, N812, N570, P121, E658, L662, 1549, D551, S664, E681, Q294, E225, N663, Y241, W170, S174, M789, S306, C448, I407, K310, C866, 11031, M618, N571 or L484.

2. The Cas12 protein of claim 1, wherein the amino acid difference is a substitution of the residue at the position with any other residue, or a deletion of the residue at the position.

3. The Cas12 protein of claim 1, wherein the amino acid difference is a substitution of the residue at the position with any other residue.

4. The Cas12 protein of claim 1, wherein the Cas12 protein comprises a sequence having at least 95% sequence identity to SEQ ID NO: 1.

5. The Cas12 protein of claim 1, wherein the Cas12 protein forms a CRISPR complex with a guide polynucleotide, and the guide polynucleotide guides the CRISPR complex to bind to a target nucleic acid.

6. The Cas12 protein of claim 1, wherein a gene-editing efficiency of the Cas12 protein is increased by at least 10% compared with that of the Cas12 protein having the sequence of SEQ ID NO: 1.

7. The Cas12 protein of claim 1, wherein the Cas12 protein has an amino acid difference at one, two, or more positions selected from the following:
N260, N295, or G705.

8. The Cas12 protein of claim 1, wherein the Cas12 protein has amino acid differences at positions N260 and N295, and further has an amino acid differences at position: E875, D166, N325, N884, N369, N879, P605, K872, N456, D678, E601, Q11, N168, D233, N443, Q450, T313, E788, V446, S811, E321, E815, A869, V804, N317, N807, H702, V359, K787, P355, K703, V790, L778, D782, N409, D704, T235, D356, D876, T354, M863, M789, S306, C448, 1407, K310, or G705.

9. The Cas12 protein of claim 1, wherein the Cas12 protein has amino acid differences at positions N260, N295, and G705, and further has an amino acid differences at position: L332, Q971, A857, P355, Q262, C567, S849, D590, A933, F962, N930, A794, N879, K872, N325, V58, L475, V61, N884, N409, L526, V469, Q929, Q11, L438, N369, N449, L553, K926, T850, 1249, T313, T354, N443, N317, Q450, Y881, R606, A869, Q632, G845, N846, R860, F644, C866, I1031, M618, E271, E255, E328, E418, N193, N194, N556, Q256, N416, N197, N808, E504, E793, Q186, N812, N570, N571, P605, P121, N456, N168, L484, or D166.

10. The Cas12 protein of claim 1, wherein the Cas12 protein has amino acid differences at positions:
N295 and N260;
N295, N260 and E875;
N295, N260 and D166;
N295, N260 and N325;
N295, N260, D166 and N168;
N295, N260 and N884;
N295, N260 and N369;
N295, N260 and N879;
N295, N260 and P605;
N295, N260 and K872;
N295, N260 and N456;
N295, N260 and D678;
N295, N260 and E601;
N295, N260 and Q11;
N295, N260 and N168;
N295, N260 and D233;
N295, N260 and N443;
N295, N260, K872, E875, D876, N879 and N884;
N295, N260 and Q450;
N295, N260, T313, N317 and N325;
N295, N260 and T313;
N295, N260 and E788;
N295, N260 and G705;
N295, N260 and V446;
N295, N260 and S811;
N295, N260 and E321;
N295, N260 and E815;
N295, N260 and A869;
N295, N260 and V804;
N295, N260 and N317;
N295, N260 and N807;
N295, N260 and H702;
N295, N260 and V359;
N295, N260 and K787;
N295, N260 and P355;
N295, N260 and K703;
N295, N260 and V790;
N295, N260 and L778;
N295, N260 and D782;
N295, N260 and N409;
N295, N260 and D704;
N295, N260 and T235;
N295, N260 and D356;
N295, N260 and D876;
N295, N260 and T354;
N295, N260 and M863;
N295, N260 and M789;
N295, N260 and S306;
N295, N260 and C448;
N295, N260 and I407;
N295, N260 and K310;
N295, N260, G705 and L332;
N295, N260, G705 and Q971;
N295, N260, G705 and A857;
N295, N260, G705 and P355;
N295, N260, G705 and Q262;
N295, N260, G705 and C567;
N295, N260, G705 and S849;
N295, N260, G705 and D590;
N295, N260, G705 and A933;
N295, N260, G705 and F962;
N295, N260, G705 and N930;
N295, N260, G705 and A794;
N295, N260, G705 and N879;
N295, N260, G705 and K872;
N295, N260, G705 and N325;
N295, N260, G705 and V58;
N295, N260, G705 and L475;
N295, N260, G705 and V61;
N295, N260, G705 and N884;
N295, N260, G705 and N409;
N295, N260, G705 and L526;
N295, N260, G705 and V469;
N295, N260, G705 and Q929;
N295, N260, G705 and Q11;
N295, N260, G705 and L438;
N295, N260, G705 and N369;
N295, N260, G705 and N449;
N295, N260, G705 and L553;
N295, N260, G705 and K926;
N295, N260, G705 and T850;
N295, N260, G705 and I249;
N295, N260, G705 and T313;
N295, N260, G705 and T354;
N295, N260, G705 and N443;
N295, N260, G705 and N317;
N295, N260, G705 and Q450;
N295, N260, G705 and Y881;
N295, N260, G705 and R606;
N295, N260, G705 and A869;
N295, N260, G705 and Q632;
N295, N260, G705 and G845;
N295, N260, G705 and N846;
N295, N260, G705 and R860;
N295, N260, G705 and F644;
N295, N260, G705 and C866;
N295, N260, G705 and I1031;
N295, N260, G705 and M618;
N260, G705 and E788;
N260, G705, D166 and N168;
N260, G705 and V446;
N260, G705 and S811;
N295, N260, G705 and E271;
N295, N260, G705 and E255;
N295, N260, G705 and E328;
N295, N260, G705 and E418;
N295, N260, G705 and N193;
N295, N260, G705 and N194;
N295, N260, G705 and N556;
N295, N260, G705 and Q256;
N295, N260, G705 and N416;
N295, N260, G705 and N197;
N295, N260, G705 and N808;
N295, N260, G705 and E504;
N295, N260, G705 and E793;
N295, N260, G705 and Q186;
N295, N260, G705 and N812;
N295, N260, G705 and N570;
N295, N260, G705 and N571;
N295, N260, G705, V446, E788 and S811;
N295, N260, E601 and P605;
N295, N260, G705 and P121;
N295, N260, G705 and N456;
N295, N260, G705 and N168; or,
N295, N260, G705 and L484.

11. The Cas12 protein of claim 1, wherein the amino acid difference is selected from the following:
N260R, N295R, T235R, D233R, S259R, Q256R, M253R, F680R, T550R, Y668R, S246R, N229R, D678R, E875R, D166R, N325R, N168R, N884R, N369R, N879R, P605R, K872R, N456R, D678E, E601R, Q11R, N443R, D876R, E788R, G705R, V446R, S811R, E321R, E815R, A869R, V804R, N317R, N807R, H702R, V359R, K787R, P355R, K703R, V790R, L778R, D782R, N409R, D704R, D356R, T354R, M863R, L332R, Q971R, A857R, P355E, Q262E, Q971E, C567R, S849R, D590R, A933E, F962E, N930R, A794R, N879E, L332E, F962R, K872E, N325E, V58R, L475R, V61E, N884E, N409E, L526E, V469R, L526R, Q929R, Q11E, S849E, L438R, A857E, A933R, Q929E, N369E, N449R, L553R, Q262R, K926E, T850R, 1249F, T313E, I249R, T354E, I249E, N443E, N317E, T850E, Q450E, K872N, Y881H, R606K, A869G, Q632E, G845A, N846D, R860S, F644Y, E271K, E255K, E328K, E418K, N193K, N194K, N556K, Q262K, Q256K, N416K, N197K, N808K, E504K, E793K, Q186K, N812K, L553K, N570K, L475K, V61R, P121R, N456E, N168E, N449E, K926R, E658R, L662R, 1549R, D551R, S664R, E681R, Q294R, E225R, N663R, Y241R, W170R, S174R, Q450R, T313R, M789R, S306R, C448R, 1407R, K310R, C866L, I1031K, M618Y, N571K, L484R, F644E and F644R.

12. The Cas12 protein of claim 5, wherein the guide polynucleotide comprises a direct repeat sequence linked to a guide sequence, and has at least 80% sequence identity to SEQ ID NO: 17.

13. The Cas12 protein of claim 1, wherein the Cas12 protein recognizes a 5′-TTN PAM sequence, wherein N is A, T, C, or G.

14. A fusion protein or conjugate, wherein the fusion protein or conjugate comprises the Cas12 protein of claim 1, or a functional fragment thereof.

15. A CRISPR-Cas12 system comprising:
a. the Cas12 protein of claim 1, a fusion protein or conjugate comprising the Cas12 protein, or a functional fragment thereof, or a nucleic acid encoding the Cas12 protein, the fusion protein or the conjugate;
and
b. a guide polynucleotide, or a polynucleotide sequence encoding the guide polynucleotide;
wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner.

16. The CRISPR-Cas12 system of claim 15, wherein the target nucleic acid is an eukaryotic DNA.

17. A delivery system, wherein the delivery system comprises:
(1) a delivery vehicle, and
(2) the Cas12 protein of claim 1, a fusion protein or conjugate comprising the Cas12 protein or a functional fragment thereof, or a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner.

18. The delivery system of claim 17, wherein the delivery vehicle is a lipid nanoparticle.

19. A pharmaceutical composition, wherein the pharmaceutical composition comprises: the Cas12 protein of claim 1, a fusion protein or conjugate comprising the Cas12 protein or a functional fragment thereof, a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner, or the delivery system comprising a delivery vehicle, and the Cas12 protein, the a fusion protein or conjugate comprising the Cas12 protein or a functional fragment thereof, or the a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner.

20. A method for diagnosing, treating, or preventing a disease or condition associated with a target nucleic acid, wherein the method comprises administering, to a sample from a subject in need thereof or to a subject in need thereof, the Cas12 protein of claim 1, a fusion protein or conjugate comprising the Cas12 protein or a functional fragment thereof, a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner, a delivery system comprising a delivery vehicle, and the Cas12 protein, the a fusion protein or conjugate comprising the Cas12 protein or a functional fragment thereof, or the a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner, or a pharmaceutical composition comprising the Cas12 protein, a fusion protein or conjugate comprising the Cas12 protein or a functional fragment thereof, a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner, or the delivery system comprising a delivery vehicle, and the Cas12 protein, the a fusion protein or conjugate comprising the Cas12 protein or a functional fragment thereof, or the a CRISPR-Cas12 system comprising the Cas12 protein, the fusion protein or the conjugate, and a guide polynucleotide, or a polynucleotide sequence encoding the guide polynucleotide, wherein the Cas12 protein, the fusion protein or the conjugate forms a CRISPR complex with the guide polynucleotide; and the guide polynucleotide comprises a guide sequence that is engineered to guide the CRISPR complex to bind to a target nucleic acid in a sequence-specific manner.
